SEQUENCE LISTING (15 sequences)

SEQ ID NO: 1 - protein, 123 aa, Homo sapiens
  BINDING 31..35: note = CDR1
  BINDING 50..66: note = CDR2
  BINDING 99..112: note = CDR3
QVQLVQSGAEVKKPGASVTLSCKASGYTFTAYYIHWVRQAREQGLEWMGVINPSTGTTFYAQNFPDRVSVTRDTSTSTVFMELHNLKSEDTAVYYCARAISEHGSGSYSPYYWGQGTLVTVSS

SEQ ID NO: 2 - protein, 123 aa, Homo sapiens
  BINDING 31..35: note = CDR1
  BINDING 50..66: note = CDR2
  BINDING 99..112: note = CDR3
EVQLVESGAEVKKPGASVTLSCKASGYTFTAYYIHWVRQAREQGLEWMGVINPSTGTTFYAQNFPDRVSVTRDTSTSTVFMELHNLKSEDTAVYYCARAISEHGSGSYSPYYWGQGTLVTVSS

SEQ ID NO: 3 - protein, 5 aa, Homo sapiens
AYYIH

SEQ ID NO: 4 - protein, 17 aa, Homo sapiens
VINPSTGTTFYAQNFPD

SEQ ID NO: 5 - protein, 14 aa, Homo sapiens
AISEHGSGSYSPYY

SEQ ID NO: 6 - protein, 107 aa, Homo sapiens
  BINDING 23..34: note = CDR1
  BINDING 49..55: note = CDR2
  BINDING 88..97: note = CDR3
SYELTQPPSVSVSLGQMAAITCSGEALPKKYGYWYQQKPGQVPVLLIYRDVERPSGVPDRFSGSSSGTMVTLTISGVQAEDEADYYCLSADSSGTWVFGGGTKLTVL

SEQ ID NO: 7 - protein, 107 aa, Homo sapiens
  BINDING 23..34: note = CDR1
  BINDING 49..55: note = CDR2
  BINDING 88..97: note = CDR3
SYVLTQPPSVSVSLGQMAAITCSGEALPKKYGYWYQQKPGQVPVLLIYRDVERPSGVPDRFSGSSSGTMVTLTISGVQAEDEADYYCLSADSSGTWVFGGGTKLTVL

SEQ ID NO: 8 - protein, 11 aa, Homo sapiens
SGEALPKKYGY

SEQ ID NO: 9 - protein, 7 aa, Homo sapiens
RDVERPS

SEQ ID NO: 10 - protein, 10 aa, Homo sapiens
LSADSSGTWV

SEQ ID NO: 11 - protein, 11 aa, synthetic construct
GGPKDEERHVG

SEQ ID NO: 12 - DNA, 369 bp, Homo sapiens
  source 1..369: mol_type = other DNA
gaggtgcagctggtggagtctggggctgaggtgaagaagcctggggcctcagtgacactgtcctgcaaggcatctggatacaccttcaccgcctactatatacactgggtgcgacaggcccgagaacaagggcttgagtggatgggcgtaatcaaccctagtactggaaccacattttacgcacagaacttcccggacagagtctccgtgaccagggacacgtccacgagtacagtcttcatggagctgcacaacctgaaatctgaggacacggccgtatattactgtgcgagagcaatcagtgagcatggttcagggagttattcaccttattactggggccagggcaccctggtcaccgtctcctcg

SEQ ID NO: 13 - DNA, 321 bp, Homo sapiens
  source 1..321: mol_type = other DNA
tcctatgtgctgactcagccaccctcggtgtcagtgtccctaggacagatggccgcgatcacctgctctggagaggcattgccaaaaaagtatggttattggtaccagcagaagccaggccaggtccctgttctgctaatttatagagacgtcgagaggccctcaggggtccctgaccgattctctggctccagctcagggacaatggtcacattgaccatcagtggagtccaggcagaggacgaggctgactattactgtctctcagcagacagcagtggtacttgggtgttcggcggagggaccaagctgaccgtccta

SEQ ID NO: 14 - protein, 453 aa, Homo sapiens
  REGION 124..453: note = constant region
QVQLVQSGAEVKKPGASVTLSCKASGYTFTAYYIHWVRQAREQGLEWMGVINPSTGTTFYAQNFPDRVSVTRDTSTSTVFMELHNLKSEDTAVYYCARAISEHGSGSYSPYYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 213 aa, Homo sapiens
  REGION 108..213: note = constant region
SYELTQPPSVSVSLGQMAAITCSGEALPKKYGYWYQQKPGQVPVLLIYRDVERPSGVPDRFSGSSSGTMVTLTISGVQAEDEADYYCLSADSSGTWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2026/0306683 A1
(43) Pub. Date: Oct. 8, 2026

(54) IN VIVO IMAGING OF AN ALS BIOMARKER AND MEANS THEREFOR

(71) Applicant: AL-S PHARMA AG, Schlieren (CH)

(72) Inventors: Brigitte GUÉRIN, Westbury (CA); Sébastien TREMBLAY, Sherbrooke (CA); Samia AIT-MOHAND, Sherbrooke (CA); Michael SALZMANN, Zürich (CH); Marcel MAIER, Zürich (CH)

(73) Assignee: AL-S PHARMA AG, Schlieren (CH)

(21) Appl. No.: 19/477,940

(22) PCT Filed: Apr. 24, 2024
(86) PCT No.: PCT/CA2024/050540

§ 371 Date: Oct. 23, 2025

(30) Foreign Application Priority Data
Apr. 24, 2023 (EP) 23169614.7
Jul. 24, 2023 (EP) 23187354.8

(51) Int. Cl. A61K 51/10; A61P 25/28

(52) U.S. Cl. CPC A61K 51/1096; A61K 51/1075; A61P 25/28

(57) ABSTRACT

Provided are labeled antibody conjugates for use in a method of diagnosing ALS or monitoring drug therapy and/or progression of ALS by in vivo detecting and tracing misfolded SOD1 by immunoimaging in a subject, compositions and kits comprising said conjugates and methods of in vivo immunoimaging of superoxide dismutase (SOD1) useful as a marker of amyotrophic lateral sclerosis (ALS) or efficacy of drug therapy of ALS in subject using these conjugates. Furthermore, SOD1-antibodies specific for misfolded and/or aggregated SOD1 are provided for use in treatment of bone and/or joint related diseases.

### CROSS-REFERENCE TO RELATED APPLICATIONS

This application is a U.S. National Stage Application under 35 U.S.C. § 371 of International Patent Application No. PCT/CA2024/050540, filed Apr. 24, 2024, which claims priority to European Patent Application No. 23187354.8, filed Jul. 24, 2023 and European Patent Application No. 23169614.7, filed Apr. 24, 2023, the disclosure of each of which is incorporated by reference herein in its entirety.

### SEQUENCE LISTING

This application contains an electronic Sequence Listing which has been submitted in XML file format via Patent Center, the entire content of which is incorporated by reference herein in its entirety. The Sequence Listing XML file submitted via Patent Center is entitled “14727-083-999_SL.xml”, was created on Oct. 5, 2025, and is 20,618 bytes in size.

### FIELD OF THE INVENTION

The invention relates to means for in vivo immunoimaging of misfolded SOD1 (mSOD1). More specifically, the present invention relates to antibodies and antibody conjugates, respectively, which are useful in the diagnosis and in monitoring treatment/progression of diseases due to or associated with misfolded and aggregated SOD1, in particular neurogenerative diseases such as amyotrophic lateral sclerosis (ALS). Furthermore, the invention relates to anti-SOD1 antibodies for use in targeting SOD1 in the cartilage, for example at bones and joints in subjects.

### BACKGROUND OF THE INVENTION

ALS is a rare neurodegenerative disease resulting from the loss of motor neurons in the motor cortex, brainstem, and spinal cord. It currently affects three of every 100,000 people in the US and approximately 5,000 people are diagnosed with ALS each year. The majority of patients are between 40 and 70 years of age (ALSA 2019). The typically rapid decline in neurologic function results from the death of motor neurons that enable movement, speech, swallowing and breathing. Effective denervation of these muscles results in wasting and atrophy with patients eventually requiring assisted ventilation before succumbing to respiratory failure, which is the most common cause of death in ALS (Tateno et al. 2014).

Of all ALS cases ~10% are inherited (FALS) and the rest arise sporadically (SALS). Mutations in the SOD1 gene, leading to the misfolding of its protein product, are the second most common genetic cause of ALS (Vu and Browser 2017). While the exact mechanism of mutant-SOD1 toxicity is still unknown, most evidence points to a pathological gain of function associated with the propensity of this protein to misfold and aggregate (Li and Cashman 2014, Bosco et al. 2010, Forsberg et al. 2011, Forsberg et al. 2010, Graffmo et al. 2013).

Preclinical disease modeling utilizing transgenic mice (Tg) has generated data supporting the hypothesis that misfolding of wild-type non mutated SOD1 protein occurs and can also generate a “toxic conformation” that results in an ALS like pathology (Ayers et al. 2016, Bidhendi et al. 2016). Detection of misfolded wild-type SOD1 within human post-mortem sporadic ALS samples has been used to support the hypothesis that misfolded forms of wild-type SOD1 can contribute to sporadic ALS pathogenesis (Bosco et al. 2010, Forsberg et al. 2011, Forsberg et al. 2010, Tokuda et al. 2019, Pare et al. 2018, Maier et al. 2018).

Preliminary data was generated using the CSF from a cohort of SALS patients, where results suggested that 60-70% of SALS patients were also positive for misfolded SOD1. Overall, these results along with IHC (immunohistochemistry) data suggest that the full penetrance of expression of misfolded SOD1 in these patients is significantly higher than detection from CSF would predict. Implications for this include the assumption that while the CSF assay under development is potentially a useful tool to support diagnosis of ALS, the assay does not yet provide a suitable prospective patient selection or diagnostic tool for the identification of pre-symptomatic ALS. Furthermore, an assay that relies on the detection of misfolded SOD1 in the CSF may not be suitable for all patients, as there may be factors that influence the appearance of misfolded SOD1 in the CSF vs the spinal tissue of patients.

Currently, there is no approved diagnostic tool for ALS, resulting in misdiagnosis and significant disease progression before formal diagnosis. The diagnosis of ALS is based thus on a symptom analysis including full medical history of a patient and requires time, because usually a neurologic examination is conducted at regular intervals to assess whether symptoms such as muscle weakness, atrophy of muscles, hyperreflexia, and spasticity are worsening. A non-invasive imaging test for early detection of ALS and for monitoring disease progression would have significant diagnostic and prognostic value thus. To date, there is also no cure for ALS. Only two symptomatic treatments have been approved by the FDA with minor effects on disease progression; and successful attenuation of disease progression requires early and accurate diagnosis to enable patient access to these standard of care therapies.

Therefore, there is an urgent need for diagnostic tools, in particular for tools usable in vivo, which could be used for early detection of misfolded SOD1 as indicator of ALS or for monitoring ALS progression or its treatment.

### SUMMARY OF THE INVENTION

The present invention generally relates to an antibody and equivalent binding molecules, which are conjugated to a detectable label and allow in vivo immunoimaging of misfolded and/or aggregated SOD1 in a subject. The labeled antibody conjugate is particularly useful in a method of diagnosing ALS and/or monitoring the therapy and/or progression of ALS in a subject. The method preferably comprises the detection of misfolded SOD1 by in vivo immunoimaging in the subject. The antibody and equivalent binding molecule specifically bind to misfolded and/or aggregated SOD1 and is conjugated to a detection label. Preferably, the subject is human.

The present invention is based on a recombinant human monoclonal antibody (AP-101 described Maier et al., Sci Transl. Med. (2018):10 (470)) that selectively targets mSOD1, which is coupled to a PET imaging agent, featuring ⁸⁹Zr-DFO-AP-101 radiotracer for PET imaging of mSOD1.

As illustrated in Example 1 and in the preceding Materials and Methods section, DFO-AP-101 was prepared by conjugating AP-101 antibody with p-SCN-Bn desferoxamine p-SCN-Bn desferoxamine (SCN-Bn-DFO) and labelled with ⁸⁹Zr (t_{1/2}=78.41 h). A longitudinal imaging study was performed to identify the optimal mice age and time post administration of ⁸⁹Zr-DFO-AP-101 for the detection mSOD1 aggregation in transgenic (Tg, [B6.Cg-Tg(SOD1*G93A)1Gur/J)]) mice expressing mSOD1 and in wild type (Wt) mice. As control, a subset of mice were co-injected with AP-101 (100 mg/kg) to assess target specificity. PET/CT data were expressed as percent injected dose per gram of tissue (% ID/g).

As demonstrated in Example 4, ⁸⁹Zr-DFO-AP-101 was able to engage mSOD1 aggregates in the spinal cord of Tg mice and imaging was optimal in 126-day-old Tg mice and at day 10 post administration. The number of detected aggregates was more important at day 10 (18) as compared to day−7 (6). All the spots found between the T10 and L2 vertebra. The concentration of ⁸⁹Zr-DFO-AP-101 in the spinal cord and vertebra of the Tg mice significantly exceeded that of the Wt mice (p=0.01). Co-injection with AP-101 considerably reduced the amount of mSOD1 aggregates detected from 67% (6/9) to 15% (5/13). The intensity of the aggregates was also decreased from 8.05±1.30% ID/g to 5.73±0.28 with ⁸⁹Zr-DFO-AP-101 alone or in presence of AP-101 in excess. Blocking with AP-101, significantly reduced the spinal cord (p=0.0002) and vertebra (p<0.0001) uptake of ⁸⁹Zr-DFO-AP-101.

In addition, as shown in Example 7, in context with those experiments it was discovered that misSOD1 can also be found in the bone/joints of the SOD1 animals (PET signal, confirmed by IHC with other misSOD1 antibodies).

In summary, labeled anti-SOD1 antibody conjugates exemplified by the lead ⁸⁹Zr-DFO-AP-101 exhibit unprecedented detection of mSOD1 aggregates in transgenic mice.

Furthermore, as shown in Example 10, the labeled-antibody conjugate of the present can be safely used, its clearance is predominantly physical, with an early urinary excretion. Therefore, this tracer can be safely used in patients. Upcoming data in ALS patients will validate its capacity to measure mSOD1 levels in vivo for diagnosis and treatment monitoring. Accordingly, such labelled antibody conjugates find application in the early diagnostic of ALS and in the monitoring of therapeutic interventions. In addition, such construct but also mSOD1-binding molecules such as anti-SOD1 antibodies in general can be used for targeting SOD1, i.e., misfolded and aggregated SOD1, respectively, at bones and/or joints and may open up a path of potential treatment/improvement of “bone problems” of ALS patients in the future.

Accordingly, the present invention generally relates to antibodies and equivalent SOD1 binding molecules for use in the detection of misfolded and aggregated SOD1 in vivo, preferably immunoimaging of misfolded and/or aggregated SOD1 in a subject. The imaging method for detecting the antibody-label conjugate in the subject is preferably selected from the group comprising or consisting of PET, SPECT, MRI, MPI and optical imaging including fluorescence imaging, or combinations thereof or combinations with CT, such as PET/CT, PET/MRI, PET/fluorescence, SPECT/CT imaging.

The label for the antibody conjugate or equivalent binding molecule is selected depending on the imaging method intended to be used for diagnosing ALS or monitoring drug therapy and/or progression of ALS in a subject. If a use in PET and/or SPECT is intended, preferably the label is a radionuclide or a chemical compound (chelator or prosthetic group) in which one or more atoms have been replaced by a nuclide. If a use only in PET is intended, preferably a nuclide is selected, which emits positrons. If a use only in SPECT is intended, preferably a nuclide is selected which emits gamma rays. If a use in either PET or alternatively in SPECT is intended, preferably the antibody or equivalent binding molecule is conjugated at the same time to a prosthetic group and to a chelator. By this, a PET label, e.g., a nuclide emitting positrons (e.g., F-18) can be conjugated to the prosthetic group and a SPECT label, e.g., a nuclide emitting gamma rays (e.g., Ga-67) can be complexed via the chelator. In one embodiment the antibody or equivalent binding molecule can be conjugated at the same time to a prosthetic group, for labelling with a PET label as exemplarily listed hereinbelow, and to a chelator to bind a SPECT label as exemplarily listed hereinbelow. Inter alia due to several technical advantages of PET over SPECT, according to the present invention preferably PET or PET combined with CT is used in the methods described herein. These advantages of PET over SPECT include a higher photon penetration in PET, which can be combined with depth-dependent attenuation correction. By this, the specificity of the imaging method is increased by reducing the number of false positives. Also, by PET smaller defects than by SPECT can be detected, since PET offers a higher spatial and contrast resolution, again increasing sensitivity. Finally, PET allows a higher temporal resolution, by which the tracer kinetics can be dynamically imaged.

Preferably, the label to be included in the labeled antibody conjugate or labeled equivalent binding molecule is selected from: Fluorine-18 (F-18), Titanium-45 (Ti-45), Manganese-52 (Mn-52), Iron-52 (Fe-52), Kalium-43 (K-43), Scandium-43 (Sc-43), Scandium-44 (Sc-44), Cobalt-57 (Co-57), Copper-60 (Cu-60), Copper-61 (Cu-61), Copper-62 (Cu-62), Copper-64 (Cu-64), Copper-67 (Cu-67), Gallium-67 (Ga-67), Gallium-68 (Ga-68), Bromine-76 (Br-76), Bromine-77 (Br-77), Krypton-81m (Kr-81m), Rubidium-81 (Rb-81), Yttrium-86 (Y-86), Strontium-87m (Sr-87m), Zirconium-89 (Zr-89 or 89Zr), Technetium-99m (Tc-99m), Indium-111 (In-111), Indium-113m (In-113m), Antimony-117 (Sb-117), Iodine-123 (I-123), Iodine-125 (I-125), Caesium-127 (Cs-127), Caesium-129 (Cs-129), Iodine-131 (I-131), Iodine-132 (I-132), Lutetium-177 (Lu-177), Rhenium-186 (Re-186), Quicksilver-197 (Hg-197), Rhenium-188 (Re-188), Lead-203 (Pg-203), Bismuth-206 (Bi-206), Actinium-225 (Ac-225), Radium-225 (Ra-225) and a combination of any two or more thereof.

More preferred, the label is selected from the group comprising or consisting of:
(a) for use in PET from: Fluorine-18 (F-18), Manganese-52 (Mn-52), Copper-64 (Cu-64), Gallium-68 (Ga-68), Antimony-117 (Sb-117), Scandium-43 (Sc-43), Scandium-44 (Sc-44), Titanium-45 (Ti-45), Bromium-76 (Br-76), Yttrium-86 (Y-86), Iodine-124 (I-124) and Zirconium-89 (Zr-89 or ⁸⁹Zr);
(b) for use in SPECT from: Indium-111 (In-111), Technetium-99m (Tc-99m), Gallium-67 (Ga-67), Iodine-123 (I-123), Iodine-131 (I-131), Lutetium-177 and Rhenium-186 (Re-186);
(c) for use in MRI and MPI: magnetic, paramagnetic or superparamagnetic ion complexes or particles, preferably containing Gaddolinum³⁺ (Gd³⁺), Manganese²⁺ (Mn²⁺); Copper²⁺ (Cu²⁺); iron oxide (Fe₃O₄) contrast agents; and
(d) for use in optical imaging including fluorescent imaging from: fluorescein, Cy2, Cy3, Cy3B, Cy3.5, Cy5, Cy5.5, Cy7, Texas Red® (Molecular Probes, Inc., Eugene, OR), AlexaFluor® (Molecular Probes, Inc., Eugene), AlexaFluor 350, AlexaFluor 405, AlexaFluor 430, AlexaFluor 488, AlexaFluor 500, AlexaFluor 532, AlexaFluor 546, AlexaFluor 568, AlexaFluor 594, AlexaFluor 610, AlexaFluor 633, AlexaFluor 647, AlexaFluor 660, AlexaFluor 680, AlexaFluor 700, AlexaFluor 750, BODIPY FL, BODIPY R6G, BODIPY TMR, BOPDIPY 530/550, BODIPY 558/568, BODIPY 564/570, BODIPY 576/589, BODIPY 581/591, BODIPY 630/650, BODIPY 650/665, Rhodamine Green™-X (Molecular Probes, Inc.), Rhodamine Red™-X (Molecular Probes, Inc.), Rhodamine 6G, TMR, TAMRA™ (Applied Biosystems).

More preferred, the detection label is a radionuclide. All radionuclides that may be detected in positron emission tomography (PET) and/or in single photon emission computed tomography (SPECT) can be used according to the present invention and thereby be part of the detection label, however, preferably the physical half-life of a radionuclide should match its intended application, or more specifically, the biologic half-life of the vector, i.e., antibody it is conjugated to.

Radionuclide tracers have an energy of from about 20 to about 4,000 kiloelectronvolts (i.e., keV). For SPECT, either alone or in combination with other imaging methods, preferably radionuclides with an energy of about 30 to about 300 kiloelectronvolts are used. Suitable radionuclide tracers include radionuclides that emit gamma radiation (particularly suitable for SPECT), positrons (particularly suitable for PET), or a combination of gamma radiation and positrons (suitable for either PET or SPECT). In illustrative, non-limiting embodiments, radionuclide tracers have appropriate decay characteristics for optimizing image resolution and quantitative accuracy and/or have appropriate residualization. For example, radionuclide tracers may have a physical half-life (i.e., t½) compatible with the time required for the antibody to achieve optimal specific:non-specific binding ratios. Examples of radionuclide tracers suitable for PET imaging, and suitable for inclusion in a labeled antibody conjugate according to embodiments of the present disclosure, F-18 (t½ about 1.83 hours), Sc-44 (t½ about 3.97 hours), Ti-45 (t½ about 3 hours), Mn-52 (t½ about 5.6 days), Cu-64 (t½ about 12.7 hours), Ga-68 (t½ about 1.13 hours), Br-76 (t½ about 16.2 hours), Y-86 (t½ about 14.7 hours), Zr-89 (t½ about 78.4 hours), I-124 (t½ about 100.3 hours), or a combination of any two or more thereof. Examples of radionuclide tracers suitable for SPECT imaging include Tc-99m (t½ about 6 h hours), In-111 (t½ about 2.80 days), I-123 (t½ about 13.2 hours), I-131 (t½ about 8 days), Lu-177 (t½ about 6.64 days), Re-186 (t½ about 3.7186 days), or a combination of any two or more thereof.

Preferably, according to the invention a radionuclide is used as part of the label with a physical half-life of between about 1 hour to about 10 days. The selection of the label can be also made based on the fact, whether an antibody or an equivalent binding molecule, e.g., an antibody-fragment has to be labeled, preferably matching the biological half-life of the antibody or antibody-fragment or another equivalent binding molecule. If an antibody is to be labeled, preferably a label with a half-life of at least about 1, 2, 3, 4, 5, 6, 7, 8, 9 or about 10 days, more preferred of about 3-10 days, mostly preferred of about 3-7 or 3-5 days is chosen. If an antibody fragment is to be labeled, a label with a shorter half-life can be selected than for an antibody. Preferably, the label for an antibody-fragment has a half-life of at least 10, 9, 8, 7, 6, 5, 4, 3, 2 or 1 days, more preferred of between about 1 hour to 5 days or between about 1 to 48 hours. Accordingly, for labelling antibody-fragments for PET a radionuclide with a short physical half-life is chosen, preferably selected from the group consisting of Cu-64, F-18, Ti-45, Br-76, Y-86, Zr-89 or I-124 or a combination of any two or more thereof. For labelling antibodies for PET preferably a radionuclide with a longer physical half-life is chosen, preferably Zr-89 or I-124. For labelling antibody or antibody fragments for SPECT, preferably a radionucleotide is selected from the group consisting of Tc-99m, In-111, I-123, I-131, Lu-177 and Re-186 or a combination of any two or more thereof.

In one embodiment, for PET, shorter-lived radionuclides such as F-18 and Ga-68 are used for labeling binding molecules intended for same-day imaging after injection. In view of their shorter half-life, in one embodiment preferably antibody fragments that have shorter biological half-lives are used with these labels, such as single domain antibodies, scFvs, or diabodies. Nevertheless, in one embodiments also complete antibodies are labeled with these radionucleotides. In one embodiment, longer-lived radionuclides such as Cu-64, Zr-89 and I-124 are preferably used with a complete antibody or a larger antibody fragment, such as the minibody, for optimal imaging contrast 24-48 hours or later post-injection.

The label of the antibody conjugate of the present invention, and potentially further entities, such as a bifunctional chelator or prosthetic group is also referred to as a tracer, if a radionuclide is used as a detection label as radiotracer in the following. According to the invention, bifunctional chelators are preferably used for labelling of antibodies with radiometals, such as Ga-68, Zr-89, and Cu-64 and prosthetic groups are preferably used for labelling with radio-halides, such as F-18, I 123/124/131.

As illustrated in the Examples, if a radionuclide is used as the label or part of the label according to the present invention, the preferred radionuclide is Zr-89. Accordingly, in a preferred embodiment, the label of the antibody conjugate of the present invention is the radionuclide ⁸⁹Zr.

Preferably the detection label is conjugated to the antibody with a bifunctional chelator or prosthetic group. The bifunctional chelator or prosthetic group can be conjugated in any way resulting in a covalent binding to the antibody. Preferably the chelator or prosthetic group is conjugated randomly on amino acid residues, or on glycan regions of the antibody. Preferably, the bifunctional chelator or prosthetic group is conjugated on a lysine or cysteine residue of the antibody. Mostly preferred the bifunctional chelator or prosthetic group is conjugated randomly on lysine residues of the antibody.

According to the invention generally chelators may be used, which have been shown as usable for complexing a radionuclide, a magnetic, paramagnetic or supermagnetic ion complex or particle, to an antibody. Mostly preferred, the bifunctional chelator comprises p-SCN-Bn desferoxamine (SCN-Bn-DFO).

In a preferred embodiment of the labeled antibody conjugate of the present invention is the antibody AP-101 or a fragment or biological derivative thereof. AP-101 is described in detail in the international patent application published as WO 2012/080518 A1, wherein the antibody is disclosed as antibody NI-204.12G7. The VH and VL-chain sequences of the antibody and its CDRs are indicated in WO 2012/080518 A1 in FIG. 1B and in Table A below. The constant regions of the antibody are preferably derived from human IgG1. However, the human constant domain in the labeled antibody conjugate of the present invention may be also of a different IgG isotype or of a different allotype, for example to avoid or reduce immunogenicity which can happen as a result of allo-immunization; see, e.g., for review Jefferis and Lefranc, MAbs 1 (2009), 332-338. The antibody AP-101 and methods for its production are also described in detail in the international patent application published as WO 2024/042250 A1. The entire disclosure content of WO 2012/080518 A1 and WO 2024/042250 A1 and in particular the passages explicitly cited herein are incorporated herein by reference,

The antibody portion of the labeled antibody conjugate of the present invention can be glycosylated or deglycosylated. In one preferred embodiment the antibody is glycosylated, preferably wherein the antibody is produced in a CHO cell. CHO cells are the most widely used mammalian cells for the production of recombinant monoclonal antibodies due to their ability to perform post-translational modifications (PTMs) on the antibody molecules, which typically take place in the human body as well. Through genetic manipulation by mutagenesis, different CHO daughter cells with improved qualities have been established. Among those variants are CHO-K1, CHO-S, CHO-DXB11 and CHO-DG44. Therefore, in one embodiment, the antibody AP-101 is produced in CHO cells, more preferred in CHO-K1 cells (ATCC No. CCL 61). Preferably, after production, the AP-101 antibody is purified from the cell culture medium for further use. In another embodiment, the antibody is deglycosylated. Preferably, by the deglycosylation the N-glycans were removed from the antibody. Deglycosylation can be obtained by different methods according to the invention. Fc-glycans can be deglycosylated, e.g., by an enzymatic treatment with an endoglycosidase acting on complex type N-glycans or the antibody can be produced in a bacterial host such as E. coli, wherein no glycosylation has occurred.

As described in Example 7 at pages 104 and 105 and in particular in Table 18 at page 105 of WO 2024/042250 A1, the major PTMs that have been identified in antibody AP-101 are the modification in the HC of glutamine at the N-terminus to pyro-glutamic acid, the loss of C-terminal lysine, and N-glycosylation. In this context, the N-glycosylation site was identified at position 303 (HC N303). Thus, in one embodiment, the anti-SODl antibody for use in accordance with the present invention has lost the C-terminal lysine, i.e., the antibody has undergone C-terminal lysine clipping. In particular, the C-terminal lysine is chopped of the heavy chain of the antibody, preferably of each heavy chain of the antibody. In addition, or alternatively, the glutamine at the N-terminal is modified as pyro-glutamic acid, i.e., the antibody has undergone N-terminal glutaminyl cyclization. In addition, or alternatively, the antibody is glycosylated, in particular N-glycosylated. More particularly, the heavy chain of the antibody is glycosylated and even more particularly N303 of the heavy chain.

In a preferred embodiment, the anti-SODl antibody, preferably the AP-101 antibody for use in accordance with the present invention lacks the C-terminal cysteine, has a modified glutamine at the N-terminal as pyroglutamic acid and comprises at least one N-glycosylation site, or as used in a pharmaceutical formulation, preferably most of the antibody species present in the formulation have the mentioned modifications.

The present invention also generally relates to the labeled antibody conjugate as disclosed herein, wherein the labeled-antibody conjugate is suitable for in vivo immunoimaging of misfolded SOD1 in a subject. The present invention also relates to the labeled antibody conjugate of the present invention disclosed herein for use in a method of diagnosing ALS and monitoring drug therapy and/or progression of ALS in a subject, wherein the method comprises in vivo immunoimaging in a subject. Preferably, the imaging of the labeled antibody conjugate in the subject comprises positron emission tomography (PET) imaging, single photon emission computed tomography (SPECT) imaging, magnetic resonance imaging (MRI), magnetic particle imaging (MPI), or PET or SPECT in combination with computed tomography (CT), MRI or MPI.

In a further aspect the present invention generally relates to a drug for use in the treatment of ALS in patient which has been diagnosed for suffering or developing ALS and/or is monitored for efficacy of drug therapy and/or disease progression of ALS by a method as defined herein. Preferably the drug is selected from Riluzole (Rilutek®, Tigultik, Exservan™), Edaravone (Radicava®), Tofersen (BIIB067), AP-101, dextromethorphan HBr and quinidine sulfate (Nuedexta®), Reldesemtiv (CY5031), Arimoclomol, Levosimendan, Fasudil, pyrimethamine, (Daraprim™), rapamycin, baclofen (Gablofen®, Kemstro®, Lioresal®), diazepam (Diastat®, Valium®), amitriptyline (Elavil®), trihexyphenidyl, Scopoderm® (scopolamine patch), glycopyrrolate (Robinul®), Ravulizumab (also known as BNJ441, ALXN1210, or Ultomiris®) and Eculizumab (also known as Soliris®).

Preferably the labeled antibody conjugate is ⁸⁹Zr-DFO-AP-101 and comprises
(a) AP-101 as the antibody that specifically binds to misfolded SOD1;
(b) p-SCN-Bn desferoxamine (SCN-Bn-DFO) as the bifunctional chelator, and
(c) ⁸⁹Zr as the radionuclide.

In a preferred embodiment, the labeled antibody conjugate of the invention is glycosylated. In another preferred embodiment, the labeled antibody conjugate as defined hereinabove is further characterized by the fact that the AP-101 antibody is deglycosylated. Such a deglycosylated antibody is interchangeably designated as deglycosylated ⁸⁹Zr-DFO-AP-101 or as Degly-⁸⁹Zr-DFO-AP-101. When labeled with Cu-64, such a deglocosylated antibody is interchangeably designated as degylcosylated ⁶⁴Cu-NOTA-AP-101 or as Degly-⁶⁴Cu-NOTA-AP-101.

Furthermore, the present invention provides and relates to an antibody conjugate intermediate, i.e., the antibody portion wherein the antibody that specifically binds to misfolded SOD1 comprises a bifunctional chelator, preferably wherein the chelator isp-SCN-Bn desferoxamine (SCN-Bn-DFO). Such an antibody conjugate intermediate is particularly useful for preparation of the labeled antibody conjugate of the present invention in that it already comprises the bifunctional chelator conjugated to the antibody and the option that a label can, depending on the intended use, be selected and conjugated thereto. Specific labels which can be conjugated thereto, depending on the imaging technique in which it is intended to use the then conjugate, i.e., usable in PET, SPECT, MRI or MPI, PET/CT, PET/MRI, SPECT/CT or SPECT/MRI are listed above.

The present invention also provides an imaging composition comprising the antibody or antibody conjugate as defined hereinabove and further comprising pharmaceutical acceptable excipients, preferably NaCl and gentisic acid, preferably at a pH of 4.5 to 8.0. Preferably, the imaging composition is a 10 mL solution constituted of NaCl (0.9%) and gentisic acid (10 mg) in a sealed vial, containing about 10 mg labeled-antibody conjugate and between about 300 and 500 MBq, preferably wherein the amount of labeled-antibody conjugate vs. unlabeled antibody is greater than 97% and/or wherein the labeled-antibody conjugate has an apparent specific activity of about 0.656 GBq/mg, optionally wherein the injected solution can be diluted in NaCl prior injection. When preparing a corresponding imaging composition with deglycosylated ⁸⁹Zr-DFO-AP-101, the labeled-deglycosylated-antibody conjugate had an apparent specific activity of about 0.46 MBq/μg.

The present invention further provides a diagnostic composition or kit comprising the labeled antibody conjugate or antibody conjugate intermediate as defined herein (i.e., potentially comprising the deglycosylated or the intermediate forms comprising the chelator), optionally further comprising a detection label, preferably a radionuclide, preferably wherein the radionuclide is Zr-89; or the imaging composition as defined in the paragraph above; and optionally instructions for use in a method of diagnosing amyotrophic lateral sclerosis (ALS) or monitoring drug therapy and/or progression of ALS in a subject as defined in hereinabove.

Furthermore, the present invention also relates to a method of in vivo immunoimaging of superoxide dismutase (SOD1) useful as a marker of amyotrophic lateral sclerosis (ALS) or a marker for efficacy of drug therapy of ALS in subject, comprising
(a) administering a labeled-antibody conjugate as defined herein above and below to a subject; and
(b) detecting the presence of the labeled-antibody conjugate in the subject in vivo by imaging. The imaging is preferably performed by a method selected from PET, SPECT, MRI and MPI, optical imaging including fluorescence imaging, or from a combination such as PET/CT, SPECT/CT, PET/MRI and SPECT/MRI.

Preferably the method of in vivo immunoimaging of superoxide dismutase (SOD1) is provided wherein the method comprises conducting a radiographic imaging method on the subject after administration of the labeled-antibody conjugate; and making a radiographic image of the subject for detecting the labeled-antibody conjugate; wherein the radiographic image is diagnostic for the presence of misfolded and/or aggregated SOD1 in the subject.

Furthermore, a method is provided by the present invention of generating an image of a human body, preferably the human body of an ALS patient comprising administering a labeled-antibody conjugate as defined herein to the human body and generating and image of at least a part of the human body to which the labeled-antibody conjugate has distributed.

Preferably, the method of in vivo immunoimaging of superoxide dismutase (SOD1) or the method of generating an image of a human body is provided, wherein the labeled-antibody conjugate is present in or provided by the imaging composition comprising the antibody or antibody conjugate as defined hereinabove.

Preferably one of the foregoing methods is provided, wherein the method comprises contacting cells, tissue or an organ with the labeled antibody conjugate, wherein the labeled antibody conjugate is administered with a target dose in the range of 10-100 MBq; and making a radiographic image for detecting the labeled antibody conjugate. Particularly preferred, the labeled antibody conjugate is administered in such a method with a target dose in the range of about 40 MBq±10%.

Furthermore, in any of the foregoing methods, preferably the labeled antibody conjugate is administered with a target dose, which is significantly lower than the therapeutic dose of the antibody. Preferably the foregoing methods are provided, wherein the labeled-antibody conjugate is administered with a target dose, wherein the antibody protein amount is less than 2 mg. The labeled-antibody conjugate may be administered in these methods using different administration routes, however, preferably the labeled-antibody conjugate is administered via intravenous injection. The final volume for injection in these methods is preferably based on the HEPES or gentisic acid contents for which the specifications are ≤200 μg/injection and 5 mg/injection respectively. The labeled-antibody conjugate may be administered in several doses in these methods, however, preferably it is administered at a single dose. A brief time after administration of the labeled-antibody conjugate the aforementioned methods for immunoimaging or image generation can be performed. Preferably these methods are performed at a time after administration of the labeled-antibody conjugate, wherein the time is selected from the group consisting of about 1 hour, 2 hours, 3 hours and 24 hours, mostly preferred wherein the method is performed 2 hours after administration (intravenous injection in the preferred embodiment).

Since the half-life of AP-101 in serum is about 9.5 days and also the labelled antibody conjugate is quite stable, as could be seen on pictures obtained in animals 3 and 10 days after injection of the labelled antibody conjugate (see, e.g., FIGS. 3 and 8), in one preferred embodiment, the imaging methods described above are performed 1, 3, 7 and/or 10 days after the administration (post the intravenous injection).

Furthermore, the forementioned methods are provided, wherein the imaging method is selected from the group consisting of planar imaging, positron emission tomography (PET), single photon computed tomography (SPECT) and combinations thereof with computed tomography (CT).

Preferably, said methods are provided, wherein the imaging is performed for a region of interest (ROI) selected from the group of organs/tissues consisting of liver, kidney, blood/heart (left ventricular chamber), spleen, muscle, subcutaneous fat, red-marrow, bone, joint, cartilage tissue. Mostly preferred said methods are provided, wherein the imaging is performed for a ROI as also indicated in the context of the experimental results shown herein, e.g., in FIGS. 1-4, 10, 13, 17 and 21, i.e., in the spinal cord and regarding the bones in the vertebra bones.

In a further embodiment the forementioned methods are provided, wherein the labeled-antibody conjugate is administered to predict effectiveness of a therapy drug for treating a disease caused by and/or associated with the presence of misfolded and/or aggregated SOD1. Preferably, the therapy drug is selected from Riluzole (Rilutek®, Tigultik, Exservan™), Edaravone (Radicava®), Tofersen (B11B067), AP-101, dextromethorphan HBr and quinidine sulfate (Nuedexta®), Reldesemtiv (CY5031), Arimoclomol, Levosimendan, Fasudil, pyrimethamine, (Daraprim™), rapamycin, baclofen (Gablofen®, Kemstro®, Lioresal®), diazepam (Diastat®, Valium®), amitriptyline (Elavil®), trihexyphenidyl, Scopoderm® (scopolamine patch), glycopyrrolate (Robinul®), Ravulizumab (also known as BNJ441, ALXN1210, or Ultomiris®) and Eculizumab (also known as Soliris®).

Furthermore the forementioned methods of in vivo immunoimaging of superoxide dismutase (SOD1) and generation of an image of the human body are provided, wherein the labeled-antibody conjugate is administered to diagnose ALS or monitor the effect of the therapeutic treatment of ALS, the method comprising conducting a radiographic imaging method on the subject after administration of the labeled-antibody conjugate; and making a radiographic image of the subject for detecting the labeled-antibody conjugate; wherein the radiographic image is diagnostic for ALS and the effectiveness of the therapeutic treatment of ALS, respectively.

In a further embodiment, the present invention relates to an anti-SOD1 antibody or an equivalent SOD1 binding molecule, for example an antibody fragment as defined further below, for use in targeting misfolded and/or aggregated SOD1 in bone and/or joints of a subject. Herein, under a bone and/or joint related disease a disease is understood which impairs the structure and/or function of bones and/or joints in a subject. Preferably, the antibody or equivalent binding molecule is capable of binding to misfolded and/or aggregated SOD1. Preferably the anti-SOD1 antibody or equivalent SOD1 binding molecule is the antibody AP-101, a SOD1-binding fragment thereof or a labeled antibody conjugate as described herein. Preferably the subject is a mammal, more preferably the subject is a human. In a preferred embodiment the disease is ALS. Furthermore, the present invention also relates to ⁸⁹Zr or ⁸⁹Zr-DFO for use in immunoimaging of misfolded and/or aggregated SOD1 in a subject, preferably for use in the labeled antibody conjugate as disclosed herein and/or for use in early diagnostic of ALS and/or in the monitoring of therapeutic interventions of ALS. Furthermore, in one embodiment a pharmaceutical composition is provided, comprising the anti-SOD1 antibody or an equivalent binding molecule, the labeled antibody conjugate or equivalent SOD1 binding labeled molecule and a pharmaceutically acceptable carrier for use in targeting misfolded and/or aggregated SOD1 in bone and/or joints of a target.

In a further embodiment, the present invention also provides a use of an antibody or labeled antibody conjugate as defined above for the manufacture of an imaging composition for use in one of the methods as defined hereinabove.

In another embodiment, the present invention further provides a method for preparation of a solution comprising the ⁸⁹Zr-DFO-AP-101 antibody conjugate as defined above comprising the steps of:
(a) combining a ⁸⁹Zr-chloride solution with DFO-AP-101 to obtain a reaction solution;
(b) warming the reaction solution to a temperature of about 35° C. to about 42° C., preferably of about 37° C. for a period of about 15 minutes to about 90 minutes, preferably for a period of about 45 minutes; and
(e) recovering [⁸⁹Zr]Zr-DFO-AP-101 from the reaction solution into a recovered solution.

Preferably the method further comprises one or both of the following steps:
(f) adjustment of the [⁸⁹Zr]Zr-DFO-AP-101 recovery solution to a final volume by adding saline, preferably wherein the final volume is 10 mL; and/or
(g) filtering of the [⁸⁹Zr]Zr-DFO-AP-101 recovery solution.

In step (a) the ⁸⁹Zr-chloride solution is preferably adjusted to a pH of 6.5-8.0. The adjustment is preferably performed by adding a sodium bicarbonate solution and HEPES buffer solution to the ⁸⁹Zr-chloride solution. Furthermore, the step of recovering of [⁸⁹Zr]Zr-DFO-AP-101 is preferably performed on a column comprising a gel filtration resin comprising the following steps:
(c) conditioning a gel-filtration resin in a column with 10 mL saline 0.9%, preferably at 1.5 mL/min; wherein preferably this step is repeated 3 times. The conditioning is preferably performed during the warming of the reaction solution.
(d) transferring the reaction solution of (b) with 4 mL of saline to the conditioned column of (c);
(e) recovering [⁸⁹Zr]Zr-DFO-AP-101 from the reaction solution into a recovered solution from the column.

Accordingly, in a preferred embodiment, the above method is performed comprising the following steps:
(a) combining an adjusted to a pH of 6.5-8.0 ⁸⁹Zr-chloride solution with DFO-AP-101 to obtain a reaction solution;
(b) warming the reaction solution to a temperature of about 37° C. for a period of about 45 minutes;
(c) conditioning a gel-filtration resin in a column with 10 mL saline 0.9%, preferably at 1.5 mL/min; preferably wherein this step is repeated 3 times;
(d) transferring the reaction solution of (b) with 4 mL of saline to the conditioned column of (c);
(e) recovering [⁸⁹Zr]Zr-DFO-AP-101 from the column as a recovery solution;
(f) adjustment of the [⁸⁹Zr]Zr-DFO-AP-101 recovery solution to the final volume by adding saline, preferably wherein the final volume is 10 mL; and preferably
(g) filtering of the [⁸⁹Zr]Zr-DFO-AP-101 solution.

Furthermore, this preparation method is provided wherein the ⁸⁹Zr-chlorid solution of step (a) is provided by the following steps:
(a1) providing a solution of ⁸⁹Zr-oxalate onto a quaternary methyl ammonium (QMA) anion exchange column;
(a2) adding a solution comprising HCl to the QMA anion exchange column; and
(a3) collecting the ⁸⁹Zr-chloride solution eluting from the column.

Preferably the above defined preparation method is provided, wherein the method further comprises the step of.
(c) formulation of the filtered [⁸⁹Zr]Zr-DFO-AP-101 solution obtained from step (g) into a composition by addition of 0.9% saline and 10 mg of gentisic acid.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. The disclosure content of all publications, patent applications, patents, and other references mentioned herein are incorporated by reference in their entirety. The materials, methods, and examples are illustrative only and not intended to be limiting.

Other features and advantages of the invention will be apparent from the following detailed description and from the claims.

For the avoidance of any doubt it is emphasized that the expressions “in some embodiments”, “in a certain embodiment”, “in some aspects”, “in certain aspects”, “in a certain aspect”, “in a further aspect”, “in certain instances”, “in some instances”, “in a further embodiment”, “in one embodiment” and the like are used and meant such that any of the embodiments described therein are to be read with a mind to combine each of the features of those embodiments and that the disclosure has to be treated in the same way as if the combination of the features of those embodiments would be spelled out in one embodiment. The same is true for any combination of embodiments and features of the appended claims and illustrated in the Examples, which are also intended to be combined with features from corresponding embodiments disclosed in the description, wherein only for the sake of consistency and conciseness the embodiments are characterized by dependencies while in fact each embodiment and combination of features, which could be construed due to the (multiple) dependencies must be seen to be literally disclosed and not considered as a selection among different choices.

### BRIEF DESCRIPTION OF THE DRAWINGS

FIG. 1: Steps in mSOD1 aggregate identification and processing. (A) The spots are visually identified on the PET images and a region of interest (ROI) including the whole spot is drawn. (B) Spots which are close to the area of spilling from radiotracer in the anterior vertebra bone are not retained for analysis. (C) Within the same mouse, a similar ROI is traced on an area of the spinal cord devoid of mSOD1 to obtain an internal reference. (D) An external reference is obtained from the spinal cord of the control mice either by drawing a discrete ROI at the level corresponding to the Tg mSOD1 spot or by tracing a ROI including all the control mice spinal cord (profile). (E) The amount of radiotracer in mSOD1 spots in unit of % ID/g is reported along the other observed parameters. (F) Overview table about the spots counted in the mice depending on the age of the animals. (G) Visualization of the spot intensity in comparison to the animals' age. Older animals show more spots/animal.

FIG. 2: Pictures from SOD1 G93A transgenic ALS mice (A) Sagittal PET images showing profile ROI traced on the spinal cord and anterior vertebra bone area. (B) Transaxial PET image showing the ROI traced on the spinal cord and anterior vertebra. (C) Transaxial CT image showing the ROI traced on the spinal cord.

FIG. 3 PET images taken at day−10 following iv administration of ⁸⁹Zr-DFO-AP-101 to 4 months old SOD1 G93A transgenic (Tg) mice, showing the presence of radiotracer in the head of the femur (Top) and in the spleen (Bottom). The arrows point to ROI selected on the head of the femur and the spleen to quantify their radiotracer content. The signal within the spinal cord increased over the time from day 3 to day 10 (not shown).

FIG. 3-1: PET images showing the uptake of ⁸⁹Zr-DFO-AP-101 in bone (ventral vertebra, head of the femur) and in the spleen in absence [(A) Wt and (C) Tg] and presence [(B) Wt and (D) Tg] of co-injected AP-101 (100 mg/kg) at day 7. Blocking with AP-101 reduced the uptakes of ⁸⁹Zr-DFO-AP-101 in these tissues. The presence of ⁸⁹Zr-DFO-AP-101 in the vertebra bone at time could be intense enough to spill-in the spinal cord making the quantification of the amount of radiotracer in the mSOD-1 spots unreliable. To reduce this effect all images were corrected for spill in and recovery coefficient using a well-established mathematical models as described in Émilie Gaudin et al., Phys. Med. Biol. 66 (2021) 065019, which content is incorporated hereby in its entirety. The uptake of ⁸⁹Zr-DFO-AP-101 in bone and spleen was found to be specific as it was blocked by co-injection of unlabeled AP-101. AP-101 would not only bind to mSOD1 but also to Fc receptor, to reduce Fc binding, Deglycosylated ⁸⁹Zr-DFO AP-101 was synthesized and evaluated. Large effect of blocking in wildtype mice show large contribution of non-misfolded SOD1 signal.

FIG. 3-2: In the scattergrams (E-H) quantification of PET signal (% ID/g; individual mice with mean value) is provided in the ROI of the spinal cord (E), vertebra bone (F), head of the femur (G) and spleen (H) in SOD1 G93A transgenic and Wildtype mice after injection of ⁸⁹Zr-DFO-α-miSOD1 along with blocking (100 mg/kg unlabeled α-miSOD1) and deglycosylated ⁸⁹Zr-DFO-α-miSOD1. *** p<0.001, **** p<0.0001 (One Way ANOVA, Dunnett's multiple comparison test).

FIG. 4 Radioactivity measured within the spinal cord was corrected from Spill-in of ⁸⁹Zr-tracer present in the ventral vertebra bone and from the partial volume effect using the recovery coefficient RC.

FIG. 5 Partial volume effect (PVE) correction using Ultra-Micro Hot Spot phantom. (A) Transverse view of a 0.3 mm slice obtained from the Ultra-Micro hot spot phantom and line profiles across the hot spots at the positions indicated by the yellow lines. (B) Mean activity over the hot spots (left y-axis) and recovery coefficients obtained by dividing the hot spot mean activity by the largest hot spot max activity (right y-axis) (Gaudin et al. 2021).

FIG. 6 Binding of ^{nat}Zr-DFO-AP-101 to A) denatured SOD1 and B) native SOD1. In a direct enzyme-linked immunosorbent assay (ELISA) non-modified α-miSOD1 (solid grey line with closed circles), DFO labeled ^{nat}Zr-DFO-AP-101 (solid black line with open triangles) and deglycosylated ^{nat}Zr-DFO-AP-101 (dashed line with open squares) bound with high affinity to denatured human SOD1 (A) with EC₅₀ values of 0.2, 0.3 and 2.4 nM respectively, while showing only minimal binding to the physiological SOD1 dimers (B).

FIG. 7 Characterization of Degly-DFO-AP-101. Characterization and comparison of binding properties of DFO-conjugated, deglycosylated to non-modified AP-101. (A) HPLC tracing of purified deglycosylated DFO-AP-101 (1), DFO-α-miSOD1 (2) and deglycosylated DFO-AP-101 before purification (3) showing the presence of free glycans (4) after 2 h incubation with ENDO-S2. (B) SDS-PAGE/Comassie of reduced AP-101 (1) and deglycosylated α-miSOD1 with ENDO-S2 after 2 h incubation (2)

FIG. 8 Residual radiotracer content in non-target organs ex vivo after perfusion of the mice. The residual radioactivity (expressed as percent of the injected dose per gram of tissues; % ID/g) of main non-target tissue 10 days after injection of ⁸⁹Zr-DFO-AP-101, after blocking with 100 mg/kg unlabeled α-miSOD1 (Blocked) or degly ⁸⁹Zr-DFO-AP-101 (Deglycosylated) for SOD1 G93A transgenic (Tg; filled symbols) and wildtype (Wt; open symbols)) mice measured in total blood (A) or after perfusion of the mice in spleen (B), liver (C), tibia bone (D), lungs (E) and kidneys (F). Mean % ID/g±SEM; ns not significant, ** p<0.01, *** p<0.001, ****p<0.0001 (One Way ANOVA; Dunnett's post-hoc)

FIG. 9 SDS-PAGE of Degly-⁸⁹Zr-DFO-AP-101 formulated in PBS alone (A) or in presence of GA (B). *Monitored under reducing conditions.

FIG. 10 Illustration of experimental groups/conditions described herein: distribution of the transgenic (Tg or ALS) and wild type (Wt or CTL) mice within the different experimental groups. The study was performed in Tg [B6.Cg-Tg(SOD1*G93A)1Gur/J)] mice expressing the misfolded form of SOD1 and their control Wt [C56BL/6J] mice. The study was undertaken to select the optimal mice age and time post administration of ⁸⁹Zr-DFO-AP-101 to carry PET Imaging. Following this study, it was decided to use mice older than 126 days and to image at days 7 and 10 post administration of the radiotracer. The feasibility of using ⁸⁹Zr-DFO-AP-101 for the detection of mSOD1 aggregated in the spinal cord was evaluated by blocking with unlabeled AP-101 to establish the specificity of ⁸⁹Zr-DFO-AP-101 for mSOD1.

Deglycosylation was also explored as a mean to evaluate the impact of Fc binding of ⁸⁹Zr-DFO-AP-101. (A) Previously completed longitudinal study aimed at selecting the best age and day post administration of the radiotracer for PET imaging. (B) Current study assessing the feasibility of using ⁸⁹Zr-DFO-AP-101 for PET imaging of misfolded SOD1 (mSOD1).

FIG. 11 Scattergram comparing the weight of 126 days old Tg and Wt mice and descriptive statistics. The Tg and Wt mice weights were compared using an unpaired Two-tails Student t test.

FIG. 12 Scattergram comparing the dose per gram of radiotracer administrated to 126 days old Tg and Wt mice and descriptive statistics. The Tg and Wt mice received doses were compared using an unpaired Two-tails Student t test.

FIG. 13 Distribution of mSOD1 spots within the mice spinal cord. The spots detected with either ⁸⁹Zr-DFO-AP-101 and its deglycosylated analog are represented in the left MIB PET/CT image. The false (+) spot, which includes control (Wt) mice and blocked mice (Tg and Wt) presenting valid spots are also included in the figure. Data from the table are also visualized in FIG. 21 I-J.

FIG. 14 Bar-graph comparing the frequency of mSOD1 spot detection (% mice with spots), average intensity of the detected mSOD1 spots and the mSOD1 spot to internal reference ratio. The error bar stands for the standard deviation of the mean (sdm). The number on top of the bars represents the number of mice in the group. The error bars represent the sample standard deviation of the mean. The “p” values were obtained using a Two-tails Student-t test.

FIG. 15 The internal reference values measured on the spinal cord of Tg mice receiving the deglycosylated analog were significantly higher than those measured in the Tg mice receiving ⁸⁹Zr-DFO-AP-101 (Two-tails Student-t test). This higher background value will contribute to increasing the % ID/g value of the mSOD1 spots from the Degly-⁸⁹Zr-DFO-AP-101 mice.

FIG. 16 Whole body maximal intensity projection PET/CT images showing the biodistribution of ⁸⁹Zr-AP-(A, D) in 4 months old SOD1 G93A transgenic (Tg, A, B, C) and control wildtype (D, E, F) mice along with blocking (B, E; 100 mg/kg unlabeled AP-101 and deglycosylation conditions (C, F) in liver, spleen, spinal cord, ventral vertebra bone and head of the femur. (G) annotation of positive areas.

FIG. 17 Scattergram comparing the spinal cord uptake (A), the vertebra ventral bone uptake (B) and the spinal cord to vertebra ventral bone uptake ratio (C) of ⁸⁹Zr-DFO-AP-101 to that of the blocked conditions and Degly-⁸⁹Zr-DFO-AP-101. The % ID values were obtained from profile ROI traced on the spinal cord. For each condition, the % ID/g values measured in the Tg (orange; first scattergram in each figure) and Wt (blue; second scattergram in each figure) mice were compared using a Two-tail Student-t test, the “p” values are given at the bottom of the graph. The % ID/g values for the Tg mice receiving ⁸⁹Zr-DFO-AP-101 were compared to those of the blocked and deglycosylated groups using a One-way ANOVA and a Dunnett's multiple comparison test to compare the values obtained with ⁸⁹Zr-DFO-AP-101 with the blocked and deglycosylated conditions, (dotted lines and “p” values).

FIG. 18 Correlation between the % ID/g values measured by PET imaging or by biodistribution. Except for the spinal cord where the tissue content in radiotracer was low. There was a significant correlation between the % ID/g values measured by PET imaging or biodistribution.

FIG. 19 Compilation of mSOD1 identification data at day−10 post administration of the radiotracer and structure of ⁸⁹Zr-DFO-AP-101 and of its deglycosylated analog.

FIG. 20 ⁸⁹Zr-DFO-AP-101 (B) binds in a similar manner to soluble misfolded SOD1 compared to non-modified AP-101 (A) in Bio-Layer Interferometry (BLI). Absolute signal and K_{D} is slightly reduced for the Chelator-modified AP-101 suggesting some minor changes.

FIG. 21 Localization and quantification of PET positive misSOD1 aggregates using ⁸⁹Zr-DFO-AP-101 and direct binding of DFO-AP-101 to misSOD1 aggregates using immunohistochemistry on spinal cord sections. (A) Transaxial (left, circle) and sagittal (right) PET/CT-image of a PET positive spot identified within the spinal cord (arrow). (B) Dorsal view of the CT image showing the vertebral column of a SOD1 G93A transgenic mice and the vertebra classification (V, left, T11-L6) and the corresponding spinal cord region applied (Sc, right, L1-S1 based on Harrison, 2013). (C) Bar graph showing the number of misSOD1 aggregates identified on the PET images at each vertebra level for immuno-PET with ⁸⁹Zr-DFO-AP-1011 (dark grey bar) and its deglycosylated analog (light grey bar). (D) Percent of SOD1 G93A transgenic (Tg, dark grey) and Wildtype (Wt, light grey) mice presenting valid spots for ⁸⁹Zr-DFO-AP-101, ⁸⁹Zr-DFO-AP-101 co-injected with 100 mg/kg of AP-101 (Blocked) and deglycosylated ⁸⁹Zr-DFO-AP-101 respectively. The total number of spots per total number of mice imaged is presented above each column. (E) Quantification of radiotracer in the misSOD1 aggregates (% ID/g) for transgenic (black) and Wt mice (open symbol) with ⁸⁹Zr-DFO-AP-101, blocked or deglycosylated analogs. Mean±SEM of all spots and the number of mice imaged for each group is indicated. (**** p<0.0001, One Way ANOVA, Dunnett's multiple comparison test for SOD1 G93A mice). (F) Ratio of the misSOD1 aggregate radiotracer content to internal background (internal reference ROI traced on area of the spinal cord of the same mouse devoid of a misSOD1 spots) for SOD1 G93A transgenic (black symbol) and wildtype mice (open samples) with ⁸⁹Zr-DFO-AP-101, blocked and deglycosylated conditions. Mean±SEM of all spots. (G) Transaxial histological section through the spinal cord of a SOD1 G93A transgenic mouse showing the misSOD1 pathology labelling using immunohistochemistry with α-miSOD1 at a location where positive PET-spots has been identified (see insert) after injection of ⁸⁹Zr-DFO-AP-101.

FIG. 22 Misfolded SOD1 detection in the bone of SOD1 G93A (A) and in SOD1 G37R (B) transgenic mice with AP-101 and commercial misSOD1 antibody (B8H10 from MediMaps). While no immunoreactivity could by detected with the isotype control antibody (1 nM isotype Ctrl row)) or in wildtype mice, misSOD1 agglomerates (exemplarily marked with black arrows) can be observed in bone and cartilage tissue of vertebrae, and joint area of two different SOD1 transgenic mice, G93A and G37R.

FIG. 23 Biodistribution of ⁸⁹Zr-DFO-AP-101 in A) female and B) male C57BL6 mice at various time points. (injected activity: 0.76-0.97 MBq)

FIG. 24 HPLC profile of DFO-AP-101 after its purification (A; 2020-10-27); and 2 years latter (B, 2022-09-14)

FIG. 25 Size-exclusion chromatography analysis of DFO-AP-101 (1-blue line, standard), purified ⁸⁹Zr-DFO-AP-101 (2-red line) and ⁸⁹Zr-DFO-AP-101 after 29 h in saline (3-black line). The analysis was done using a PRLP-S 10 μm, 1000A 4.6×250 mm column.

FIGS. 26A-26B Percent injected dose of five healthy volunteers (2 male in FIG. 26A and 3 female in FIG. 26B) aged 63±8 years-old with 41±4 MBq of ⁸⁹Zr-DFO-AP-101. Segmentation was performed from a 45 min whole body PET/CT at two hours post injection and at days 1, 3, 7 and 10. All PET scans were performed on a Biograph Vision 600 scanner (Siemens, Erlangen, Germany). Reconstructed from the list-mode acquisition use an iterative algorithm with point spread function and time of flight modeling. The whole-body images had a matrix size of 440×440 voxels with an isotropic voxel size of 3.3 mm³. 5 diagrams in graph as follows: Spleen—dark blue line (marked with “a”, at time 0 third line from the top in both figures); Kidney—orange line (marked with “b”, at time 0 fourth line from the top in both figures); Liver—grey line (marked with “c”, at time 0 top line in both figures); Hearth—yellow line (marked with “d”, at time 0 second line from the top in both figures); Bone—light blue line (marked with “e”, at time 0 lowest line in both figures).

FIG. 27 A 62 years old (yo) female of 79 Kg is intravenous injected with 35 MBq of ⁸⁹Zr-DFO-AP-101. A 45 min whole body PET/CT acquisition is performed at two hours(A) post injection and at days 1(B), 3(C), 7(D) and 10(E). All PET scans were performed on a Biograph Vision 600 scanner (Siemens, Erlangen, Germany) with a 26 cm axial field of view. Reconstructed from the list-mode acquisition use an iterative algorithm with point spread function and time of flight modeling. The whole-body images had a matrix size of 440×440 voxels with an isotropic voxel size of 3.3 mm³.

### DETAILED DESCRIPTION OF THE INVENTION

The present invention relates to the embodiments as characterized in the claims, disclosed in the description and illustrated in the Examples and Figures further below.

In particular, the present invention relates to antibodies and antibody conjugates, respectively, which are useful in the diagnosis and monitoring treatment/progression of diseases due to or associated with misfolded SOD1, in particular neurogenerative diseases such as amyotrophic lateral sclerosis (ALS).

The present invention is based on the concept of using an anti-SOD1 antibody that selectively binds to misfolded and/or aggregated SOD1 for targeting such SOD1 species in human tissue of ALS patients and visualize target engagement by a detectable label in vivo. It was hoped that by means of such labeled anti-SOD1 antibody conjugate the severity and progress of the disease could be determined qualitatively and quantitatively.

Tracers that can facilitate ALS diagnosis, aid in prognosis, and measure drug pharmacodynamics are needed to accelerate therapeutic development for patients with ALS and identify patients at an early stage of the disease. Positron emission tomography (PET) imaging with an appropriate radiotracer has great potential for ALS given that it would permit visualization of central nervous system (CNS) pathology in individuals living with the disease.

Recently, a recombinant human monoclonal antibody, with selective binding to human misfolded SOD1 (mSOD1) has been investigated, designated AP-101, which has been originally disclosed in international application WO 2012/080518 A1 and further characterized in Maier et al., Sci. Transl. Med. 10 (2018)(470) doi: 10.1126/scitranslmed.aah3924. Besides its disease-modifying properties, recent experiments suggest a high specificity of this anti-SOD1 antibody for mSOD1.

The AP-101 antibody has been shown to identify mSOD1 in post-mortem spinal cord tissue of both familial and sporadic ALS patients regardless of their SOD1 genotype (Maier et al. 2018). To further confirm this hypothesis the present inventors developed a proprietary sensitive assay for the detection of mSOD1 in biological fluids, specifically cerebral spinal fluid (CSF), from ALS patients (ALS-Detect ELISA). According to preliminary data from a controlled collection dataset generated to support the assay development, it was possible to determine that mSOD1 was present in the CSF of a large majority of SOD1 mutant FALS patients tested. These results suggest that screening the CSF of patients with known disease is useful for a confirmation of ALS but suggests that the invasive assay may not yet have sufficient sensitivity to identify disease onset in all patients.

Direct imaging of the spine with a positron emission tomography (PET) and single photon emission computed tomography (SPECT), and also magnetic resonance imaging (MRI), magnetic particle imaging (MPI) and optical imaging have the potential as a diagnostic tool to detect upper motor neuron (UMN) pathology in ALS patients without UMN symptoms, as a prognostic tracer that might help stratify patients for clinical trials, and as a pharmacodynamic tracer that measures the biological effect of investigational drugs in the brain and spinal cord.

PET is a powerful imaging technique that can follow the distribution of picomolar concentrations of radiopharmaceuticals. PET imaging is non-invasive, functional and quantitative and has proven to be a highly valuable technology platform for the study of other neurodegenerative diseases. It also represents a powerful technology to tackle proof-of-efficacy studies for drug development. The availability of sensitive PET imaging scanners for pre-clinical research and the introduction of PET-CT for preclinical and clinical use could be used for an early and certain ALS diagnosis if a specific radiotracer was developed. Similarly, SPECT; MRI, MPI and optical imaging including fluorescence imaging, or combinations thereof and/or with PET (e.g., PET/CT, PET/MRI, PET/MPI) are used to selectively visualize molecular targets for which a specific tracer could be used, e.g., for detection of misfolded SOD1 and diagnosis of ALS and/or monitoring of the disease progression/treatment.

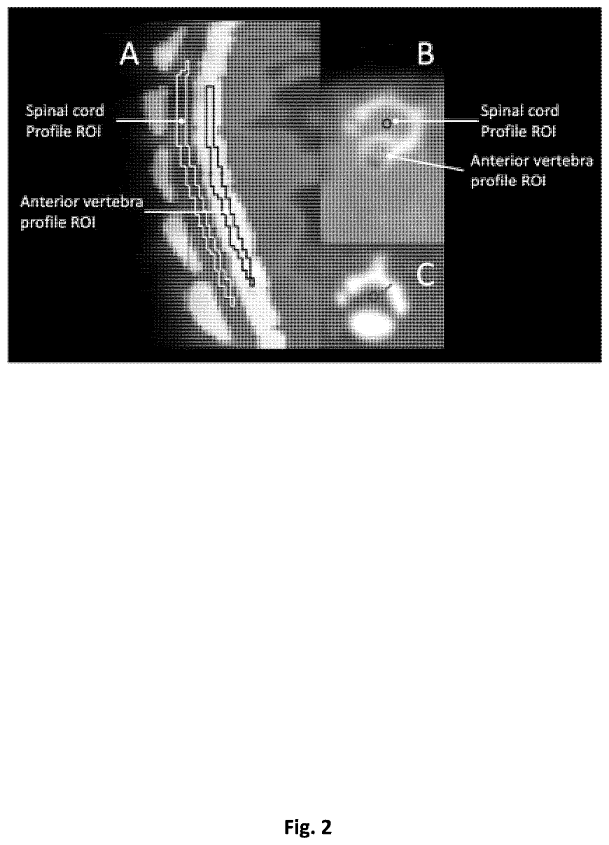

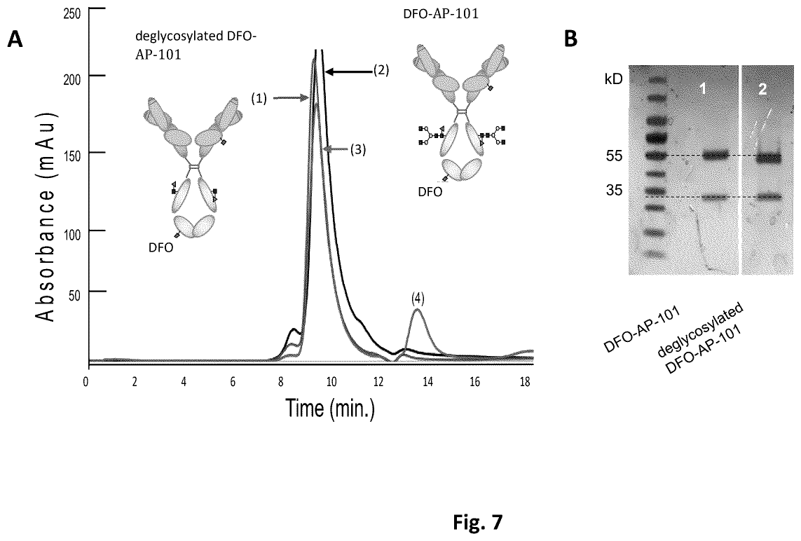

For investigating this approach, a labeled-antibody conjugate, DFO-AP-101 was prepared by conjugating AP-101 antibody with p-SCN-Bn desferoxamine (SCN-Bn-DFO) chelator randomly on lysine residues and the Degly-DFO-AP-101 (deglycosylated antibody-chelator) was prepared via an enzymatic treatment of DFO-AP-101 with an endoglycosidase from Streptococcus pyogenes that specifically hydrolyzes glycans at the Fc glycosylation site of IgG (see Example 1 and corresponding Preparation sections in Materials and Methods below). Both antibodies were analyzed via SDS-PAGE (see FIGS. 7 and SEC-HPLC prior labeling with ⁸⁹Zr, which has been selected as an exemplary radionuclide in view of the intended use of PET for immunoimaging. The specific activity and the number of DFO chelators per antibody were determined to fully characterize the PET tracers (see Example 1 and Table 1). An automated process for the production of ⁸⁹Zr-DFO-AP-101 conjugate was developed using a cassette-based module (see Example 1 and corresponding Preparation sections in Materials and Methods below). Stability studies were performed to establish the formulation and the shelf-life of the radiotracers (see Example 2 and section Stability studies in Materials and Methods). A longitudinal imaging study was performed to identify the optimal mice age and time post administration of ⁸⁹Zr-DFO-AP-101 for the detection misfolded SOD1 aggregation in symptomatic and pre-symptomatic transgenic (Tg, [B6.Cg-Tg(SOD1*G93A)1Gur/J)]) mice expressing misfolded SOD1, leading to the formation of m-SOD1 aggregates. A 3D region of interest (ROI) was drawn to include the whole mSOD1 spots and the radiotracer content was expressed in terms of percent of injected dose per gram equivalent (% ID/g). Control imaging experiments were done with ⁸⁹Zr-DFO-AP-101 and Degly-⁸⁹Zr-DFO-AP-101 in Wt mice. The mice were imaged using a small animal PET/CT scanner following caudal vein administration of either ⁸⁹Zr-DFO-AP-101 or Degly-⁸⁹Zr-DFO-AP-101 (see Examples 3-6, and e.g., FIGS. 2, 3, 13, 16). A group of mice receiving ⁸⁹Zr-DFO-AP-101 were co-injected with unlabeled AP-101 to assess target specificity (see FIG. 3-2 and FIGS. 10, 13 and 19). After the last imaging session (Day-10) the mice were euthanized, the tissues were sampled, and their radiotracer content was measured in a gamma counter.

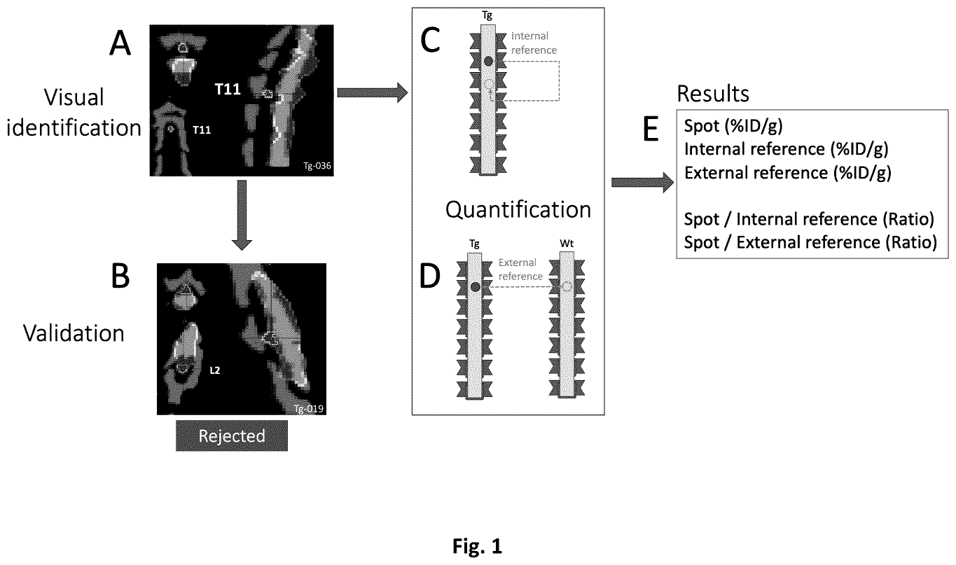

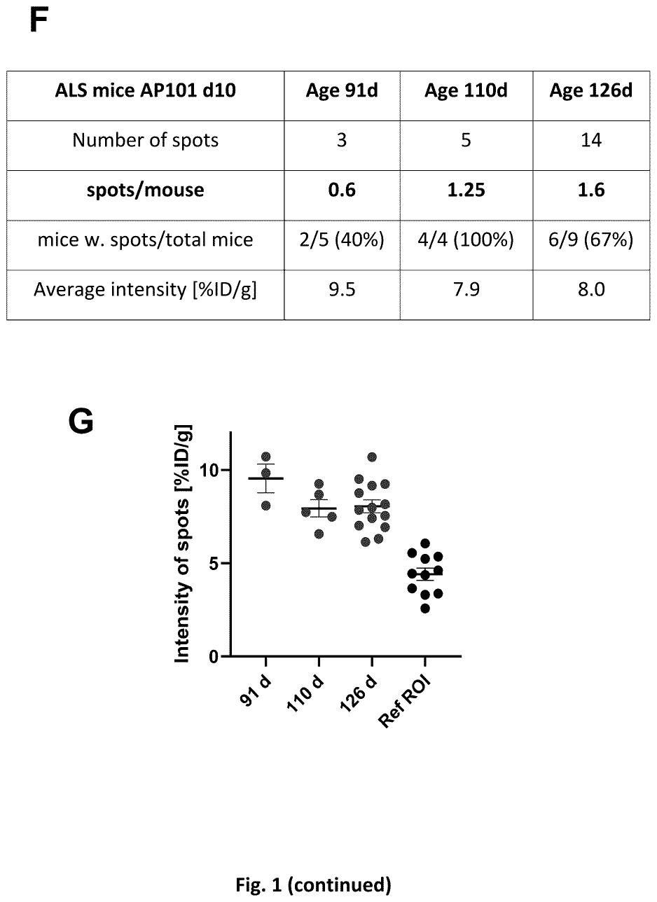

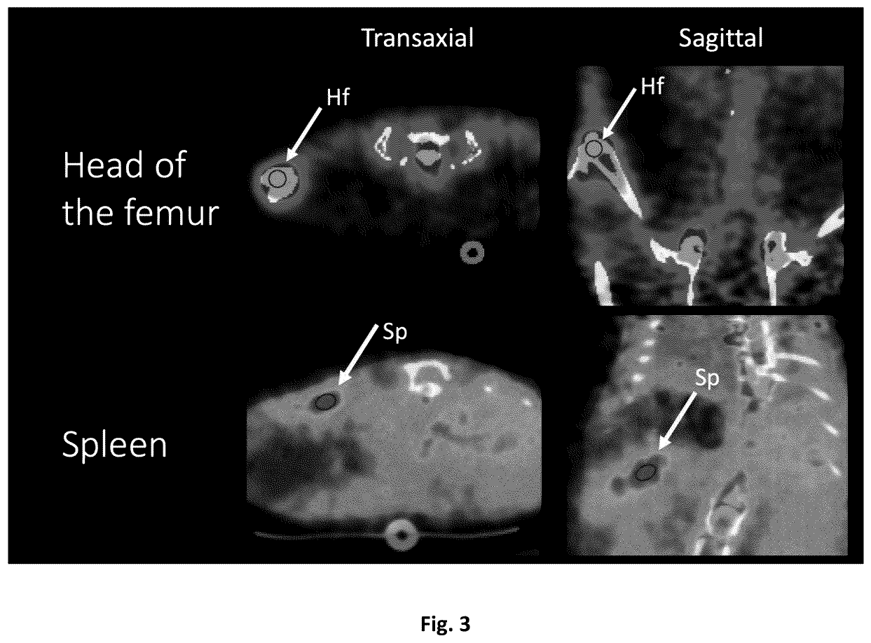

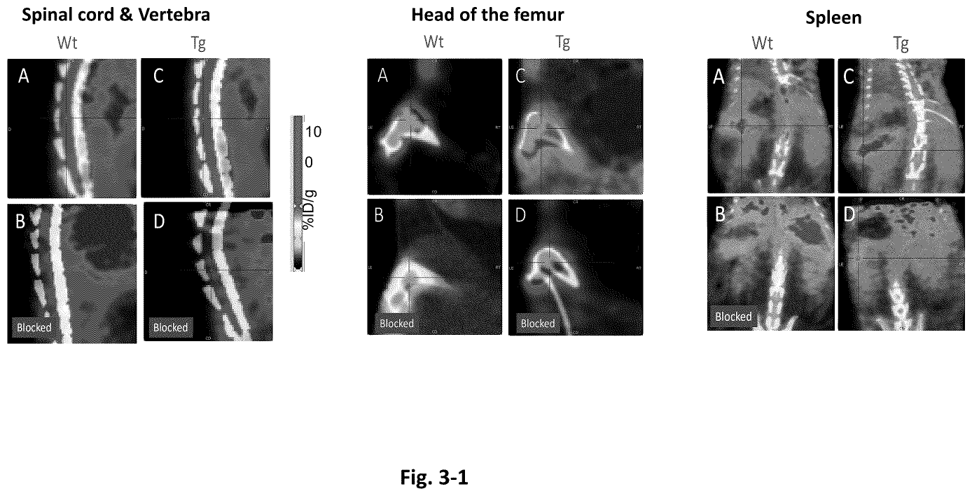

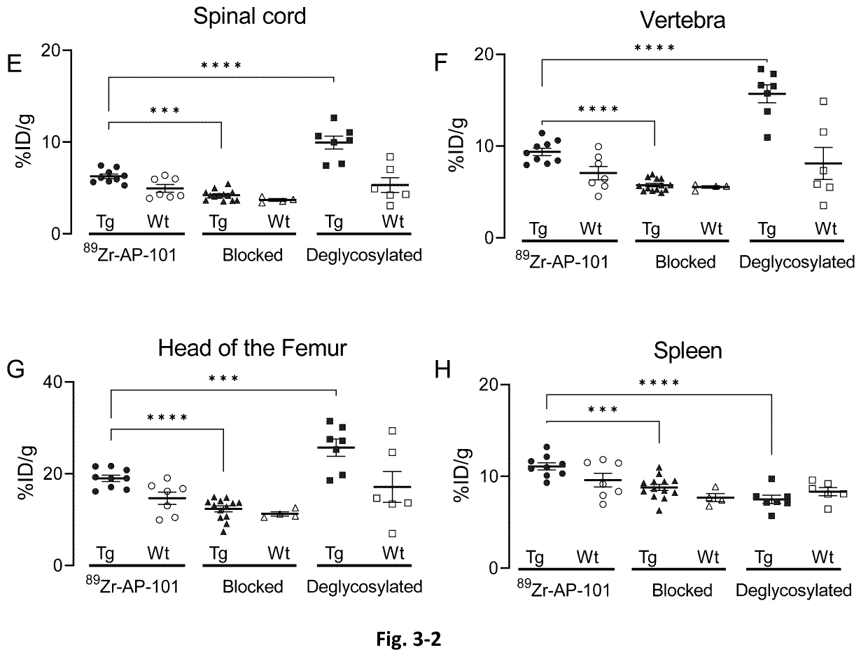

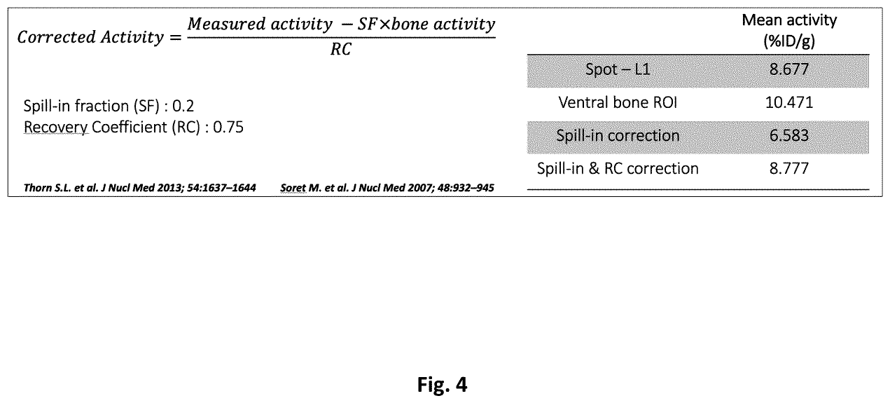

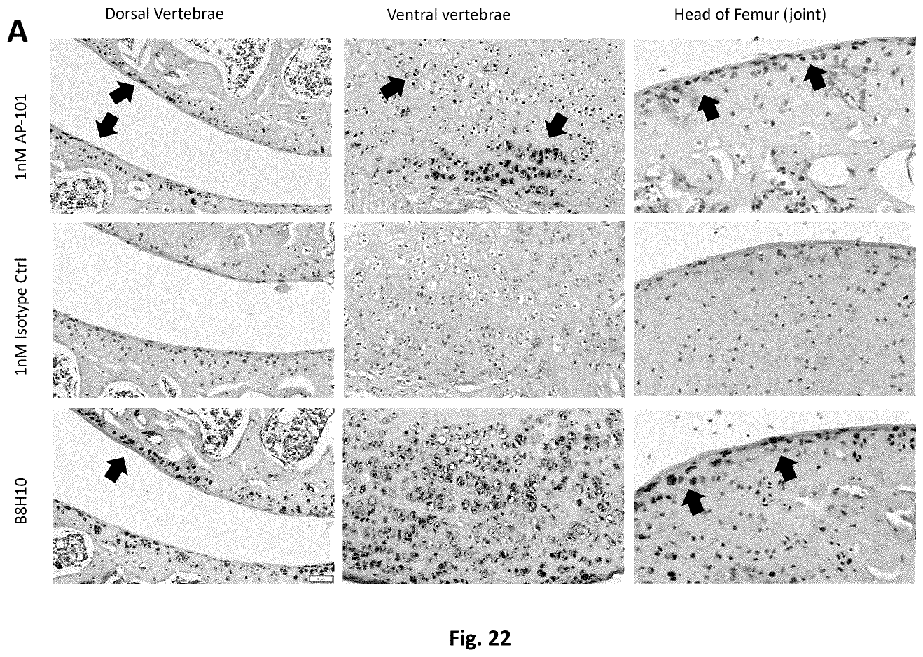

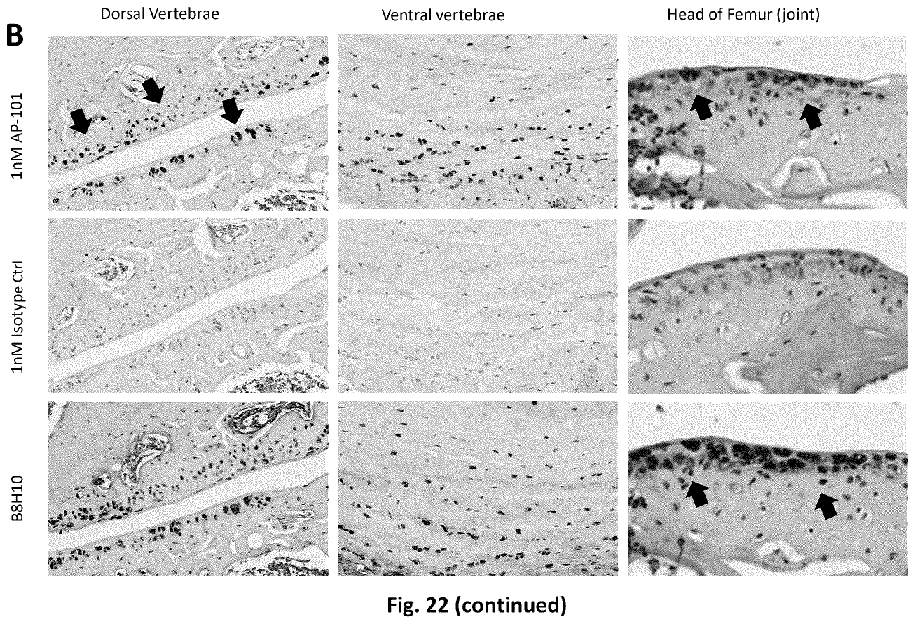

Both radiotracers investigated herein ⁸⁹Zr-DFO-AP-101 and Degly-⁸⁹Zr-DFO-AP-101 were able to engage mSOD1 aggregates in the spinal cord of Tg mice and imaging was optimal in 126-day-old Tg mice and at days 7 and 10 post tracer administration (FIGS. 2-4, 13). The uptake of ⁸⁹Zr-DFO-AP-101 was specific, as it was being blocked when co-injected with AP-101 (FIG. 3-1 and FIGS. 10, 13 and 19). Degly-⁸⁹Zr-DFO-AP-101 showed an improved uptake in mSOD1 aggregate but this tracer was also more concentrated in the anterior vertebra bone of Tg mice causing radiation spilling into the spinal cord resulting in similar aggregate to spinal cord tissue ratios for both radiotracers (FIGS. 3, 4). A low background in spinal cord area of Wt mice was observed for Degly-⁸⁹Zr-DFO-AP-101 resulting in a better contrast between Wt and Tg mice. Spleen and liver uptakes were lower for Degly-⁸⁹Zr-DFO-AP-101 compared to ⁸⁹Zr-DFO-AP-101 (Example 4, FIG. 17 and FIG. 3-2 (continued 2)), but the bone uptake of Tg mice (vertebra and head of the femur) was higher suggesting the presence of mSOD1 in bone and/or bone marrow of Tg mice (Example 5, FIG. 3 and FIG. 22).

Although ⁸⁹Zr-DFO-AP-101 and Degly-⁸⁹Zr-DFO-AP-101 showed similar mSOD1 to spinal cord ratio, Degly-⁸⁹Zr-DFO-AP-101 seems to have the most potential for clinical applications. The rationale for this choice is that the Degly-⁸⁹Zr-DFO-AP-101 was more avidly concentrated in mSOD1 aggregates offering a high contrast between Wt and Tg mice.

In view of the results provided by the experiments described herein and summarized above, the present invention generally relates to a labeled-antibody conjugate for use in a method of diagnosing amyotrophic lateral sclerosis (ALS) and/or monitoring drug therapy and/or progression of ALS in a subject. The method preferably uses in vivo immunoimaging of misfolded SOD1 (mSOD1) in the subject. For this aim the labeled-antibody conjugate preferably comprises an antibody that specifically binds to misfolded and/or aggregated SOD1, and a detection label conjugated to the antibody or antibody-fragment. Preferably, the antibody (with and without label) specifically binds misfolded and/or aggregated mammalian SOD1 such as mouse, rat or human misfolded and/or aggregated SOD1, in particular preferred it binds human misfolded and/or aggregated SOD1.

Preferably the label is either directly or indirectly covalently bound to the antibody. If the label is indirectly conjugated to the antibody, preferably a bifunctional chelator or linker is used, which is then covalently bound to both the label and the antibody.

As mentioned, the labeled-antibody conjugate interchangeably also named as “conjugate” herein (independently of the fact whether the label is directly, or according to the preferred embodiment, indirectly bound to the antibody, e.g., also independently of the fact whether the label is conjugated or complexed) according to the present invention is for use in methods preferably comprising in vivo immunoimaging of mSOD1, by which the conjugate allows, by making mSOD1 visible, to determine whether mSOD1 is present in a subject and if present, in addition to its presence also visualization of mSOD1 distribution in the organs and/or tissues of the subject. Presence of mSOD1, and in particular of mSOD1 comprising aggregates in particular in the spinal cord is, as indicated above, a hallmark for ALS both in patients and in transgenic (Tg) animal models. In this connection, any method known in the art for in vivo immunoimaging can be used. Preferably, however, the particular immunoimaging method is selected from the group comprising or consisting of PET, SPECT, MRI, MPI and optical imaging including fluorescence imaging, and combinations thereof or combinations with CT, such as PET/CT, SPECT/CT and PET/MRI, SPECT/MRI. In particular preferred the imaging or the immunoimaging method respectively comprises or is PET, particularly preferred the imaging or the immunoimaging method respectively comprises or is a combination of PET and CT as exemplarily shown in FIGS. 2, 3 and 21 panel A.

Depending on the imaging method intended to be used for diagnosing ALS or monitoring drug therapy and/or progression of ALS in a subject, the particular label is selected for the labeled-antibody conjugate.

Preferably, the label to be included in the labeled antibody conjugate or labeled antibody fragment conjugate is selected from: Fluorine-18 (F-18), Titanium-45 (Ti-45), Manganese-52 (Mn-52), Iron-52 (Fe-52), Kalium-43 (K-43), Scandium-43 (Sc-43), Scandium-44 (Sc-44), Cobalt-57 (Co-57), Copper-60 (Cu-60), Copper-61 (Cu-61), Copper-62 (Cu-62), Copper-64 (Cu-64), Copper-67 (Cu-67), Gallium-67 (Ga-67), Gallium-68 (Ga-68), Bromine-76 (Br-76), Bromine-77 (Br-77), Krypton-81m (Kr-81m), Rubidium-81 (Rb-81), Yttrium-86 (Y-86), Strontium-87m (Sr-87m), Zirconium-89 (Zr-89 or ⁸⁹Zr), Technetium-99m (Tc-99m), Indium-111 (In-111), Indium-113m (In-113m), Antimony-117 (Sb-117), Iodine-123 (I-123), Iodine-125 (I-125), Caesium-127 (Cs-127), Caesium-129 (Cs-129), Iodine-131 (I-131), Iodine-132 (I-132), Lutetium-177 (Lu-177), Rhenium-186 (Re-186), Quicksilver-197 (Hg-197), Rhenium-188 (Re-188), Lead-203 (Pg-203), Bismuth-206 (Bi-206), Actinium-225 (Ac-225), Radium-225 (Ra-225) and a combination of any two or more thereof.

More preferred, the label is selected from the group comprising or consisting of:
(a) for use in PET from: Fluorine-18 (F-18), Manganese-52 (Mn-52), Copper-64 (Cu-64), Gallium-68 (Ga-68), Antimony-117 (Sb-117), Scandium-43 (Sc-43), Scandium-44 (Sc-44), Titanium-45 (Ti-45), Bromium-76 (Br-76), Rubidium-82 (Rb-82), Yttrium-86 (Y-86), Iodine-124 (I-124) and Zirconium-89 (Zr-89 or ⁸⁹Zr);
(b) for use in SPECT from: Indium-111 (In-111), Technetium-99m (Tc-99m), Iodine-123 Gallium-67 (Ga-67), (I-123), Iodine-131 (I-131), Lutetium-177 and Rhenium-186 (Re-186);
(c) for use in MRI and MPI: magnetic, paramagnetic or superparamagnetic ion complexes or particles, preferably containing Gaddolinum³⁺ (Gd³⁺), Manganese²⁺ (Mn²⁺); Copper²⁺ (Cu²⁺); iron oxide (Fe₃O₄) contrast agents; and
(d) for use in optical imaging including fluorescent imaging from: fluorescein, Cy2, Cy3, Cy3B, Cy3.5, Cy5, Cy5.5, Cy7, Texas Red® (Molecular Probes, Inc., Eugene, OR), AlexaFluor® (Molecular Probes, Inc., Eugene), AlexaFluor 350, AlexaFluor 405, AlexaFluor 430, AlexaFluor 488, AlexaFluor 500, AlexaFluor 532, AlexaFluor 546, AlexaFluor 568, AlexaFluor 594, AlexaFluor 610, AlexaFluor 633, AlexaFluor 647, AlexaFluor 660, AlexaFluor 680, AlexaFluor 700, AlexaFluor 750, BODIPY FL, BODIPY R6G, BODIPY TMR, BOPDIPY 530/550, BODIPY 558/568, BODIPY 564/570, BODIPY 576/589, BODIPY 581/591, BODIPY 630/650, BODIPY 650/665, Rhodamine Green™-X (Molecular Probes, Inc.), Rhodamine Red™-X (Molecular Probes, Inc.), Rhodamine 6G, TMR, TAMRA™ (Applied Biosystems).

Preferably, the labeled antibody-conjugate for use according to the present invention is provided, wherein the detection label is a radionuclide. The label may comprise one or more radionuclides. More preferably, the one or more radionuclide(s) is (are) selected from the radionuclides listed under item (a) or (b) in the preceding paragraph. Mostly preferred, the radionuclide is selected from the radionuclides listed under item (a) in the preceding paragraph.

In particular preferred, the conjugate of a label and an antibody as defined herein, is provided, wherein the radionuclide is Zr-89.

The presence and position of the labeled antibody-conjugate or equivalent binding molecule in the subject is detected in the subject by the selected imaging method preferably in real time. Preferably the imaging method is PET or a combination of PET and CT.

Since in vivo imaging methods, such as PET, require administration of the labeled conjugate to a subject, as a first step, before performing the imaging method, preferably a different screening method is used, which is less invasive. Preferably, an in vitro method is used in this connection, in which marker are detected in a body fluid sample obtained from a patient, which are indicative of ALS. Particularly preferred a mSOD1 screening method is used as described in another application of the present inventors, WO 2021/185961 A1, the content of which is hereby incorporated herein in its entirety by reference.

Accordingly, preferably, before or as confirmation of the immunoimaging method according to the present invention, screening for mSOD1 in a sample of a patient's body fluid is performed (pre-screen). The sample to be analyzed with the assay of the present invention may be any body fluid suspected to contain pathologically mSOD1, for example a blood, CSF, or urine sample. In a preferred embodiment, the sample is whole blood lysate or CSF, preferably CSF.

The screening method applies an ELISA immunoassay comprising contacting the body fluid with two different anti-SOD1 antibodies. In detail, the prescreen is performed by the use of methods as described in Examples 1 to 2 on pages 23 to 25 or as described in Example 3 on pages 26 to 28 of WO 2021/185961 A1, which methods are hereby expressly incorporated herein by reference.

Preferably the detection label is conjugated to the antibody specifically binding to misfolded and/or aggregated SOD1 with a bifunctional chelator or linker, such as a prosthetic group. The bifunctional chelator or prosthetic group can be conjugated in any way resulting in a covalent binding to the antibody. Preferably the chelator or prosthetic group is conjugated randomly on amino acid residues, or to a glycan region of the antibody. Preferably, the amino acid residues on which the chelator or prosthetic group is randomly conjugated are lysine or cysteine residues. Mostly preferred the bifunctional chelator or prosthetic group is conjugated randomly on one or more lysine residues of the antibody.

A detection label is conjugated directly, or indirectly, to an anti-SOD1 antibody to produce a labeled antibody conjugate according to the present invention.

According to embodiments, broadly described, direct conjugation of the detection label to an anti-SOD1 (in particular anti-mSOD1) antibody according to the present disclosure includes reaction of a functional group of the detection label, or a compound containing the detection label, with a corresponding functional group of the antibody, or antibody fragment, such as a terminal amino group, a terminal carboxyl group or a functional group of an amino acid side chain, thereby covalently bonding the detection label with the antibody, or antibody fragment.

According to the present invention, however, indirect conjugation of the detection label to an antibody, or antibody fragment is preferred. Such indirect conjugation includes reaction of a functional group of a linker, prosthetic group, or chelator with a corresponding functional group of the antibody, or -antibody fragment, such as a terminal amino group, a terminal carboxyl group or a functional group of an amino acid side chain, thereby covalently bonding the prosthetic group, or chelator with the antibody, or antibody fragment, and binding of the detection label or a compound comprising the detection label to the linker or chelator.

Functional groups and exemplary conjugation reactions are known in the art as exemplified in G. T. Hermanson, Bioconjugate Techniques, 2^{nd} Edition, Academic Press, 2008.

For indirect conjugation of a detection label to an antibody or antibody fragment, a suitable prosthetic group, or chelator, preferably a bifunctional chelator can be used, wherein the prosthetic group, or chelator is bound to both the antibody, or antibody fragment, and the detection label. Preferably, the prosthetic group, or chelator is bifunctional and therefore functional to bind to both the antibody, or antibody fragment, and the detection label, producing a labeled antibody conjugate.

Prosthetic groups, chelators, and methods of use thereof for conjugation of a detection label to an antibody, or antibody fragment, are well-known in the art, see for example, Shan S. Wong et al., Chemistry of Protein and Nucleic Acid Cross-Linking and Conjugation, Second Edition, CRC Press, 2011. Preferred bifunctional chelators to be used in accordance with the present invention are listed further below.

In embodiments, the labeled-antibody conjugates include at least one detection label conjugated to the antibody, or antibody fragment, wherein the at least one detection label includes a radionuclide. In embodiments, the labeled-antibody conjugates include at least one detection label, is a fluorophore. In embodiments, the at least one detection label is a magnetic/paramagnetic or supermagnetic particle. In embodiments, the labeled-antibody conjugates include at least one detection label, a metal-containing particle. Particular particles include, but are not limited to, carbon-, gold-, or iron-containing nanoparticles.

In embodiments, labeled-antibody conjugates are not limited with respect to the number of labels included. In embodiments, labeled-antibody conjugates include 1, 2, 3, 4, 5, 6, 7, 8, 9, 10 labels, or more. Where multiple labels are included, they may be the same or different.

According to the present invention generally chelators may be used, which have been shown as usable for complexing a radionuclide, a magnetic, paramagnetic or supermagnetic ion complex or particle to an antibody. Preferably the chelator used in accordance with the present invention for complexing labels, preferably radionuclides, to the antibody comprises or is selected from the group consisting of SCN-Bn DFO*, SCN-Bn-HOPO, SCN-Bn-4HMSA, NOTA (2-[4,7-bis(carboxymethyl)-1,4,7-triazonan-1-yl]acetic acid) and its derivatives; DOTA (2-[4,7,10-tris(carboxymethyl)-1,4,7,10-tetrazacyclododec-1-yl]acetic acid) and its derivatives; methylhydroxamates derived from triaza- and tetraazamacrocycles (NOTHA2 and DOTHA2) and its derivatives; 1,4,7-triazacyclononane-1-glutaric acid-4,7-diacetic acid (NODAGA) and its derivatives; diethylenetriaminepentaacetate (DTPA) and its derivatives; 1,4,7,10-tetraazadodecane-1,4,7-triacetate (D03A) and its derivatives; 3,6,9,15-tetraazabicyclo[9.3.1]pentadeca-1 (1 5),11,13-triene-3,6,9-triacetic acid) (PCTA) and its derivatives; 1,4,7,10-tetraazacyclotridecanetetraacetic acid (TRITA) and its derivatives; 1,4,8,11-tetraazacyclotetradecane-1,4,8,11-tetraacetic acid (TETA) and its derivatives; 1,4,7,10-tetraazadodecanetetramethylacetate (DOTMA) and its derivatives; 1,4,7,10-tetraazadodecane-1,4,7-trimethylacetate (D03MA) and its derivatives; N,N′,N″,N′″-tetraphosphonatomethyl-1,4,7,10-tetraazacyclododecane (DOTP) and its derivatives; 1,4,7,10-tetraazacyclododecane-1,4,7,10-tetrakis(methylene methylphosphonic acid) (DOTMP) and its derivatives; 1,4,7,10-tetraazacyclododecane-1,4,7,10-tetrakis(methylene phenylphosphonic acid) (DOTPP) and its derivatives; N,N′-ethylenedi-L-cysteine and its derivatives; N1,N1′-(butane-1,4-diyl)bis(N4-hydroxy-N1-(3-(4-(hydroxy(methyl)amino)-4-oxobutanamido)propyl)-N4-methylsuccinamide) (4HSM) and its derivatives; and p-SCN-Bn desferoxamine (SCN-Bn-DFO; also known asp-SCN-Bn deferoxamine).

In a preferred embodiment, if the antibody is intended to be labeled with Zr-89, generally ⁸⁹Zr-chelators can be used such as desferrichrome (DFC), HMSA, four 1-hydroxypyridin-2-one groups appended to a linear tetraamine (HOPO), L1-4, L5, FSC derivatives, TAFC, FOXE, CP256, YM103, DFO-star (DFO*), oxoDFO* and p-SCN-Bn desferoxamine.

Other chelators can be used in combination with other radiometals: NOTA or its derivatives; methylhydroxamates derived from triaza- and tetraazamacrocycles (NOTHA2 and DOTHA2); 1,4,7-triazacyclononane-1-glutaric acid-4,7-diacetic acid (NODAGA) or its derivatives; diethylenetriaminepentaacetate (DTPA) or its derivatives; 1,4,7,10-tetraazadodecanetetraacetate (DOTA) and its derivatives; 1,4,7,10-tetraazadodecane-1,4,7-triacetate (D03A) and its derivatives; 3,6,9,15-tetraazabicyclo[9.3.1]pentadeca-1(15),11,13-triene-3,6,9-triacetic acid) (PCTA) or its derivatives; 1,4,7,10-tetraazacyclotridecanetetraacetic acid (TRITA) and its derivatives; 1,4,8,11-tetraazacyclotetradecane-1,4,8,11-tetraacetic acid (TETA) and its derivatives; 1,4,7,10-tetraazadodecanetetramethylacetate (DOTMA) and its derivatives; 1,4,7,10-tetraazadodecane-1,4,7-trimethylacetate (D03MA) and its derivatives; N,N′,N″,N′″-tetraphosphonatomethyl-1,4,7,10-tetraazacyclododecane (DOTP) and its derivatives; 1,4,7,10-tetraazacyclododecane-1,4,7,10-tetrakis(methylene methylphosphonic acid) (DOTMP) and its derivatives; 1,4,7,10-tetraazacyclododecane-1,4,7,10-tetrakis(methylene phenylphosphonic acid) (DOTPP) and its derivatives; or N,N′-ethylenedi-L-cysteine or its derivatives.

Mostly preferred, the bifunctional chelator comprises p-SCN-Bn desferoxamine (SCN-Bn-DFO; also known asp-SCN-Bn deferoxamine).

According to the invention, different prosthetic groups can be used for 18F (or F-18)-labeling.

Preferred prosthetic groups for that use comprise N-succinimidyl 4 [18F]-fluorobenzoate ([18F]-SFB), 4-azidophenacyl-[18F]-fluoride ([18F]-APF), and 1-(3-(2-[18F]34luoropyridine-3-yloxy)propyl)pyrrole-2,5-dione ([18F]-FpyMe).

The antibody for use in a method of diagnosing ALS or monitoring drug therapy and/or progression of ALS in a subject as specified herein, can in principle be any anti-SOD1 antibody that selectively binds to misfolded and/or aggregated forms of SOD1, preferably preferentially or exclusively over the physiological form of SOD1.

In principle, the antibody which is part of the labeled antibody conjugate of the present invention may be in any format recognizing misfolded and/or aggregated SOD1 comprising, for example chimeric antibody, single-chain antibody, Fab-fragment, bi-specific antibody (a diabody or minibody), fusion antibody, a unibody or an analog of any one of those. Corresponding methods for producing such variants are known to the person skilled in the art and are described, e.g., in Harlow and Lane “Antibodies, A Laboratory Manual”, CSH Press, Cold Spring Harbor (1988) First edition; Second edition by Edward A. Greenfield, Dana-Farber Cancer Institute © 2014, ISBN 978-1-936113-81-1. For example, Fab and F(ab′)₂ fragments may be produced recombinantly or by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CH1 domain of the heavy chain.

In one embodiment, the antibody for use according to the present invention may thus be provided in a format selected from the group consisting of a single chain Fv fragment (scFv), an F(ab′) fragment, an F(ab) fragment, and an F(ab′)₂ fragment, an Fd, an Fv, a single-chain antibody, a single domain antibody (sdAb, also known as VHH Abs or nanobodies) and a disulfide-linked Fv (sdFv) and/or which is a chimeric murine-human or a humanized antibody. Preferably, the anti-SOD1 antibody employed comprises a human constant domain. The term “equivalent binding molecule” as used herein is intended to refer to the formats indicated as alternatives to an antibody in this and in the preceding paragraph.

The antibody AP-101, which corresponds to antibody NI-204.12G7 as characterized in WO 2012/080518 A1 and in Maier et al., Sci. Transl. Med. 10. (2018) doi: 10.1126/scitranslmed.aah3924 (the disclosure content of which is explicitly incorporated herein by reference in its entirety) is particularly preferred and is a fully human IgG1m3 allotype antibody with selective high affinity binding to misfolded SOD1 protein.

Thus, in one embodiment, the labeled antibody for use in accordance with the present invention, i.e., in particular for use in a diagnosing method as defined above, is an anti-SOD1 antibody recognizing an epitope comprising the amino acid sequence 73-GGPKDEERHVG-83 (SEQ ID NO: 11).

In a particular preferred embodiment the antibody for use in accordance with the present invention is AP-101 and derived from human antibody NI-204.12G7 and characterized by comprising in its variable region, i.e. binding domain the complementarity determining regions (CDRs) of the variable heavy (V_{H}) and variable light (V_{L}) chain having the amino acid sequences depicted in FIG. 1B of WO 2012/080518 A1, and as replicated in Table A, below, or an equivalent antibody wherein one or more of the CDRs may differ in their amino acid sequence from those set forth in FIG. 1B of WO 2012/080518 A1 and in Table A below by one, two, three or even more amino acids in case of CDR2 and CDR3, and wherein the antibody displays substantially the same or identical immunological characteristics of anti-SOD1 antibody NI-204.12G7 illustrated in the Examples of WO 2012/080518 A1. The positions of the CDRs are shown in FIG. 1B and explained in the Figure legend to FIG. 1 in WO 2012/080518 A1, and also shown in Table A below. The corresponding nucleotide sequences are set forth in Table II at page 54 of WO 2012/080518 A1 and listed in Table A below.

Table A: Amino acid and nucleotide sequences of the variable region, i.e., heavy chain and kappa/lambda light chain of human antibodies of the AP-101 antibody. Framework (FR) and complementarity determining regions (CDRs) are indicated with the CDRs being underlined in the amino acid sequences. The heavy chain joining region (JH) and light chain joining region (JK) are indicated as well. Due to the cloning strategy the amino acid sequence at the N-terminus of the heavy chain and light chain may potentially contain primer-induced alterations in FR1, which however do not substantially affect the biological activity of the antibody. In order to provide a consensus human antibody, the nucleotide and amino acid sequences of the original clone were aligned with and tuned in accordance with the pertinent human germ line variable region sequences in the database; see, e.g., Vbase (http://vbase.mrc-cpe.cam.ac.uk/) hosted by the MRC Centre for Protein Engineering (Cambridge, UK). Those amino acids, which are considered to potentially deviate from the consensus germ line sequence due to the PCR primer and thus have been replaced in the amino acid sequence, are indicated in bold.

| | |
|---|---|
| AP-101-V_{H} | GAGGTGCAGCTGGTGGAGTCTGGGGCTGAGGTGAAGAAGCCTGGGGCCTCAGT |
| DNA sequence | GACACTGTCCTGCAAGGCATCTGGATACACCTTCACCGCCTACTATATACACT |
| | GGGTGCGACAGGCCCGAGAACAAGGGCTTGAGTGGATGGGCGTAATCAACCCT |
| | AGTACTGGAACCACATTTTACGCACAGAACTTCCCGGACAGAGTCTCCGTGAC |
| | CAGGGACACGTCCACGAGTACAGTCTTCATGGAGCTGCACAACCTGAAATCTG |
| | AGGACACGGCCGTATATTACTGTGCGAGAGCAATCAGTGAGCATGGTTCAGGG |
| | AGTTATTCACCTTATTACTGGGGCCAGGGCACCCTGGTCACCGTCTCCTCG |
| | SEQ ID NO: 12 |
| AP-101-V_{H} | FR1---------------------------CDR1-FR2----------- |
| amino acid | QVQLVQSGAEVKKPGASVTLSCKASGYTFTAYYIHWVRQAREQGLEWMG |
| sequence | E E |
| | CDR2-------------FR2----------------------------- |
| | VINPSTGTTFYAQNFPDRVSVTRDTSTSTVFMELHNLKSEDTAVYYCAR |
| | CDR3----------JH--------- |
| | AISEHGSGSYSPYYWGQGTLVTVSS SEQ ID NO: 1 (version |
| | with Q and Q) and SEQ ID NO: 2 (version with E and E) |
| AP-101-V_{L} | TCCTATGTGCTGACTCAGCCACCCTCGGTGTCAGTGTCCCTAGGACAGATGGC |
| DNA sequence | CGCGATCACCTGCTCTGGAGAGGCATTGCCAAAAAAGTATGGTTATTGGTACC |
| | AGCAGAAGCCAGGCCAGGTCCCTGTTCTGCTAATTTATAGAGACGTCGAGAGG |
| | CCCTCAGGGGTCCCTGACCGATTCTCTGGCTCCAGCTCAGGGACAATGGTCAC |
| | ATTGACCATCAGTGGAGTCCAGGCAGAGGACGAGGCTGACTATTACTGTCTCT |
| | CAGCAGACAGCAGTGGTACTTGGGTGTTCGGCGGAGGGACCAAGCTGACCGTC |
| | CTA SEQ ID NO: 13 |
| AP-101-V_{L} | FR1--------------------CDR1------FR2------------ |
| amino acid | SYELTQPPSVSVSLGQMAAITCSGEALPKKYGYWYQQKPGQVPVLLIY |
| sequence | V |
| | CDR2---FR3------------------------------CDR3 |
| | RDVERPSGVPDRFSGSSSGTMVTLTISGVQAEDEADYYCLSADSSGTWV |
| | JK-------- |
| | FGGGTKLTVL SEQ ID NO: 6 (version with E), SEQ ID |
| | NO: 7 (version with V at position 3) |

In addition, or alternatively, the framework regions or complete V_{H} and/or V_{L} chain are 8000 identical to the framework regions depicted in Table A above (and FIG. 1B of WO 2012/080518 A1), preferably 85%, 90%, 95%, 96%, 97%, 98%, 99% or 100% identical to the framework regions and V_{H} and/or V_{L} chain, respectively, depicted in Table A above (and FIG. 1B of WO 2012/080518 A1). Furthermore, cloning and expression of antibody NI-204.1B has been performed as described in WO 2012/080518 A1 in the section “Material and methods” at pages 84 to 88 the description of which methods is thus incorporated herein by reference.

In a particular preferred embodiment, the antibody is characterized by the CDRs and/or the V_{H} and/or V_{L} chain depicted in FIG. 1B of WO 2012/080518 A1 and in Table A above.

Thus, the antibody for use as defined herein preferably comprises (besides the detection label conjugated to the antibody as characterized herein):
(i) a variable heavy (VH) chain comprising VH complementary determining regions (CDRs) 1, 2, and 3, and/or a variable light (VL) chain comprising VL CDRs 1, 2, and 3, wherein
   (a) VH-CDR1 comprises the amino acid sequence of SEQ ID NO: 3 or a variant thereof, wherein the variant comprises one or two amino acid substitutions,
   (b) VH-CDR2 comprises the amino acid sequence of SEQ ID NO: 4 or a variant thereof, wherein the variant comprises one or two amino acid substitutions,
   (c) VH-CDR3 comprises the amino acid sequence of SEQ ID NO: 5 or a variant thereof, wherein the variant comprises one or two amino acid substitutions,
   (d) VL-CDR1 comprises the amino acid sequence of SEQ ID NO: 8 or a variant thereof, wherein the variant comprises one or two amino acid substitutions,
   (e) VL-CDR2 comprises the amino acid sequence of SEQ ID NO: 9 or a variant thereof, wherein the variant comprises one or two amino acid substitutions, and
   (f) VL-CDR3 comprises the amino acid sequence of SEQ ID NO: 10 or a variant thereof, wherein the variant comprises one or two amino acid substitutions; and/or
(ii) a VH chain and/or a VL chain, wherein
   (a) the VH chain comprises the amino acid sequence depicted in SEQ ID NO: 1 or 2 or a variant thereof, wherein the variant comprises one or more amino acid substitutions; and
   (b) the VL chain comprises the amino acid sequence depicted in SEQ ID NO: 6 or 7, or a variant thereof, wherein the variant comprises one or more amino acid substitutions;
   preferably wherein the VH and VL chain amino acid sequence is at least 90% identical to SEQ ID NO: 1 or 2 and 6 or 7, respectively.

In one embodiment, the antibody for use as defined herein is provided, wherein the VH chain comprises the amino acid sequence depicted in SEQ ID NO:1 or a variant thereof, wherein the variant comprises one or more amino acid substitutions; and the VL chain comprises the amino acid sequence depicted in SEQ ID NO: 6 or a variant thereof, wherein the variant comprises one or more amino acid substitutions, preferably wherein the VH and VL chain amino acid sequence is at least 90% identical to SEQ ID NO: 1 and 6, respectively.

In one embodiment, the antibody for use as defined herein is provided, wherein the VH chain comprises the amino acid sequence depicted in SEQ ID NO:1 or a variant thereof, wherein the variant comprises one or more amino acid substitutions; and the VL chain comprises the amino acid sequence depicted in SEQ ID NO: 7 or a variant thereof, wherein the variant comprises one or more amino acid substitutions, preferably wherein the VH and VL chain amino acid sequence is at least 90% identical to SEQ ID NO: 1 and 7, respectively.

In one embodiment, the antibody for use as defined herein is provided, wherein the VH chain comprises the amino acid sequence depicted in SEQ ID NO:2 or a variant thereof, wherein the variant comprises one or more amino acid substitutions; and the VL chain comprises the amino acid sequence depicted in SEQ ID NO: 6 or a variant thereof, wherein the variant comprises one or more amino acid substitutions, preferably wherein the VH and VL chain amino acid sequence is at least 90% identical to SEQ ID NO: 2 and 6, respectively.

In one embodiment, the antibody for use as defined herein is provided, wherein the VH chain comprises the amino acid sequence depicted in SEQ ID NO:2 or a variant thereof, wherein the variant comprises one or more amino acid substitutions; and the VL chain comprises the amino acid sequence depicted in SEQ ID NO: 7 or a variant thereof, wherein the variant comprises one or more amino acid substitutions, preferably wherein the VH and VL chain amino acid sequence is at least 90% identical to SEQ ID NO: 2 and 7, respectively.

Accordingly, most preferably the anti-SOD1 antibody for use in a method of diagnosing amyotrophic lateral sclerosis (ALS) or monitoring drug therapy and/or progression of ALS in a subject of the present invention is AP-101 or an equivalent binding molecule or an mSOD1-binding fragment derived from human antibody NI-204.12G7 as characterized in WO 2012/080518 A1 and Maier et al., Sci. Transl. Med. 10. (2018) doi: 10.1126/scitranslmed.aah3924. Therefore, preferably, the anti-SOD1 antibody for use as defined herein is provided, wherein each heavy chain is comprised of 453 amino acid residues having SEQ ID NO: 14, and each light chain is comprised of 213 amino acid residues having SEQ ID: 15.

Furthermore, preferably, the antibody for use as defined herein is produced in CHO-K1 host cells and purified from the cell culture.

The five primary classes of immunoglobulins are IgG, IgM, IgA, IgD and IgE. These are distinguished by the type of heavy chain found in the molecule. IgG molecules have heavy chains known as gamma-chains; IgMs have mu-chains; IgAs have alpha-chains; IgEs have epsilon-chains; and IgDs have delta-chains; see for review, e.g., Schroeder et al., J. Allergy Clin. Immunol. 125 (2010), S41-S52. Furthermore, different subclasses exist, wherein the IgAs are further divided into subclasses IgA1 and IgA2, and wherein IgGs are further divided into subclasses IgG1, IgG2, IgG3, and IgG4. Furthermore, two types of light chain, kappa (κ) and lambda (λ) exist.

In principle, the antibody for use in accordance with the present invention may be of any kind of class and subclass, respectively, and may comprise any kind of light chain, as long as the antibody binds to misfolded and preferably aggregated forms of SOD1, and preferably as long as binding specificity towards SOD1 as indicated in the Examples of WO 2012/080518 A1 for antibody NI-204.12G7 remains unaffected in kind. However, preferably complete IgG antibodies are used, wherein the antibody comprises a constant domain. Accordingly, in one embodiment, the immunoglobulin heavy and/or light chain constant domain present in the antibody as used in accordance with the present invention is of the IgG type, the IgM type, the IgA type, the IgD type or the IgE type, preferably of the IgG type. In one embodiment, the immunoglobulin heavy and/or light chain constant domain present in the antibody as used in accordance with the present invention is of the IgA1, IgA1, IgG1, IgG2, IgG3, or IgG4 subclass, preferably of the IgG1, IgG2, IgG3, or IgG4 subclass and most preferably of the IgG1 subclass.

The antibody for use as defined herein preferably comprises a human Ig constant region. Furthermore, preferably the antibody as defined herein is a human IgG antibody, more preferred a human IgG1 and mostly preferred a human IgG1m3 allotype antibody. Preferably, the antibody for use as defined herein comprises a kappa (κ) or lambda (λ) light chain.

Recombinant expression of complete human IgG1 antibodies with a human or mouse constant domain can be performed substantially as described in the Examples of WO 2012/080518 A1. Preferably, the antibody is a monoclonal antibody or derived from a monoclonal antibody.

There are not only the above-mentioned four subclasses of IgGs but human heavy and light chain genes also exhibit extensive structural polymorphism(s) and, being closely linked, are inherited as a haplotype. Allotypic variants can be immunogenic and provoke antibody responses as a result of allo-immunization. Thus, switching the allotype can be of particular interest to provide non-immunogenic antibody. So far, extensive allotypes (polymorphisms) are known, but focus is put on the serologically defined allotypes. Allotypes of IgG proteins are defined by the expression of unique epitope(s) recognized by unique serologic reagent(s). Allotypes expressed on the constant region of IgG heavy chain are designated as Gm (Genetic marker) together with the subclass, e.g., G1m, and the allotype number (or letter), e.g., G1 ml [or G1m(a)], G3m5 [or G3m(b1)]. Human immunoglobulin allotypes are listed in Table 1 of Jefferis and Lefrance, mAbs 1 (2009), 1-7 and in FIG. 1A of Irani et al., Molecular Immunology 67 (2015), 171-182, which content is herein incorporated by reference. Accordingly, in one embodiment, the antibody for use in accordance with the present invention is of any one of the following allotypes, but not limited thereto: G1 ml, G1m2, G1m3, G1m17, G2m23, G3m21, G3m28, G3m11, G3m5, G3m13, G3m14, G3m10, G3m15, G3m16, G3m6, G3m24, G3m26, G3m27, A2 ml, A2m2, A2m3, Em1, Km1, Km2, and Km3, but preferably of G1m2, G1m3, or G1m17, and most preferably of G1m3.

As explained above, antibody AP-101 is a fully human IgG1m3 allotype antibody and composed of two identical heavy chains of the IgG1 subclass and the IgG1m3 allotype. In addition, as mentioned above, original human antibody NI-204.12G7 is of the L-lambda type and thus, AP-101 is composed of two identical light chains of the lambda subclass. The sequences of the variable heavy (VH) and variable light (VL) chains of AP-101 are set forth in SEQ ID NOs 1 and 2 and 6 and 7, respectively, and the sequences of the corresponding human constant regions are known in the art. For example, each isotype like the IgG1m3 isotype has a unique amino acid sequence of the constant regions of their heavy chains; see Jefferis and Lefrance (2009), supra. Thus, in one embodiment, the antibody as used in accordance with the present invention is characterized by two heavy chains, wherein each heavy chain comprises an amino acid sequence set forth in SEQ ID NO: 14, and by two light chains, wherein each light chain comprises an amino acid sequence set forth in SEQ ID NO: 15. In this preferred embodiment, each heavy chain is comprised of 453 amino-acid residues, and each light chain consists of 213 amino acid residues. The four chains are preferably stabilized by twelve intra-chain disulfide bonds and four inter-chain disulfide bonds. Each heavy chain preferably contains a single N-linked glycosylation site at Asn303. The N-linked glycosylation structure is preferably predominantly a fucosylated, complex biantennary glycan with 0 galactose residues (GOF) or with 1 galactose residue (G1F).

In addition, but to a minor extent and preferably in negligible amounts, some antibody species for use as described herein may have undergone other post-translational modifications (PTMs) such as partial cleavage, oxidation, deamidation, succinimide or pyroglutamate formation and isomerization. The PTMs identified to be present in AP-101 are shown in Example 7 at pages 104 and 105 and in particular in Table 18 at page 105 of WO 2024/042250 A1, the disclosure of which is incorporated herein by reference.

In one preferred embodiment, the antibody for use in accordance with the present invention has a heavy chain that does not comprise a C-terminal lysine. For example, in such embodiment, the C-terminal lysine included in SEQ ID NO: 14 is absent.

In addition, or alternatively, the antibody for use in accordance with the present invention has a heavy chain, in which the glutamine at the N-terminal is substituted with pyroglutamate. This pyroglutamate formation is also referred to as N-terminal cyclization.

In addition, or alternatively, the antibody for use in accordance with the present invention has a heavy chain which is N-glycosylated, preferably wherein the N-linked glycosylation site is Asn303.

Most preferably, the antibody for use in accordance with the present invention has a heavy chain that does not comprise a C-terminal lysine, i.e., which C-terminal lysine has undergone C-terminal lysine clipping, in which the glutamine at the N-terminal is substituted with pyroglutamate, i.e., which has undergone N-terminal glutaminyl cyclization, and which is N-glycosylated.

The amino acid sequences of the heavy and light chains are shown below:

| | |
|---|---|
| 1 | QVQLVQSGAE VKKPGASVTL SCKASGYTFT AYYIHWVRQA REQGLEWMGV |
| 51 | INPSTGTTFY AQNFPDRVSV TRDTSTSTVF MELHNLKSED TAVYYCARAI |
| 101 | SEHGSGSYSP YYWGQGTLVT VSSASTKGPS VFPLAPSSKS TSGGTAALGC |
| 151 | LVKDYFPEPV TVSWNSGALT SGVHTFPAVL QSSGLYSLSS VVTVPSSSLG |
| 201 | TQTYICNVNH KPSNTKVDKR VEPKSCDKTH TCPPCPAPEL LGGPSVFLFP |
| 251 | PKPKDTLMIS RTPEVTCVVV DVSHEDPEVK FNWYVDGVEV HNAKTKPREE |
| 301 | QYNSTYRVVS VLTVLHQDWL NGKEYKCKVS NKALPAPIEK TISKAKGQPR |
| 351 | EPQVYTLPPS REEMTKNQVS LTCLVKGFYP SDIAVEWESN GQPENNYKTT |
| 401 | PPVLDSDGSF FLYSKLTVDK SRWQQGNVFS CSVMHEALHN HYTOKSLSLS |
| 451 | PGK |

(SEQ ID NO: 14, AP-101-HC, heavy chain amino acid sequence, wherein the amino acids of the constant region are underlined, and wherein the C-terminal lysine (K) is optional and/or the N-terminal glutamine (Q) undergoes intramolecular cyclization, resulting in the formation of pyroglutamic acid)

| | |
|---|---|
| 1 | SYELTQPPSV SVSLGQMAAI TCSGEALPKK YGYWYQQKPG QVPVLLIYRD |
| 51 | VERPSGVPDR FSGSSSGTMV TLTISGVQAE DEADYYCLSA DSSGTWVFGG |
| 101 | GTKLTVLGQP KAAPSVTLFP PSSEELQANK ATLVCLISDF YPGAVTVAWK |
| 151 | ADSSPVKAGV ETTTPSKQSN NKYAASSYLS LTPEQWKSHR SYSCQVTHEG |
| 201 | STVEKTVAPT ECS |

(SEQ ID NO: 15, AP-101-LC, light chain amino acid sequence, wherein the amino acids of the constant region are underlined)

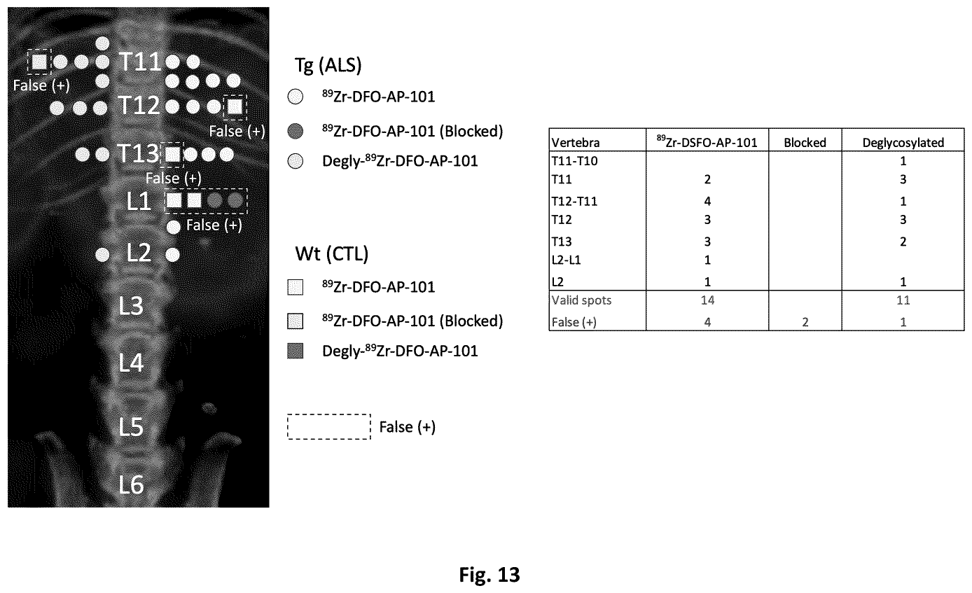

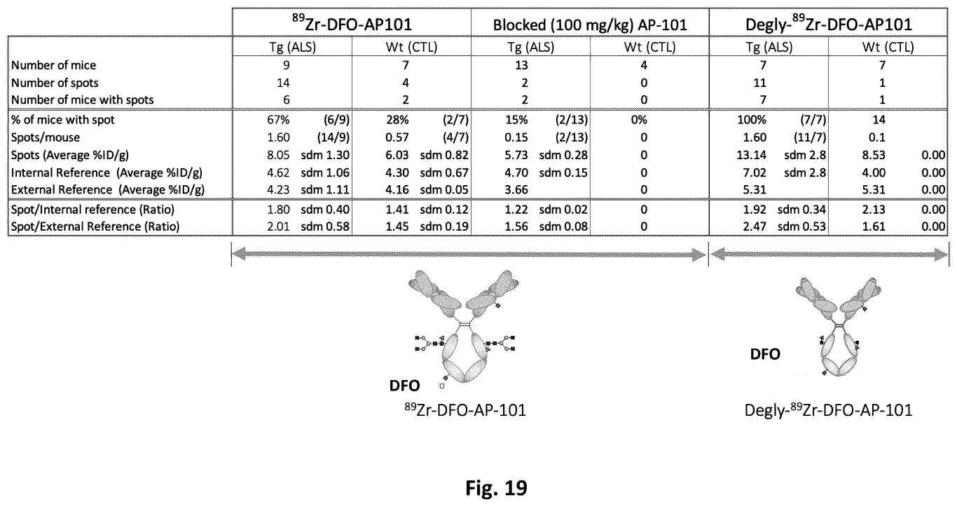

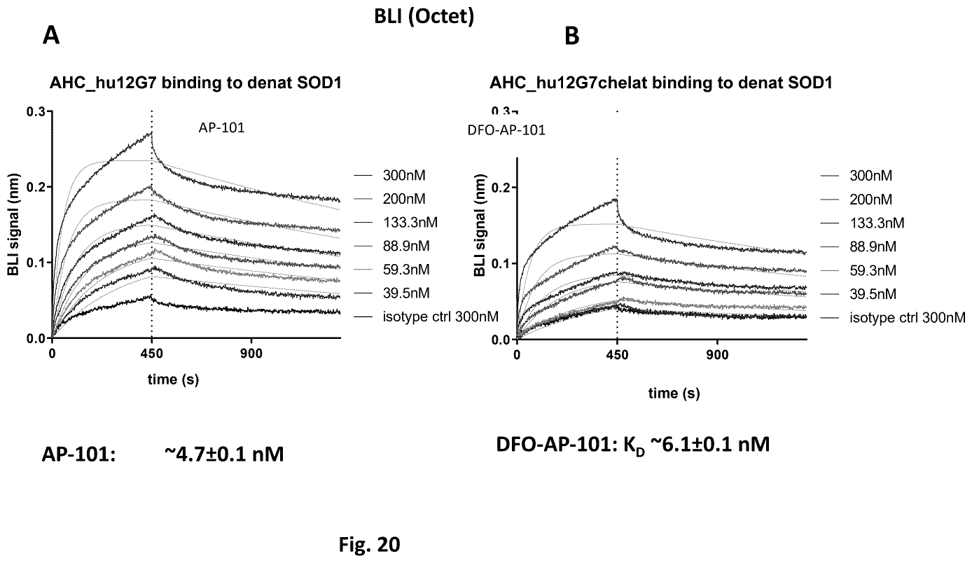

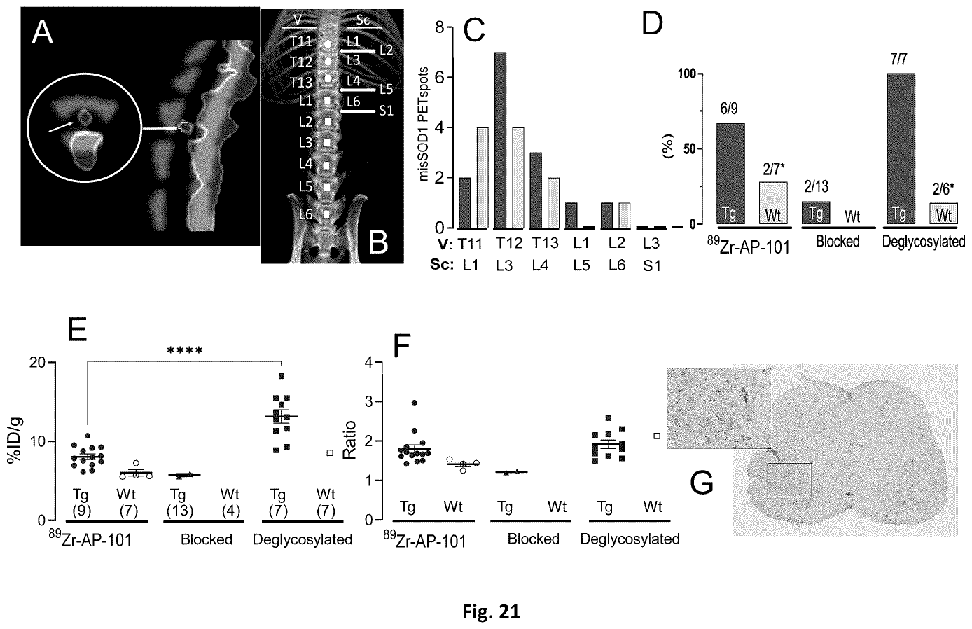

Glycosylation, i.e., attachment of glycans, is a common post-translational modification that occurs during the production of antibodies (Wang et al. (2020)). As shown in the experiments described in the Examples below, deglycosylation increased the efficacy of the radiotracer to detect mSOD1 aggregates from 67% to 100% (Example 3, FIG. 14). However, both radiotracers (i.e., also the glycosylated) were found to be able to engage spinal cord mSOD1 aggregates, the highest uptake and number of detected mSOD1 aggregates being obtained with Degly-⁸⁹Zr-DFO-AP-101 (FIG. 13, 14). Deglycosylation significantly increased the uptake of the radiotracer in vertebra bone and the head of the femur (Example 5, FIG. 3-2 panel G) suggesting the presence of mSOD1 in bone or bone marrow and a specific uptake in these tissues. Moreover, lower spinal cord and vertebra background was seen in Wt mice that received Degly-⁸⁹Zr-DFO-AP-101 resulting in better contrast between Wt and Tg mice with this ⁸⁹Zr-based AP-101 radiotracer (Example 4, FIG. 17 and FIG. 3-2). For these reasons, both radiotracers are suitable, however, Degly-⁸⁹Zr-DFO-AP-101 seems to be the most suitable radiotracer for clinical application. Therefore, the antibody for use as specified herein is provided in two forms, in the glycosylated or in the deglycosylated form (FIG. 19 (scheme)).

Preferably, the antibody for use as defined herein is glycosylated. In another preferred embodiment, the antibody for use as defined herein is deglycosylated. Preferably, by the deglycosylation the removal of N-glycans were removed from the antibody (schematically shown in FIG. 19). Deglycosylation can be obtained by different methods according to the invention. Fc-glycans can be deglycosylated, e.g., by an enzymatic treatment with an endoglycosidase acting on complex type N-glycans or the antibody can be produced in a bacterial host such as E. coli, wherein no glycosylation has occurred.

As could be shown herein, the labeled-antibody-conjugates or labeled antibody fragments for use as defined herein allow detection of mSOD1 and mSOD1-aggregates by usual methods of immunoimaging in vivo (Examples 3-6 and, e.g., FIGS. 2, 3, 16). Therefore, preferably the labeled antibody or antibody fragment for use as specified herein is provided, wherein the imaging comprises PET imaging, SPECT imaging, CT imaging MRI, MPI, optical imaging, including fluorescence imaging, or PET/CT and SPECT/CT.

Early detection and quantification of mSOD1 and mSOD1-aggregates in a subject, due to the high sensitivity of immunoimaging methods such as PET, allows an early diagnosis of ALS, as well as tight monitoring of the progress of the disease and/or of the efficacy of a potential treatment. In the experiments described in the Examples below, preparation of a labeled antibody conjugate has been described and said conjugate validated as a sensitive radiotracer for immunoimaging of misfolded SOD1 (mSOD1). By the use of the conjugate in a method of diagnosis, ALS can be early diagnosed in a subject and a treatment started earlier than by a symptom-based diagnosis. In view of the data provided herein, the conjugate for use as defined herein allows a detailed monitoring of the disease state by visualizing mSOD1 and aggregates thereof even in the spinal motor neurons in vivo (see, e.g., FIGS. 3, 13 and 16), allowing thereby monitoring drug therapy and/or progression of ALS in the subject.

Therefore, the present invention also provides a drug for use in the treatment of ALS in patient who has been diagnosed for suffering or developing ALS and/or is monitored for efficacy of drug therapy and/or disease progression of ALS by a method as defined herein. Preferably the drug is selected hereby from the group consisting of Riluzole (Rilutek®), Edaravone (Radicava®), Tofersen (BIIB067), dextromethorphan HBr and quinidine sulfate (Nuedexta®), AP-101, Reldesemtiv (CY5031), Arimoclomol, Levosimendan, Fasudil, pyrimethamine, (Daraprim™), rapamycin, baclofen (Gablofen®, Kemstro®, Lioresal®), diazepam (Diastat®, Valium®), amitriptyline (Elavil®), trihexyphenidyl, Scopoderm® (scopolamine patch), glycopyrrolate (Robinul®), Ravulizumab (also known as BNJ441, ALXN1210, or Ultomiris®) and Eculizumab (also known as Soliris®).

As described in the examples (see Example 1), due to the experiments performed in connection with the present invention, a labeled antibody conjugate was produced and provided as defined herein when describing the conjugate envisaged for its intended use in a method of diagnosing amyotrophic lateral sclerosis (ALS) or monitoring drug therapy and/or progression of ALS in a subject, wherein the method comprises in vivo immunoimaging in a subject and the labeled-antibody conjugate comprises an antibody that specifically binds to misfolded SOD1 and a detection label conjugated to the antibody.

Therefore, in a further aspect, the present invention also provides a labeled antibody conjugate in all its preferred forms as defined hereinbefore.

Preferably, this labeled antibody conjugate or labeled antibody-fragment conjugate comprises:
(i) a variable heavy (VH) chain comprising VH complementary determining regions (CDRs) 1, 2, and 3, and/or a variable light (VL) chain comprising VL CDRs 1, 2, and 3, wherein
(a) VH-CDR1 comprises the amino acid sequence of SEQ ID NO: 3 or a variant thereof, wherein the variant comprises one or two amino acid substitutions,
(b) VH-CDR2 comprises the amino acid sequence of SEQ ID NO: 4 or a variant thereof, wherein the variant comprises one or two amino acid substitutions,
(c) VH-CDR3 comprises the amino acid sequence of SEQ ID NO: 5 or a variant thereof, wherein the variant comprises one or two amino acid substitutions,
(d) VL-CDR1 comprises the amino acid sequence of SEQ ID NO: 8 or a variant thereof, wherein the variant comprises one or two amino acid substitutions,
(e) VL-CDR2 comprises the amino acid sequence of SEQ ID NO: 9 or a variant thereof, wherein the variant comprises one or two amino acid substitutions, and
(f) VL-CDR3 comprises the amino acid sequence of SEQ ID NO: 10 or a variant thereof, wherein the variant comprises one or two amino acid substitutions; and/or
(ii) a VH chain and/or a VL chain, wherein
(a) the VH chain comprises the amino acid sequence depicted in SEQ ID NO: 1 or 2 or a variant thereof, wherein the variant comprises one or more amino acid substitutions; and
(b) the VL chain comprises the amino acid sequence depicted in SEQ ID NO: 6 or 7, or a variant thereof, wherein the variant comprises one or more amino acid substitutions;
   preferably wherein the VH and VL chain amino acid sequence is at least 90% identical to SEQ ID NO: 1 or 2 and 6 or 7, respectively.

More preferred, the labeled antibody conjugate or labeled antibody-fragment conjugate further comprises p-SCN-Bn desferoxamine (SCN-Bn-DFO) as the bifunctional chelator, and ⁸⁹Zr as the radionuclide tracer.

Particularly preferred, the labeled antibody conjugate is ⁸⁹Zr-DFO-AP-101 and comprises AP-101 as the antibody that specifically binds to misfolded SOD1, p-SCN-Bn desferoxamine (SCN-Bn-DFO) as the bifunctional chelator/linker and Zr-89 as the radionuclide.

As discussed above, antibodies usually occur in glycosylated forms. In a preferred embodiment, the antibody for uses as disclosed herein is glycosylated thus. However, as shown in the Examples and also discussed above, a deglycosylated tracer may be advantageous in immunoimaging due to its increased efficacy to detect mSOD1 aggregates also in tissues in which the glycosylated labeled antibody conjugate does not provide a signal, or only a weak signal. Therefore, in another preferred embodiment, the labeled antibody conjugate is deglycosylated. More preferred the labeled antibody conjugate in this embodiment is deglycosylated ⁸⁹Zr-DFO-AP-101 (Degly-⁸⁹Zr-DFO-AP-101). The exemplary preparation of such a conjugate is described in the Examples, Materials and Methods, subsection “Preparation of Degly-DFO-AP-101” below.

In a further aspect, the present invention also provides an antibody that specifically binds to misfolded SOD1, which antibody comprises a bifunctional chelator, which chelator preferably is p-SCN-Bn desferoxamine (SCN-Bn-DFO).

The antibody specifically binding to misfolded SOD1 preferably comprises:
(i) a variable heavy (VH) chain comprising VH complementary determining regions (CDRs) 1, 2, and 3, and/or a variable light (VL) chain comprising VL CDRs 1, 2, and 3, wherein
   (a) VH-CDR1 comprises the amino acid sequence of SEQ ID NO: 3 or a variant thereof, wherein the variant comprises one or two amino acid substitutions,
   (b) VH-CDR2 comprises the amino acid sequence of SEQ ID NO: 4 or a variant thereof, wherein the variant comprises one or two amino acid substitutions,
   (c) VH-CDR3 comprises the amino acid sequence of SEQ ID NO: 5 or a variant thereof, wherein the variant comprises one or two amino acid substitutions,
   (d) VL-CDR1 comprises the amino acid sequence of SEQ ID NO: 8 or a variant thereof, wherein the variant comprises one or two amino acid substitutions,
   (e) VL-CDR2 comprises the amino acid sequence of SEQ ID NO: 9 or a variant thereof, wherein the variant comprises one or two amino acid substitutions, and
   (f) VL-CDR3 comprises the amino acid sequence of SEQ ID NO: 10 or a variant thereof, wherein the variant comprises one or two amino acid substitutions; and/or
(ii) a VH chain and/or a VL chain, wherein
   (a) the VH chain comprises the amino acid sequence depicted in SEQ ID NO: 1 or 2 or a variant thereof, wherein the variant comprises one or more amino acid substitutions; and
   (b) the VL chain comprises the amino acid sequence depicted in SEQ ID NO: 6 or 7, or a variant thereof, wherein the variant comprises one or more amino acid substitutions;
      preferably wherein the VH and VL chain amino acid sequence is at least 90% identical to SEQ ID NO: 1 or 2 and 6 or 7, respectively.

As illustrated in the Examples and described herein, most preferably, the antibody specifically binding to misfolded and/or aggregated SOD1 is AP-101, i.e., an antibody which comprises
(i) a variable heavy (VH) chain comprising VH complementary determining regions (CDRs) 1, 2, and 3, and a variable light (VL) chain comprising VL CDRs 1, 2, and 3, wherein
   (a) VH-CDR1 comprises the amino acid sequence of SEQ ID NO: 3,
   (b) VH-CDR2 comprises the amino acid sequence of SEQ ID NO: 4,
   (c) VH-CDR3 comprises the amino acid sequence of SEQ ID NO: 5,
   (d) VL-CDR1 comprises the amino acid sequence of SEQ ID NO: 8,
   (e) VL-CDR2 comprises the amino acid sequence of SEQ ID NO: 9, and
   (f) VL-CDR3 comprises the amino acid sequence of SEQ ID NO: 10; and
(ii) a VH chain and a VL chain, wherein
   (a) the VH chain comprises the amino acid sequence depicted in SEQ ID NO: 1 or 2; and
   (b) the VL chain comprises the amino acid sequence depicted in SEQ ID NO: 6 or 7.

The sequences of the constant regions of the heavy chains are known in the art. For example, each isotype and like the IgGlm3 isotype has a unique amino acid sequence of the constant regions of their heavy chains; see Jefferis and Lefrance (2009), supra. Thus, in one embodiment, the antibody as used in accordance with the present invention is characterized by two heavy chains, wherein each heavy chain comprises an amino acid sequence set forth in SEQ ID NO: 14, and by two light chains, wherein each light chain comprises an amino acid sequence set forth in SEQ ID NO: 15. In this preferred embodiment, each heavy chain is comprised of 453 amino-acid residues, and each light chain consists of 213 amino acid residues

Preferably, the antibody (and consequently also the labeled antibody-conjugates as described herein) specifically binds misfolded and/or aggregated mammalian SOD1 such as mouse, rat or human misfolded SOD1, in particular preferred it binds human misfolded SOD1. As explained in detail herein, the labeled antibody-conjugates of the present invention have been developed to provide new possibilities for imaging of misfolded and/or aggregated mammalian SOD1 in animals, and in particular in humans suffering from diseases associated with the occurrence of such misfolded and/or aggregated SOD1. Therefore, in one embodiment an imaging composition is provided, wherein the composition comprises the labeled-antibody conjugate and further comprises NaCl (to make the solution more physiological) and gentisic acid (to avoid radiolysis) at a pH of 4.5 to 8.0. Preferably this imaging composition is provided in a sealed vial (to keep the composition is a sterile and pyrogen-free). The composition is preferably in a volume of 10 mL, constituted from saline (0.9% NaCl) and 10 mg gentisic acid. The vial preferably contains between about 300 and about 500 MBq. Preferably the amount of labeled-antibody conjugate vs. unlabeled antibody is greater than 97% and/or the labeled-antibody conjugate has an apparent specific activity of about 0.66 GBq/mg, optionally wherein the injected solution can be diluted in NaCl prior injection. The final volume of the imaging composition for administration to the animal or human is preferably based on the HEPES or gentisic acid contents for which the specifications preferably are ≤200 μg/injection and ≤5 mg/injection respectively.

In view of the capability of the labeled antibody-conjugates as described herein to detect misfolded and/or aggregated SOD1 in vivo in a subject as described herein and shown in the Examples and in the Figures, the present invention in a further aspect relates to a diagnostic composition or kit comprising the imaging composition as defined above, or the labeled antibody conjugate or antibody as described herein, optionally further comprising a detection label if the antibody is in the diagnostic composition or kit. Preferably the antibody comprised in the diagnostic composition or kit is AP-101 or deglycosylated AP-101. Preferably the AP-101 antibody comprises a bifunctional chelator, which preferably isp-SCN-Bn desferoxamine (SCN-Bn-DFO).

The detection label comprised in said diagnostic composition or kit is selected as described above for the labeled antibody conjugate, labeled antibody-fragment conjugate. This means, depending on the intended use, preferably the label is selected from the group comprising or consisting of:
(a) for use in PET from: Fluorine-18 (F-18), Manganese-52 (Mn-52), Copper-64 (Cu-64), Gallium-68 (Ga-68), Antimony-117 (Sb-117), Scandium-43 (Sc-43), Scandium-44 (Sc-44), Titanium-45 (Ti-45), Bromium-76 (Br-76), Yttrium-86 (Y-86), Iodine-124 (I-124) and Zirconium-89 (Zr-89 or ⁸⁹Zr);
(b) for use in SPECT from: Indium-111 (In-111), Technetium-99m (Tc-99m), Gallium-67 (Ga-67), Iodine-123 (I-123), Iodine-131 (I-131), Lutetium-177 and Rhenium-186 (Re-186)
(c) for use in MRI and MPI: magnetic, paramagnetic or superparamagnetic ion complexes or particles, preferably containing Gaddolinum³⁺ (Gd³⁺), Manganese²⁺ (Mn²⁺); Copper²⁺ (Cu²⁺); iron oxide (Fe₃O₄) contrast agents; and
(d) for use in optical imaging including fluorescent imaging from: fluorescein, Cy2, Cy3, Cy3B, Cy3.5, Cy5, Cy5.5, Cy7, Texas Red® (Molecular Probes, Inc., Eugene, OR), AlexaFluor® (Molecular Probes, Inc., Eugene), AlexaFluor 350, AlexaFluor 405, AlexaFluor 430, AlexaFluor 488, AlexaFluor 500, AlexaFluor 532, AlexaFluor 546, AlexaFluor 568, AlexaFluor 594, AlexaFluor 610, AlexaFluor 633, AlexaFluor 647, AlexaFluor 660, AlexaFluor 680, AlexaFluor 700, AlexaFluor 750, BODIPY FL, BODIPY R6G, BODIPY TMR, BOPDIPY 530/550, BODIPY 558/568, BODIPY 564/570, BODIPY 576/589, BODIPY 581/591, BODIPY 630/650, BODIPY 650/665, Rhodamine Green™-X (Molecular Probes, Inc.), Rhodamine Red™-X (Molecular Probes, Inc.), Rhodamine 6G, TMR, TAMRA™ (Applied Biosystems).

Preferably the detection label comprised in the diagnostic composition or kit is a radionuclide. More preferred it is a radionuclide selected from the group comprising or consisting of the radionuclides in items (a) to (c) above. Even more preferred, the diagnostic composition or kit is provided, wherein the detection label is a radionuclide selected from the group comprising or consisting of Fluorine-18 (F-18), Copper-64 (Cu-64), Gallium-67 (Ga-67), Gallium-68 (Ga-68), Antimony-117 (Sb-117), Scandium-43 (Sc-43), Scandium-44 (Sc-44), Titanium-45 (Ti-45), Indium-111 (In-111), Lutetium-177 (Lu-177) and Zirconium-89 (Zr-89 or ⁸⁹Zr). In particular preferred the detection label is a radionuclide, wherein the radionuclide is ⁸⁹Zr.

Preferably the labeled antibody conjugate or antibody comprised in the diagnostic composition or kit is ⁸⁹Zr-DFO-AP-101 or deglycosylated ⁸⁹Zr-DFO-AP-101 (Degly-⁸⁹Zr-DFO-AP-101).

The diagnostic composition or kit optionally further comprises instructions for use in a method of diagnosing amyotrophic lateral sclerosis (ALS) or monitoring drug therapy and/or progression of ALS in a subject wherein the method comprises in vivo immunoimaging of in subject and the labeled-antibody conjugate comprises an antibody that specifically binds to misfolded SOD1 and a detection label conjugated to the antibody as defined hereinabove.

In a further interrelated aspect, the present invention also refers to a method of in vivo immunoimaging of superoxide dismutase (SOD1) useful as a marker of amyotrophic lateral sclerosis (ALS) or as a marker for efficacy of drug therapy of ALS and/or progression of ALS in subject, comprising
(a) administering a labeled-antibody conjugate as described herein to a subject; and
(b) detecting the presence of the labeled-antibody conjugate in the subject in vivo by imaging.

Preferably the antibody which is used in this method of in vivo immunoimaging is an antibody which specifically binds to misfolded SOD1 as specified hereinbelow and further preferred, the subject is a human.

The antibody specifically binding to misfolded SOD1 which is administered in said methods of in vivo immunoimaging preferably comprises:
(i) a variable heavy (VH) chain comprising VH complementary determining regions (CDRs) 1, 2, and 3, and/or a variable light (VL) chain comprising VL CDRs 1, 2, and 3, wherein
   (a) VH-CDR1 comprises the amino acid sequence of SEQ ID NO: 3 or a variant thereof, wherein the variant comprises one or two amino acid substitutions,
   (b) VH-CDR2 comprises the amino acid sequence of SEQ ID NO: 4 or a variant thereof, wherein the variant comprises one or two amino acid substitutions,
   (c) VH-CDR3 comprises the amino acid sequence of SEQ ID NO: 5 or a variant thereof, wherein the variant comprises one or two amino acid substitutions,
   (d) VL-CDR1 comprises the amino acid sequence of SEQ ID NO: 8 or a variant thereof, wherein the variant comprises one or two amino acid substitutions,
   (e) VL-CDR2 comprises the amino acid sequence of SEQ ID NO: 9 or a variant thereof, wherein the variant comprises one or two amino acid substitutions, and
   (f) VL-CDR3 comprises the amino acid sequence of SEQ ID NO: 10 or a variant thereof, wherein the variant comprises one or two amino acid substitutions; and/or
(ii) a VH chain and/or a VL chain, wherein
   (a) the VH chain comprises the amino acid sequence depicted in SEQ ID NO: 1 or 2 or a variant thereof, wherein the variant comprises one or more amino acid substitutions; and
   (b) the VL chain comprises the amino acid sequence depicted in SEQ ID NO: 6 or 7, or a variant thereof, wherein the variant comprises one or more amino acid substitutions;
      preferably wherein the VH and VL chain amino acid sequence is at least 90% identical to SEQ ID NO: 1 or 2 and 6 or 7, respectively.

More preferred, the antibody specifically binding to misfolded SOD1 administered in the methods of in vivo immunoimaging is AP-101.

Preferably the antibody which is administered to the subject in the immunoimaging method comprises a bifunctional chelator, which preferably is p-SCN-Bn desferoxamine (SCN-Bn-DFO).

Furthermore, the antibody is preferably labeled by a detection label, wherein depending on the intended use, the detection label is selected from the group comprising or consisting of:
(a) for use in PET from: Fluorine-18 (F-18), Manganese-52 (Mn-52), Copper-64 (Cu-64), Gallium-68 (Ga-68), Antimony-117 (Sb-117), Scandium-43 (Sc-43), Scandium-44 (Sc-44), Titanium-45 (Ti-45), Bromium-76 (Br-76), Yttrium-86 (Y-86), Iodine-124 (I-124) and Zirconium-89 (Zr-89 or ⁸⁹Zr);
(b) for use in SPECT from: Indium-111 (In-111), Technetium-99m (Tc-99m), Gallium-67 (Ga-67), Iodine-123 (I-123), Iodine-131 (I-131), Lutetium-177 and Rhenium-186 (Re-186)
(c) for use in MRI and MPI: magnetic, paramagnetic or superparamagnetic ion complexes or particles, preferably containing Gaddolinum³⁺ (Gd³⁺), Manganese²⁺ (Mn²⁺); Copper²⁺ (Cu²⁺); iron oxide (Fe₃O₄) contrast agents; and
   (d) for use in optical imaging including fluorescent imaging from: fluorescein, Cy2, Cy3, Cy3B, Cy3.5, Cy5, Cy5.5, Cy7, Texas Red® (Molecular Probes, Inc., Eugene, OR), AlexaFluor® (Molecular Probes, Inc., Eugene), AlexaFluor 350, AlexaFluor 405, AlexaFluor 430, AlexaFluor 488, AlexaFluor 500, AlexaFluor 532, AlexaFluor 546, AlexaFluor 568, AlexaFluor 594, AlexaFluor 610, AlexaFluor 633, AlexaFluor 647, AlexaFluor 660, AlexaFluor 680, AlexaFluor 700, AlexaFluor 750, BODIPY FL, BODIPY R6G, BODIPY TMR, BOPDIPY 530/550, BODIPY 558/568, BODIPY 564/570, BODIPY 576/589, BODIPY 581/591, BODIPY 630/650, BODIPY 650/665, Rhodamine Green™-X (Molecular Probes, Inc.), Rhodamine Red™-X (Molecular Probes, Inc.), Rhodamine 6G, TMR, TAMRA™ (Applied Biosystems).

The label is preferably covalently bound to the antibody via the bifunctional chelator or via a prosthetic group, wherein preferably the bifunctional chelator or prosthetic group is conjugated randomly on one or more lysine or cysteine residues of the antibody. More preferred, the bifunctional chelator or prosthetic group is conjugated randomly on oner or more lysine residues.

Preferably the detection label is a radionuclide. More preferred it is a radionuclide selected from the group comprising or consisting of the radionuclides in items (a) to (c) above. Even more preferred, the method of immunoimaging is provided, wherein the detection label is a radionuclide selected from the group comprising or consisting of Fluorine-18 (F-18), Copper-64 (Cu-64), Gallium-67 (Ga-67), Gallium-68 (Ga-68), Antimony-117 (Sb-117), Scandium-43 (Sc-43), Scandium-44 (Sc-44), Titanium-45 (Ti-45), Indium-111 (In-111), Lutetium-177 (Lu-177) and Zirconium-89 (Zr-89 or ⁸⁹Zr). Particularly preferred the detection label is a radionuclide, wherein the radionuclide is ⁸⁹Zr.

Preferably the labeled antibody conjugate or antibody used in the immunoimaging method is ⁸⁹Zr-DFO-AP-101 or deglycosylated ⁸⁹Zr-DFO-AP-101 (Degly-⁸⁹Zr-DFO-AP-101).

Preferably, the step of detecting the presence of the labeled-antibody conjugate in the subject in vivo by imaging comprises PET imaging, SPECT imaging, MRI, MPI, optical imaging including fluorescence imaging, or PET and SPECT, preferably in combination with CT (i.e., PET/CT or SPECT/CT), mostly preferred the imaging comprises PET.

### Methods for Imaging

In one or more embodiments, methods for imaging are disclosed herein. In embodiments, methods for imaging include: (a) administering a labeled-antibody conjugate to a subject, wherein the labeled-antibody conjugate includes: an antibody that specifically recognizes and binds to mSOD1, and at least one detection label conjugated to the antibody, wherein the at least one detection label includes at least a label, preferably a radionuclide, a fluorophore, a magnetic/paramagnetic/supermagnetic particle, or a combination or any two or more thereof; and (b) detecting the presence of the labeled conjugate in the subject in vivo by imaging. In embodiments, the labeled-antibody conjugate is as described herein with regard to antibody conjugates. In further embodiments, the antibody and the at least one detection label are as described herein with regard to antibody conjugates.

Preferably, the methods for imaging include detecting the presence of the labeled conjugate in the subject in vivo by imaging. Preferably, the presence of the conjugate is detected in real time. Preferably, the presence of the labeled-antibody conjugate is detected non-invasively and/or minimally invasively.

According to aspects of the present disclosure, an antibody conjugate is an imaging agent which can be used to visualize mSOD1, such as in diagnostic procedures. Imaging can be performed by many procedures well-known to those having ordinary skill in the art, for example, by PET, SPECT, Cerenkov imaging, photoacoustic imaging, ultrasound imaging, optical coherence tomography, optical imaging, including fluorescence imaging, magnetic resonance imaging, magnetic particle imaging, bioluminescence imaging, or a combination of any two or more thereof.

In embodiments, the methods include administering a labeled-antibody conjugate to a subject.

In embodiments, the methods include administering an effective amount of a labeled-antibody conjugate to a subject. The labeled-antibody conjugate may be administered by any suitable route known to those of ordinary skill in the art. Preferably, administration of the labeled-antibody conjugate is by intravenous, intraarterial, intraperitoneal, intramuscular, subcutaneous, intrapleural, intrathecal, perfusion through a regional catheter, and/or direct intralesional injection. If the labeled-antibody conjugate is administered by injection, the administration may be by continuous infusion, by single bolus, and/or by multiple boluses. In some embodiments, administering the radiolabeled-antibody conjugate is nonimmunogenic to the subject. According to embodiments, administering a labeled conjugate to a subject includes administering a pharmaceutical composition including the labeled-antibody conjugate and a pharmaceutically acceptable carrier.

Preferably, the subject, as regards the diagnostic as the therapeutic aspects described therein, is a mammal. In some embodiments, the subject is a mammal selected from: humans, non-human primates, canines, felines, murines, bovines, equines, caprines, ovines, porcines, and lagomorphs. In a preferred embodiment, the subject is a rodent, including, but not limited to, a rat mouse, or guinea pig. In particular preferred, the subject is a mouse or a human.

In preferred embodiments, the labeled-antibody conjugate administered to a subject includes a radionuclide. Preferably, the labeled-antibody conjugate administered to a subject includes a labeled-antibody conjugate wherein the label is a radionuclide only, i.e. without a fluorophore or magnetic/paramagnetic/supermagnetic particle. In some embodiments, the labeled-antibody conjugate administered to a subject includes a labeled-antibody conjugate wherein the label is a fluorophore only, i.e., without a radionuclide or para-/supermagnetic particle. In some embodiments, the labeled-antibody conjugate administered to a subject includes a labeled-antibody conjugate wherein the label is a para-/supermagnetic particle only, i.e., without a radionuclide or fluorophore.

In embodiments wherein the labeled antibody conjugate includes a fluorophore, the presence of the labeled-antibody conjugate may be detected in vivo by optical imaging. Various in vivo optical imaging techniques are known to those of ordinary skill in the art. (See e.g., Ntziachristos, Annu. Rev. Biomed. Eng. 2006, 8:1-33; Troyan, S. L. et al., Ann. Surg. Oneal. 16, 2943-2952 (2009); Luker, G. D. & Luker, K. E., J. Nucl. Med. 49, 1-4 (2008); Tromberg, B. J. et al., Med. Phys. 35, 2443-2451 (2008), and their potential applicability to imaging-guided diagnostic and surgical methods has been proposed in several preclinical studies; Kirsch, D. G. et al., Nat. Med. 13, 992-997 (2007); von Burstin, J. et al., Int. J. Cancer 123, 2138-2147 (2008); and U.S. Pat. No. 9,409,923).

In embodiments, in vivo optical imaging is selected from confocal microscopy, planar imaging, fluorescence molecular tomography, complete projection tomography, fluorescence tomography direct imaging, two-photon in vivo imaging or a combination of any two or more thereof. In further embodiments, planar imaging is selected from epi-illumination (i.e., photographic) imaging, trans-illumination imaging, tomographic imaging, or a combination of any two or more thereof.

In embodiments wherein the presence of the labeled-antibody conjugate is detected by in vivo optical imaging, the fluorophore may be selected such that it emits fluorescence in the visible or near-infrared region. In illustrative, non-limiting embodiments, fluorophores emitting fluorescence in the visible or near-infrared region may be selected from fluorescein, Cy2, Cy3, Cy3B, Cy3.5, Cy5, Cy5.5, Cy7, Texas Red® (Molecular Probes, Inc., Eugene, OR), AlexaFluor® (Molecular Probes, Inc., Eugene), AlexaFluor 350, AlexaFluor 405, AlexaFluor 430, AlexaFluor 488, AlexaFluor 500, AlexaFluor 532, AlexaFluor 546, AlexaFluor 568, AlexaFluor 594, AlexaFluor 610, AlexaFluor 633, AlexaFluor 647, AlexaFluor 660, AlexaFluor 680, AlexaFluor 700, AlexaFluor 750, BODIPY FL, BODIPY R6G, BODIPY TMR, BOPDIPY 530/550, BODIPY 558/568, BODIPY 564/570, BODIPY 576/589, BODIPY 581/591, BODIPY 630/650, BODIPY 650/665, Rhodamine Green™-X (Molecular Probes, Inc.), Rhodamine Red™-X (Molecular Probes, Inc.), Rhodamine 6G, TMR, TAMRA™ (Applied Biosystems), or a combination of any two or more thereof.

In embodiments the presence of the labeled-antibody conjugate is detected by in vivo photoacoustic imaging. In illustrative, non-limiting embodiments, photoacoustic dyes included in the conjugate may be selected from Methylene blue, Evan's blue, Trypan blue, Patent blue, Indocyanine Green, IRDye800CW, DiR, Cy7, Cy7.5, and porphyrins or a combination of any two or more thereof.

In embodiments wherein the labeled antibody conjugate includes a combination of a radionuclide tracer and a fluorophore, the presence of the labeled-antibody conjugate may be detected in vivo by a combination of PET or SPECT and optical imaging.

In embodiments wherein the labeled antibody conjugate includes a combination of a radionuclide tracer and a fluorophore, the presence of the labeled antibody conjugate may be detected in vivo by a combination of PET or SPECT and photoacoustic imaging.

In embodiments the presence of the labeled-antibody conjugate may be detected in vivo by a combination of any two or more of: PET, SPECT, CT, Cerenkov imaging, photoacoustic imaging, ultrasound imaging, optical coherent tomography, magnetic resonance imaging, magnetic particle imaging, optical imaging, including fluorescence imaging, and/or bioluminescence imaging.

As described in Example 7 and shown in FIG. 22, the Experiments performed in the context of the present application for the first time shown the presence of misfolded and/or aggregated SOD1 in bones and joints of transgenic SOD1 G93A and also G37R mice, which are commonly used in the field as ALS models. Impairment of the bone function and structure, such as affected formation of osteoprogenitors, impaired osteoblast differentiation capacity, increased osteoclast formation and bone resorption leading to a decreased bone stability manifesting in osteopenia and fractures have been observed in ALS patients and ALS model organisms (Caplliure-Llopis at a1. (2020), Zhu et al. (2015), Torres et al., 2021 and Wang et al., (2018)) confirming a relationship between muscles, which are affected in ALS by progressive atrophy and bones. The observations provided herewith, together with the findings described in WO 2012/080518 A1, that application of SOD1 targeting antibodies leads to an amelioration of ALS symptoms, such as slowing down the progressive motoric impairments, reduced loss of body weight in SOD1 transgenic animals and result in a delay of the disease onset, prolongation of survival and improvement of body weight and motor performance, strongly suggest that anti-SOD1 antibodies can be also of use in treatment and/or prevention of bone and/or joints related diseases, e.g., by reducing and potentially preventing or delaying the onset of bone and/or joints deterioration in a disease associated with the presence or accumulation of misfolded and/or aggregated SOD1 in the bones and/or joints, e.g., in ALS.

Accordingly, in a further embodiment, the present invention provides an anti-SOD1 antibody or antigen binding fragment thereof for use in targeting misfolded and/or aggregated SOD1 in bone and/or joints of a subject, preferably wherein the antibody is the AP-101 antibody or an miSOD1 binding fragment thereof, or wherein the anti-SOD1 antibody is the labeled antibody conjugate as described herein.

Particularly preferred, the anti-SOD1 antibody or an antigen binding fragment thereof for use in targeting is provided for targeting of misfolded and/or aggregated SOD1 in subjects affected by or having ALS. Furthermore, preferably the subject is a mammal, mostly preferred a human.

In addition, the present invention also related to an use of an labeled-antibody conjugate as defined herein for the manufacture of an imaging composition for use in the imaging methods as defined herein.

Furthermore, the present invention also provides a method for preparation of a solution comprising the ⁸⁹Zr-DFO-AP-101 antibody conjugate as described in detail in Example 9. The so prepared imaging solution will be used and tested in the clinical study as described in Example 8. Preferably, the method comprises the steps of:
(a) combining an ⁸⁹Zr-chloride solution with DFO-AP-101 to obtain a reaction solution. Preferably the ⁸⁹Zr-chloride solution is adjusted to a pH of 6.5-8.0. The adjustment is preferably performed by adding a sodium bicarbonate solution and HEPES buffer solution to obtain said intended pH of 6.5-8.0.
(b) warming the reaction solution to a temperature of about 37° C. for a period of about 45 minutes. Preferably, the warming of the solution takes place in a reactor, i.e., a controlled surrounding in which uniform temperature can be obtained and held for the required period.
(e) recovering ⁸⁹Zr-DFO-AP-101 as a recovery solution. Preferably the solution comprising ⁸⁹Zr-DFO-AP-101 is recovered into a container, e.g., a sealable vial.

The method preferably further comprises the following step (f) and/or step (g):
(f) adjustment of the ⁸⁹Zr-DFO-AP-101 recovery solution to a final volume by adding saline, preferably wherein the final volume is 10 mL. Preferably again 0.9% saline is used in this step;
(g) filtering of the ⁸⁹Zr-DFO-AP-101 solution.

Furthermore, the step of recovering of ⁸⁹Zr-DFO-AP-101 is preferably performed on a column comprising a gel filtration resin comprising the following steps:
(c) conditioning a gel-filtration resin in a column with 10 mL saline 0.9%, preferably at 1.5 mL/min; wherein preferably this step is repeated 3 times. Preferably a column filled with a gel-filtration resin is used in this step, wherein the resin is preferably Sephadex G-25. Mostly preferred a PD-10 column (GE Healthcare is used as the column in this step.
(d) transferring the reaction solution of (b) with 4 mL of saline to the conditioned column of (c)
(e) recovering ⁸⁹Zr-DFO-AP-101 from the reaction solution into a recovered solution from the column.

Accordingly, in a preferred embodiment, the above method is performed comprising the following steps:
(a) combining an adjusted to a pH of 6.5-8.0 ⁸⁹Zr-chloride solution with DFO-AP-101 to obtain a reaction solution;
(b) warming the reaction solution to a temperature of about 37° C. for a period of about 45 minutes;
(c) conditioning a gel-filtration resin in a column with 10 mL saline 0.9%, preferably at 1.5 mL/min; wherein preferably this step is repeated 3 times;
(d) transferring the reaction solution of (b) with 4 mL of saline to the conditioned column of (c); and
(e) recovering ⁸⁹Zr-DFO-AP-101 from the column as a recovery solution.

Preferably the method further comprises the following step (f) and/or (g)
(f) adjustment of the ⁸⁹Zr-DFO-AP-101 recovery solution to a final volume by adding saline, preferably wherein the final volume is 10 mL; and
(g) filtering of the ⁸⁹Zr-DFO-AP-101 solution.

Preferably the steps of are performed in the indicated order.

In a preferred embodiment, the method is further provided, wherein the ⁸⁹Zr-chloride solution of step (a) is provided by a method comprising the following steps:
(a1) providing a solution of ⁸⁹Zr-oxalate onto a quaternary methyl ammonium (QMA) anion exchange column;
(a2) adding a solution comprising HCl to the QMA anion exchange column; and
(a3) collecting the ⁸⁹Zr-chloride solution eluting from the column.

After performing the steps (a1) to (a3) the method for preparation of a solution comprising the ⁸⁹Zr-DFO-AP-101 antibody conjugate is continued at step (a) as defined above.

After the solution comprising the ⁸⁹Zr-DFO-AP-101 antibody conjugate has been obtained, the solution can be further processed to obtain a composition, which may be injected into the subject to perform the imaging methods as defined herein. Such a composition has then a shelf life of about 29 hours after the end of its synthesis. Therefore, preferably the method for preparation of a solution comprising the ⁸⁹Zr-DFO-AP-101 antibody conjugate is provided further comprising the step of:
(h) formulation of the filtered ⁸⁹Zr-DFO-AP-101 solution obtained from step (g) into a composition by addition of 0.9% saline and 10 mg of gentisic acid. Preferably the total volume of the formulated composition amounts to 10 mL.

Accordingly, in one preferred embodiment the method for preparation of a solution comprising the ⁸⁹Zr-DFO-AP-101 antibody conjugate is performed comprising the steps (a1)-(a3) and (a) to (h).

The obtained solution is preferably sterile and pyrogen fee and can be used as diagnostic agent for intravenous administration, preferably with 5 mg or less of ⁸⁹Zr-DFO-AP-101 per injection. The solution has a shelf-life of 29 hours after the end of synthesis (EOF). Molecular weight of ⁸⁹Zr-DFO-AP-101 is about 147,332-147,792 kDa. The ⁸⁹Zr isotope and its respective salts to be used in the method above can be prepared by a cyclotron via a ⁸⁹Y(p,n)⁸⁹Zr reaction as described in detail in Alnahwi et al. (2018), wherein the resulting ⁸⁹Zr-oxalate is transformed via the above method into the less toxic ⁸⁹Zr-chloride. As indicated above, of course also ⁸⁹Zr isotope, ⁸⁹Zr-oxalate and ⁸⁹Zr-chloride from other sources can be used in the method above to label DFO-AP-101 and generate the solution comprising ⁸⁹Zr-DFO-AP-101 for use in the intended clinical trial or generally in imaging methods. The particular steps at which the method above can be entered depending on the fact whether ⁸⁹Zr-oxalate or ⁸⁹Zr-chloride are used as starting material are indicated above, e.g., steps (a1) to (a3) may be skipped and the method started with step (a) if ⁸⁹Zr-chloride is already available for performing the method. No or few adverse effects are expected when the solution obtained by the method above or ⁸⁹Zr-DFO-AP-101 in general is used in tracer doses as required for imaging. This expectation bases on several factors. Animal experiments have shown that [⁸⁹Zr]Zr-DFO-AP-101 is stable in plasma and that it retained its affinity and specificity for misfolded and/or aggregated SOD1. It displayed a specific uptake into several animal regions of interest, including the spinal cord and vertebra of ALS model mice. The data obtained in the field with ⁸⁹Zr-DFO-antibodies in the past 10 years has shown that such antibodies are safe and well tolerated by patients, which lead to a great number of corresponding antibodies approved by the FDA (e.g., correspondingly labelled trastuzumab, bevacizumab, cetuximab and rituximab; see, e.g., Heskamp et al (2017), Stabin et al (1997), Laforest et al (2016), Dijkers et al (2010), Yoon et al (2020), Veldhuijzen van Zanten et al. (2018), Oosting et al. (2016), Even et al (2017), Makris et al (2015), (Jauw et al (2017) and Bruijnen et al (2016)). The clinical trials 1 and 2a described in Example 8 testing AP-101 did not show any antibody related adverse effects. Since the [⁸⁹Zr]Zr-DFO-AP-101 doses tested in the new clinical trial described in Example 8 are (with planned 1.5 mg dose per patient) up to 1666 times lower than the doses tested in said AP-101 trials (100 mg, 500 mg and 2500 mg doses) this also allows to expect that an equivalent safety profile will be observed. The DFO chelator used to conjugate the radioactive isotopes to the antibodies is known in the art, FDA approved and the data regarding its use in the field indicates that it is relatively safe and well-tolerated by patients as well. Accordingly, in sum it can be also expected that ⁸⁹Zr-DFO-AP-101 will also reveal itself as relatively safe and well tolerated by the patients.

### Terms

For the avoidance of any doubt, it is emphasized that the expressions “in some embodiments”, “in a certain embodiments,” “in certain instances,” “in some instances,” “in some aspects,” “in a further embodiment,” “in one embodiment,” “in a further aspect,” “in a first aspect,” “in a second aspect,” etc., and the like are used and meant such that any of the embodiments described therein are to be read with a mind to combine each of the features of those embodiments and that the disclosure has to be treated in the same way as if the combination of the features of those embodiments and aspects would be spelled out in one embodiment. The same is true for any combination of embodiments and features of the appended claims and illustrated in the Examples, which are also intended to be combined with features from corresponding embodiments disclosed in the description, wherein only for the sake of consistency and conciseness the embodiments are characterized by dependencies while in fact each embodiment and combination of features, which could be construed due to the (multiple) dependencies must be seen to be literally disclosed and not considered as a selection among different choices.

The terms “radionuclide”, “radioactive nuclide”, “radioisotope” or “radioactive isotope” are used interchangeably herein for a nuclide that has excess nuclear energy, making it unstable and wherein the excess energy is emitted, e.g., as gamma rays and/or as particles. Preferably the emission takes place at least in part as particles such as beta particles (also called beta ray or beta radiation), mostly preferred as positrons.

The terms “fragment,” “variant,” “derivative” and “analog” when referring to antibodies or antibody polypeptides of the present invention include any polypeptides which retain at least some of the antigen-binding properties of the corresponding native binding molecule, antibody, or polypeptide. As indicated above, the variable region allows the antibody to selectively recognize and specifically bind epitopes on antigens. That is, the V_{L} domain and V_{H} domain, or subset of the complementarity determining regions (CDRs), of an antibody combine to form the variable region that defines a three-dimensional antigen-binding site. This quaternary antibody structure forms the antigen-binding site present at the end of each arm of the Y. More specifically, the antigen-binding site is defined by three CDRs on each of the V_{H} and V_{L} chains. Any antibody fragment which contains sufficient structure to specifically bind to misfolded SOD1 is denoted herein interchangeably as a “binding fragment” or an “immunospecific fragment.” Antibodies or antigen-binding fragments, variants, or derivatives thereof and equivalent binding molecules of the invention include, but are not limited to, polyclonal, monoclonal, multispecific, human, humanized, primatized, murinized or chimeric antibodies, single chain antibodies, epitope-binding fragments, e.g., Fab, Fab′ and F(ab′)₂, Fd, Fvs, single-chain Fvs (scFv), single-chain antibodies, single domain antibodies, disulfide-linked Fvs (sdFv), fragments comprising either a V_{L} or V_{H} domain and fragments produced by a Fab expression library. ScFv molecules are known in the art and are described, e.g., in U.S. Pat. No. 5,892,019.

By “specifically binding”, or “specifically recognizing”, used interchangeably herein, it is generally meant that a binding molecule, e.g., an antibody or fragment thereof binds to an epitope via its antigen-binding domain, and that the binding entails some complementarity between the antigen-binding domain and the epitope. According to this definition, an antibody is said to “specifically bind” to an epitope when it binds to that epitope, via its antigen-binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D”. The reference that an antibody or fragment thereof binds to misfolded SOD1 as used in the present application is used in the meaning used for the term “specifically binding” or “specifically recognizing” as explained above. The term “binding” when referring to an antibody or fragment thereof as defined herein and its capability to bind misfolded SOD1 is used interchangeably and including the meaning of the terms “specifically binding” or “specifically recognizing” as defined above.

The term “detection label” refers to a chemical moiety that can be covalently attached to an antibody and that functions to provide a detectable signal. Examples of such labels include fluorescent moieties, chemiluminescent moieties, bioluminescent moieties, nanoparticles, magnetic particles, metal-containing particles, and radiolabels, such as radionuclides.

The term “bifunctional chelator” refers to a chemical moiety that attaches an antibody or antibody fragment to a label, e.g., a radionuclide. Bifunctional chelators include 1) a chelator moiety functional to bind a (radionuclide) label and 2) a reactive functional group. Bifunctional chelators function by complexing a label (e.g., radionuclide) with the chelator moiety and by covalently attaching the chelator moiety (and complexed radionuclide) to an antibody or antibody fragment via reaction of the reactive functional group with a corresponding reactive functional group of the antibody (or its fragment). Examples of suitable reactive functional groups capable of attaching a chelator moiety an antibody or fragment thereof, e.g., to a primary amine group, a hydroxyl group, and/or a cysteine amino acid are known to those of skill in the art (see e.g., Denardo et al., 1998, Clin Cancer Res. 4(10):2483-90; Peterson et al., 1999, Bioconjug. Chem. 10(4):553-7; and Zimmerman et al., 1999, Nucl. Med. Biol. 26(8):943-50).

The transgenic mice expressing mSOD1 are referred to interchangeably herein and in the Figures as Tg or ALS, wild type mice are referred to interchangeably herein as Wt or CTL.

By “subject”, “individual” or “patient” is meant any subject, in particular a mammalian subject, e.g., a human patient (e.g., a patient having ALS) for whom diagnosis, prognosis, prevention or monitoring of the therapy is desired. The terms “subject”, “individual” and “patient” are used interchangeable herein.

As used herein, the term “about” used in the context of quantitative measurements means the indicated amount±10%. For example, with a ±10% range, “about 5 mg” can mean 4.5 to 5.5.

As used herein, the terms “treat” or “treatment” refer to both therapeutic treatment and prophylactic or preventative measures, in which the object is to prevent or slow down (lessen) an undesired physiological change or disorder, such as the development of cardiac deficiency. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. Those in need of treatment include those already with the condition or disorder as well as those prone to have the condition or disorder or those in which the manifestation of the condition or disorder is to be prevented.

Pharmaceutically acceptable carriers and administration routes can be taken from corresponding literature known to the person skilled in the art. The pharmaceutical compositions of the present invention can be formulated according to methods well known in the art; see for example Remington: The Science and Practice of Pharmacy (2000) by the University of Sciences in Philadelphia, ISBN 0-683-306472, Vaccine Protocols. 2nd Edition by Robinson et al., Humana Press, Totowa, New Jersey, USA, 2003; Banga, Therapeutic Peptides and Proteins: Formulation, Processing, and Delivery Systems. 2nd Edition by Taylor and Francis. (2006), ISBN: 0-8493-1630-8. Examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by well-known conventional methods. These pharmaceutical compositions can be administered to the subject at a suitable dose. Administration of the suitable compositions may be affected in different ways. Examples include administering a composition containing a pharmaceutically acceptable carrier via oral, intranasal, rectal, topical, intraperitoneal, intravenous, intramuscular, subcutaneous, subdermal, transdermal, intrathecal, and intracranial methods. Aerosol formulations such as nasal spray formulations include purified aqueous or other solutions of the active agent with preservative agents and isotonic agents. Such formulations are preferably adjusted to a pH and isotonic state compatible with the nasal mucous membranes. Pharmaceutical compositions for oral administration, such as single domain antibody molecules (e.g., “Nanobodies™”) etc. are also envisaged in the present invention. Such oral formulations may be in tablet, capsule, powder, liquid or semi-solid form. A tablet may comprise a solid carrier, such as gelatin or an adjuvant. Formulations for rectal or vaginal administration may be presented as a suppository with a suitable carrier; see also O'Hagan et al., Nature Reviews, Drug Discovery 2(9) (2003), 727-735. Further guidance regarding formulations that are suitable for various types of administration can be found in Remington's Pharmaceutical Sciences, Mace Publishing Company, Philadelphia, PA, 17th ed. (1985) and corresponding updates. For a brief review of methods for drug delivery see Langer, Science 249 (1990), 1527-1533.

Furthermore, unless stated otherwise, terms and expressions used herein in order to characterize the present invention are given in the definitions as provided in WO 2012/080518 A1, in particular in subsection “I. Definitions” at pages 10 to 30, the disclosure content of which is explicitly incorporated herein by reference. The same applies to the general embodiments disclosed in WO 2012/080518 A1 for antibodies, etc.

Several documents are cited throughout the text of this specification. The contents of all cited references (including literature references, issued patents, published patent applications as cited throughout this application including the background section and manufacturer's specifications, instructions, etc.) are hereby expressly incorporated by reference; however, there is no admission that any document cited is indeed prior art as to the present invention.

A more complete understanding can be obtained by reference to the following specific examples which are provided herein for purposes of illustration only and are not intended to limit the scope of the invention.

### EXAMPLES

The overall objective of the experiments described hereinbelow was to develop and validate a radiotracer derivatized from AP-101 and labeled with ⁸⁹Zr for detection of misfolded SOD-1 deposits via PET imaging. Such a radiotracer would find application in the early diagnosis of ALS, in the follow-up of the therapeutic response and/or in monitoring the disease, and as such be the first diagnostic tool available for ALS.

The specific aims of the experiments described herein below were:

Aim-1 To prepare the ⁸⁹Zr-based PET tracers derivatized from AP-101 and to preferably to develop an automated process for the production of the selected ⁸⁹Zr-DFO-AP-101 PET tracer: Automation of ⁸⁹Zr-DFO-AP-101 using a cassette-based module will reduce dosimetry to the operator, allowing for reproducibility and enabling the GMP-compliant quality standards. Two ⁸⁹Zr-AP-101 derivatized PET tracers were developed; ⁸⁹Zr-DFO-AP-101 and the deglycosylated ⁸⁹Zr-DFO-AP-101 (Degly-⁸⁹Zr-DFO-AP-101) to reduce the uptake of the radioimmunoconjugate in nontarget tissues such as the liver and spleen by immune cells expressing Fc-7-receptors (FcγR).

Aim-2 To assess the efficacy of ⁸⁹Zr-DFO-AP-101 for early detection of aggregated SOD1 by PET imaging in an appropriate animal model. A longitudinal imaging study was performed to identify the optimal mice age and time post administration of ⁸⁹Zr-DFO-AP-101 for the detection misfolded SOD1 aggregation in symptomatic and pre-symptomatic transgenic (Tg, [B6.Cg-Tg(SOD1*G93A)1Gur/J)]) mice expressing the formation of m-SOD1 aggregate. Control imaging experiments were done with ⁸⁹Zr-DFO-AP-101 and Degly-⁸⁹Zr-DFO-AP-101 in wild type (Wt) mice.

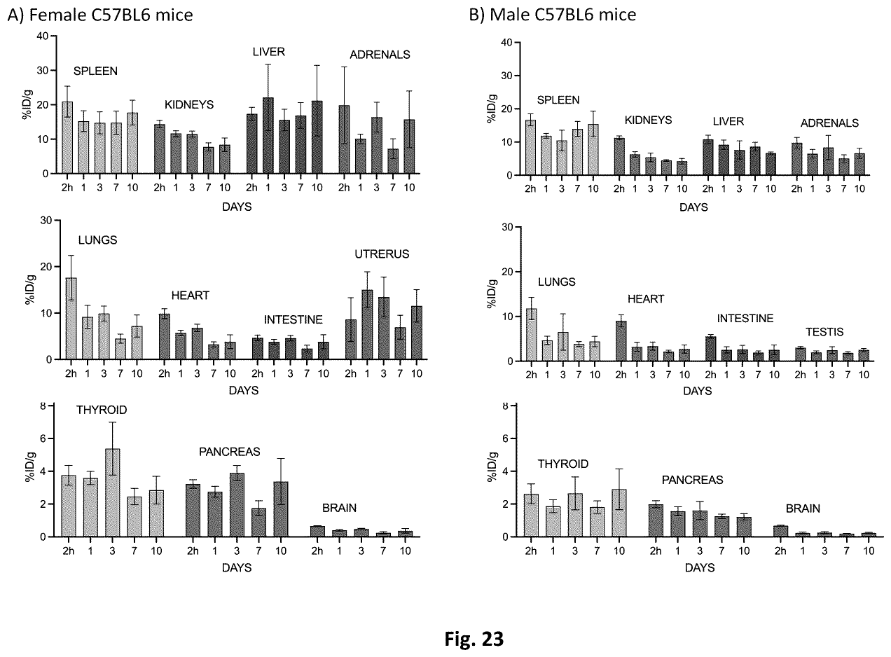

Aim-3 To determine if ⁸⁹Zr-DFO-AP-101 and Degly-⁸⁹Zr-DFO-AP-101 can be used for early diagnosis of ALS and in the follow-up of the therapeutic response; B) to estimate the anticipated human dose (OLINDA) of the selected ⁸⁹Zr-DFO-AP-101 PET tracer. A blocking study using unlabeled AP-101 (100 mg/Kg) was performed to evaluate the impact of unlabeled AP-101 on the bioavailability and the capacity of ⁸⁹Zr-DFO-AP-101 to bind to mSOD1 aggregates. A dosimetry study in Wt mice was carried with Degly-⁸⁹Zr-DFO-AP-101 to estimate the expected injected dose in humans for diagnostic use with PET. Mice of the C57BL6 strain were used as model mouse. Measured effective doses in human estimated for ⁸⁹Zr-DFO-AP-101 are 0.171 mSv/MBq and 0.237 mSv/MBq in adult male and female, respectively (see FIG. 23 and description of the dosimetry experiments in mice in Example 8). Based on the preliminary results from the phase I clinical trial described in Example 10, the effective doses in humans for ⁸⁹Zr-DFO-AP-101 are estimated depending on the gender as within the range from about 0.4 mSv/MBq to about 0.65 mSv/MBq, in particular an effective dose of 0.48 mSv/MBq in human male model and of 0.54 mSv/MBq in female human model.

Hereinbelow, the preparation and the characterization (stability, shelf live) of ⁸⁹Zr-DFO-AP-101 and its analog Degly-⁸⁹Zr-DFO-AP-101 and the assessment of their potential for the detection of mSOD1 aggregates by PET imaging is described.

### Materials and Methods

### Preparation of DFO-AP-101

Sample preparation. AP-101 (20 mg/mL, 1 mL) was centrifuged to remove all the additives using four 50 kDa molecular weight cut off centrifugal filter units (Amicon® Ultra-05) and washed with PBS (trace metals basis, 300 μL each) pH=7.2 at 5000 rpm for 10 min four times. The final wash was done with NaHCO₃ 0.1 M, pH=8-9. The samples (~100-150 μL) were then combined in another 50 kDa molecular weight cut off centrifugal filter unit (Amicon® Ultra-05) and centrifuged to reach a final volume of ~150-200 μL. The mAb AP-101 was diluted with a NaHCO₃ 0.1 M solution to reach a volume of ~500-600 μL and transferred into a 5 mL screw thread vial (Fisherbrand) using a 200 μL micropipette. Concentrations lower than 2 mg/mL decreased the efficiency and the reproducibility of the conjugation reaction, accordingly, concentrations of AP-101 at 2 mg/mL and higher were used.

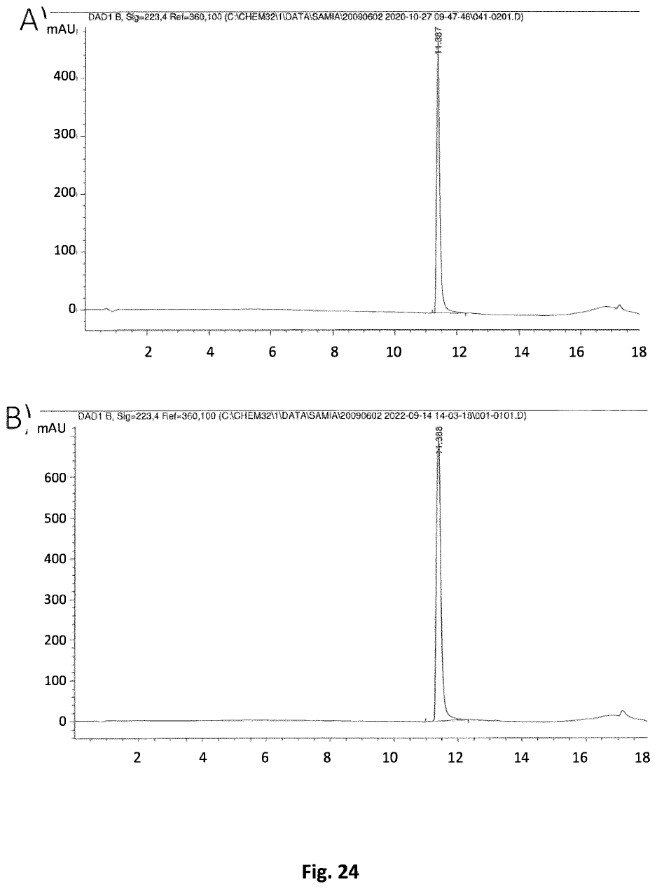

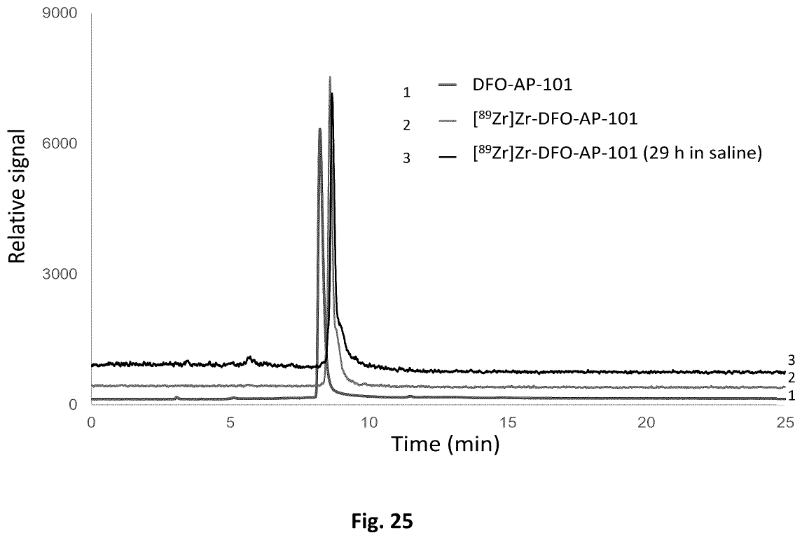

DFO Conjugation. The AP-101 solution (500-600 μL in NaHCO₃ 0.1 M) was incubated with three equivalents of SCN-Bz-DFO (15 μL of a solution of 1.1 mg of SCN-Bz-DFO dissolved in 100 μL of DMSO). The resulting mixture was kept for 1 hour at 37° C. The DMSO concentration was kept below 2% to avoid change in the properties of AP-101. The conjugate was then purified ten times using the centrifugal filter technique described above using 2 centricons and PBS (trace metals basis, 200 μL each) pH=7.2. The samples (~100-150 μL) were then combined into a 5 mL screw thread vial (Fisherbrand) and stored at 4° C. HPLC on Size Exclusion Chromatography (SEC) was performed on a BioSep-SEC S3000 column using standard mobile phase (0.1 M PBS+0.025% NaN3) with a flow rate of 1 mL/minute at 280 nm. The determination of the average number of DFO per molecule of AP-101 was done using a modified isotopic dilution assay as described by Holland et al. J Nucl Med. (2010); 51(8):1293-1300 in particular in Supplemental Material at pages 33/46, which document (including its Supplemental Material) is herewith incorporated herein in its entirety. Concentration of the conjugate was measured spectrophotometrically using the Bicinchoninic Acid (BCA) protein assay (He. F. (2011) Bio-101: e44. DOI: 10.21769/BioProtoc.44), which document is herewith incorporated herein in its entirety. The half-life of AP-101 in serum is about 9.5 days, the shelf-life of DFO-AP-101 is almost 2 years at 2-8° C., as seen in the HPLC profile when performed direct after purification and 2 years later (see FIG. 24).

### Preparation for the Clinical Study as Described in Example 8

Antibodies or antibody-conjugate labeled with ⁸⁹Zr can be obtained, e.g., by conjugating ⁸⁹Zr obtained initially as ⁸⁹Zr-oxalate as described in Alnahwi et al. (2018), in particular by the methods described in the 2. Materials and Methods section at pages 2-6 therein, which disclosure is herewith incorporated herein, to the antibody or antibody conjugate to be labelled, wherein preferably the antibody conjugate is DFO-AP-101. The ⁸⁹Zr-oxalate, e.g., obtained by said method, is preferably first trapped on a quaternary methyl ammonium (QMA) anion exchange column. To automate the process, the subsequent steps can be performed, e.g., on a miniAllinOne (MiniAIO) cassette-based module (Ans, Belgium), also described in Alnahwi et al. (2018), specifically in section 2.4. Automated Cassette-Based Module and Separation Chemistry for ⁸⁹Zr-oxalate, disclosure of which section is herewith incorporated herein.

Such an automated production method preferably comprises the following steps. The ⁸⁹Zr-oxalate is transferred (trapped) onto an anion exchange column, e.g., a quaternary methyl ammonium (QMA) column. A solution comprising HCl is then added to the column, to transform ⁸⁹Zr-oxalate to less toxic ⁸⁹Zr-chloride and to elute it from the column. Preferably so called HCl metal trace is used for this aim, i.e., a 99.999% pure HCl solution based on trace metal analysis. Preferably a NaCl—HCl solution is used for this aim, wherein mostly preferred the solution is a 500 mM NaCl-137 mM HCl solution. The volume of said NaCl—HCl solution is preferably about 0.25 mL to about 2.5 mL, more preferred about 0.5 mL to about 1.5 mL or 1 mL, mostly preferred about 0.5 mL. The ⁸⁹Zr-chloride comprising eluate is received from the column in a container, e.g., a vial. If ⁸⁹Zr-chloride or another ⁸⁹Zr-salt which is less toxic than ⁸⁹Zr-oxalate is available from other sources, the herein described method can be also performed starting with the following step. The vial/container is then transferred to a reactor, i.e., a place in which in particular the temperature of the subsequent reaction can be controlled. The pH of the eluate is adjusted to obtain a pH in the range of about 6.5-8.0. For adjusting the pH preferably a sodium bicarbonate solution and HEPES buffer solution are used. The preferred antibody or antibody conjugate, preferably DFO-AP-101 is also added to the pH adjusted eluate. The amount of the antibody or antibody conjugate can be chosen based on the amount of ⁸⁹Zr-oxalate used in the reaction. For example, about 1 mg to about 6 mg, about 2 mg to about 5 mg, about 2.5 mg to about 4 mg, about 3 mg to about 3.5 mg of the antibody or antibody conjugate, preferably of DFO-AP-101 are added. The components in the resulting solution are then warmed in the reactor for a period of time. The resulting solution is warmed in the reactor from about 34° C. to about 42° C., preferably from about 35° C. to about 40° C., more preferred from about 36° C. to about 38° C. for a period of about 15 minutes to about 90 minutes, preferably from about 20 minutes to about 60 minutes, more preferred from about 30 minutes to about 50 minutes, even more preferred from about 40 minutes to about 50 minutes. Mostly preferred the resulting solution is warmed to about 37° C. for about 45 minutes. During the solution is warmed in the reactor another desalting size-exclusion column is conditioned. Preferably a column filled with a gel-filtration resin is used in this step, wherein the resin is preferably Sephadex G-25. Mostly preferred a PD-10 column (GE Healthcare) is used in this step. Conditioning comprises rinsing of the column with saline (mixture of NaCl with water), wherein the rinsing is preferably performed repeatedly, mostly preferred 3 times. Preferably 0.9% saline is used, wherein the rinsing occurs preferably at 1.5 mL/min. The amount of the saline is preferably about 10 mL per rinse. After the radiolabeling reaction in the reactor, the solution with the so generated ⁸⁹Zr-DFO-AP-101 is transferred to the conditioned column and eluted therefrom again with saline. The amount of the saline used for elution should be below 10 mL, preferably 4 mL of saline (0.9%) are used. The eluate is collected in a new vial and the obtained eluate volume can be adjusted to an intended end volume. Preferably the collected ⁸⁹Zr-DFO-AP-101 solution is adjusted by adding saline to a final volume of about 10 mL. Thereafter the ⁸⁹Zr-DFO-AP-101 solution is filtered and may be used for the intended administration to patients. For this aim, the ⁸⁹Zr-DFO-AP-101 solution is preferably formulated in a vehicle containing 0.9% saline and 10 mg gentisic acid to prevent radiolysis. In experiments performed in accordance with the present invention, the so obtained ⁸⁹Zr-DFO-AP-101 has shown a high radiochemical purity of 90% and above with radiochemical yields greater than about 60%. Radiochemical impurities as ⁸⁸Zr and ⁸⁸Y were at very low level and below 25 within the 29 hours of end-of-synthesis (EOS) shelf-life.

### Preparation of Degly-DFO-AP-101

To generate deglycosylated DFO-AP-101, the immobilized GlycINATOR spin columns containing the GlycINATOR enzyme covalently coupled to agarose beads GlycINATOR® (EndoS2) was used for the removal of N-glycans. After equilibration of each column with 3×300 μL of 1M PBS (trace metal) and centrifugation at 200 g for 1 min, DFO-AP-101 (3 mg, 300 μL) was added to the column (0.5 mg per column). The volume of each column was adjusted to 300 μL of PBS and the solution is incubated at room temperature for 2 h by end-over-end mechanical mixing. After centrifugation at 1000 g for 1 min, Degly-DFO-AP-101 was collected in different reaction vials/tubes. For maximum yield, 300 μL of 1 M PBS was added to GlycINATOR spin columns followed by centrifugation for recovering residual Degly-DFO-AP-101, this step was repeated 3 times. After GlycINATOR digestion, HPLC on SEC was performed as described above to characterize the crude deglycosylated antibody. The conjugate was then purified three times using the centrifugal filter technique described above using 2 centricons and PBS (trace metals basis, 300 μL each) pH=7.2. The samples (~100-150 μL) were then combined in another 30 kDa molecular weight cut off centrifugal filter unit (Amicon® Ultra-05) and centrifuged to reach a final volume of ~150-200 μL. The samples (~100-150 μL) were then combined into a 5 mL screw thread vial (Fisherbrand) and stored at 4° C. Concentration of the conjugate was measured spectrophotometrically using the BCA protein assay (He. F. 2011). The determination of the average number of DFO per molecule of AP-101 was done using a modified isotopic dilution assay as described by Holland (Holland et al. 2010), which document is herewith incorporated herein in its entirety. The specific activity was determined following our previous reported procedure (Alnahwi et al. 2018), which document is herewith incorporated herein in its entirety.

### Radiolabeling and Determination of the Average Number of Chelators Per Antibody

Typically, 1-2 GBq of ⁸⁹Zr(oxalate)2 were produced in a cyclotron facility following the procedure described at pages 2-9 of the article by Alnahwi et al., Appl. Sci. 8 (2018):1579 (Alnahwi et al., 2018—included herein in its entirety) and using ⁸⁹Y-pressed targets. ⁸⁹ZrCl₄ was loaded on the ion exchange cartridge and eluted as ⁸⁹ZrCl₄ in 0.5-1 mL of 1 M HCl with a 95% recovery yield. 400 μL of ⁸⁹ZrCl₄ (400-500 MBq) was neutralized with 110-120 μL of 2 M Na₂CO₃ to adjust the pH of ⁸⁹ZrCl₄ solution at 7-7.5. To the neutralized ⁸⁹ZrCl₄ solution, DFO-AP-101 (0.5-1 mg) in 0.5 M NaOAc pH=6.5 was added and the volume adjusted to obtain at least a concentration of 0.2 μg/μL of DFO AP-101 with a final pH of 7-7.2. The radiolabeling was allowed to progress for 45 min at 37° C. The radiolabeling yield of the reaction was assessed by radio-TLC using 50 mM DTPA (pH 5-6) as mobile phase and counted on a TLC plate reader. Final radiochemical purity was determined using radio-TLC and SDS-PAGE.

### Stability Studies

Shelf life of the PET tracers. Stability studies were performed on formulated ⁸⁹Zr-DFO-AP-101 and Degly-⁸⁹Zr-DFO-AP-101 (in 1M PBS, pH 7.2 alone or in the presence of gentisic acid (GA, 2.5 mg/mL) kept at 4° C. over 9-10 days. The presence of free ⁸⁹Zr was monitored by radio-TLC. The solution was analyzed by SDS-PAGE and exposed to autoradiography film to detect ⁸⁹Zr metabolites and/or aggregates.

In vivo stability studies. ⁸⁹Zr-DFO-AP-101 (175-200 MBq) in PBS were also incubated at 37° C. in 500 μL of fresh mouse plasma and PBS (1/1) for different periods of time. The stability was directly monitored by radio-ITLC using 50 mM DTPA as eluant to follow the presence of free ⁸⁹Zr.

### SDS-PAGE Analysis

DFO-AP-101 and Degly-DFO-AP-101 were characterized via sodium dodecyl sulfate-polyacrylamide gel electrophoresis (SDS-PAGE). Briefly, 4-20 ng antibody (6-30 μL of a 0.6 mg/mL stock) were combined with 0-16 μL of Tris/glycine/SDS buffer and 4 μL 4× Laemmli protein sample buffer. For the denaturing conditions, 1 μL of 1,4-dithiothreitol (DTT) was added and this mixture was then denatured by heating in boiling water for 5 min using a heat block. Subsequently, each sample was then loaded alongside an appropriate molecular weight marker (PageRuler™ Prestained Protein Ladder, ThermoFisher Scientific) onto a 4-15% precast polyacrylamide gel (Bio-Rad) and run for 1 h at 120 volts in Tris/Glycine/SDS buffer. The completed gel was stained using Coomassie Blue solution for 1 h, and destained overnight in destaining solution (10% acetic acid, 10% methanol). For ⁸⁹Zr-DFO-AP-101 and Degly-⁸⁹Zr-DFO-AP-101, the radioactive gel, was placed without coloration on a sheet of Saran wrap and exposed to a phosphor screen (Molecular Dynamics) for 30-60 min. The screen was then read by a Storm phosphor Imager (Amersham Biosciences). The image was analyzed with Fiji software and the protein ladder picture from the gel was overlaid on the image for analysis.

### Animal Care and Experimental Conditions

The animal study was approved by the University of Sherbrooke IACUC and was conform to the Canadian Council on Animal Care (CCAC) guidelines. Female Tg [B6.Cg-Tg (SOD1*G93A) 1Gur/J)] mice expressing the ALS genotype and their controls Wt [C56BL/6J] were purchased from Jackson (610 Main Street Bar Harbor, ME 04609). The mice were between 51 and 58 days old at arrival and were hosted at CIMS animal care facility until they were 126 days old. Upon arrival. The mice were examined to ensure that they were healthy and that each group was uniform in appearance and size. Throughout the study, the mice were housed by groups of maximum 4 mice per cage on a ventilated rack (Techniplast SealSafe Plus. Techniplast Ventilation Unit) in a controlled environment room. Cages were lined with a corn cob litter and included enrichment (Plastic dome. EnviroDri® nesting material and wood block). Cages were changed once per week. Throughout the study the mice had free access to (standard tab) water and food (LabDiet 5008). Throughout the study the mice were observed daily to detect the expression of ALS symptoms (weight loss, suspension, and walking tests as described in Hatzipetros et al., J Vis Exp. 104 (2015):53257.

### Radiotracer Administration

Under isoflurane anesthesia. (Induction 2-3% isoflurane, maintenance 1-2.5%, 1-1.5 L of oxygen/min) a catheter (30G needle/PE10 tube) was installed within the tail vein of the subject mouse for the administration of the radiotracer (20-30 MBq/0.2 mL saline 0.9%), either ⁸⁹Zr-DFO-AP-101, ⁸⁹Zr-DFO-AP-101 co-injected with 100 mg/kg of AP-101 or Degly-⁸⁹Zr-DFO-AP-101. Imaging days post administration of the radiotracer were optimized as the first results were collected, the last groups being imaged only at days 7 and 10. Once the radiotracer administrated, the mouse was placed in its cage to recuperate from anesthesia and returned to the housing facility.

### PET Imaging and Image Analysis

PET imaging was performed on days 7 and 10 following the administration of the radiotracer. Imaging was done on a LabPET-2 PET scanner (https://imagingrt.com/. Bore size: 72 mm. Axial FOV: 50 mm. Transaxial FOV: 60 mm. Spatial resolution: 0.74 mm (Isotropic at 5 mm from center). Detection efficiency (250-650 keV): 3.3% dedicated to small animal imaging. Preparation and PET imaging was done under isoflurane anesthesia delivered through a nose cone (Induction 2-3% isoflurane then 1-2.5%. 1-1.5 L of oxygen/min) the level of anesthesia being modulated if required during PET imaging. An ophthalmic gel was applied to the eyes to prevent eye dryness. The mouse was positioned within the field of view of the PET imager and a 30 min static acquisition was performed then the bed of the imager was moved within the CT module of the LabPET8 scanner, and a 2 min CT acquisition was performed. During PET imaging, the mouse was kept warm using pulsed hot air maintained at 32° C., the environment temperature and respiration rate being monitored throughout the imaging procedure (PCSAM. Model 1025T Monitoring & Gating System, SA Instruments Inc.). Once the imaging procedure was completed, the mouse was placed in its cage to recuperate from anesthesia and returned to the housing facility.

### Aggregate Identification and Data Processing

PET data were acquired in list mode and reconstructed on a 200×200×168 grid with 0.3×0.3×0.3 mm³ voxel size using 16 iterations and 8 subset of the 3D Ordered Subset Expectation Maximization (OSEM) algorithm. All PET images were corrected for the physical radionuclide decay of ⁸⁹Zr and random coincidences. Using PMOD software (version 3.8, PMOD Technologies Ltd., Zurich, Switzerland), the PET image of each animal was co-registered with its corresponding CT image by a rigid matching transformation.

The steps used to identify mSOD1 aggregates and quantify their radiotracer content are presented in FIG. 1. Once reconstructed the PET images were visually inspected to identify potential mSOD1 aggregates, which appear as discrete radiotracer spots within the spinal cord. If the spots were considered valid, a 3D region of interest (ROI) was drawn to include the whole mSOD1 spots. The criteria for validity were that the spot was well defined within the spinal cord and not contaminated by radioactivity spilled in form the presence of the radiotracer in the anterior portion of the vertebra. Once validated the radiotracer content of the mSOD1 spot was expressed in terms of % ID/g. The weight in gram of mSOD1 aggregates was estimated from the ROI volume assuming a density of 1 g/mL. Next the spot radiotracer content was compared to that found in a similar ROI drawn within the same mouse (internal reference) and to an ROI traced in the same location in the control mice from the same group (external reference). From each experimental group (⁸⁹Zr-DFO-AP-101, ⁸⁹Zr-DFO-AP-101 blocked with 100 mg/kg AP-101 and deglycosylated ⁸⁹Zr-DFO-AP-101), the following data were reported: Number of mice in the group, total number of spots identified, number of mice with spots, percent of mice with spots, number of spots per mouse, spots intensity (% ID/g), internal reference intensity (% ID/g), external reference intensity (% ID/g), spot to internal and external reference ratio. The mSOD1 spot to internal ratio is considered the most pertinent value as it represents the capacity to discriminate mSOD1 spots from their true background.

The average radiotracer content in the spinal cord and the anterior vertebra bone was measured by tracing thin ROI going through the length of these tissues (FIG. 2). From these profile ROI, the average % ID/g values measured within the spinal cord and the ventral vertebra bone were compared along with that of the “Spinal cord to Vertebra ratio”. Other ROIs were drawn in the head of the femur and spleen to evaluate the average % ID/g values in these tissues following injection with ⁸⁹Zr-DFO-AP-101 and Degly-⁸⁹Zr-DFO-AP-101 (FIG. 3).

### Correction for Spill in Effect

Once located region of interest (ROI) was then traced around these spots and the measure radiotracer content corrected for the partial volume effect (PVE) using the recovery coefficient and for spill-in from the presence of free ⁸⁹Zr in the anterior part of the vertebra (FIG. 4). The recovery coefficient was obtained using the Ultra-Micro hot spot phantom (FIG. 5). The radioactivity content of m-SOD1 aggregated was expressed in terms of % ID/g.

### Biodistribution

After the last PET acquisition (Day 10), the mice were perfused to remove blood and the spinal cord and tissues of interest were removed to measure their radioactivity content. Under isoflurane anesthesia, the mouse was placed at a 45° angle. head up at the perfusion board set within blood collection dish. The thorax was then opened to get access to the heart, a catheter (25 G) was installed within the left ventricle to deliver the perfusion solution (PBS with heparin 10 U/mL) by means of a perfusion pump (20 mL @5 mL/min) and the right auricle was lacerated using a small scissor. The sampled tissues were rinsed with physiological saline, blotted dry and weighted. Their radioactivity content was then measured using a Hidex (HIDEX AMG, Gamble Technologies Limited, Mississauga, Canada) gamma counter. The measures were then corrected for radioactive decay. The results were expressed in units of percentage of injected dose per gram of tissue (% ID/g). The spinal cords and brains were preserved in (PFA 4% for 24 h and PBS for storage) and used for histopathological analysis.

### Example 1:—Synthesis and Characterization of ⁸⁹Zr-DFO-AP-101 and Degly ⁸⁹Zr-DFO-AP-101

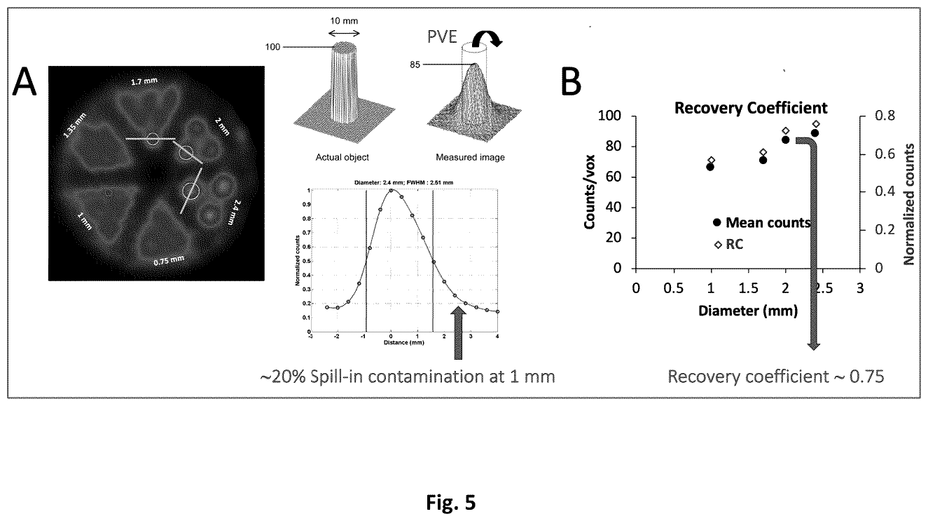

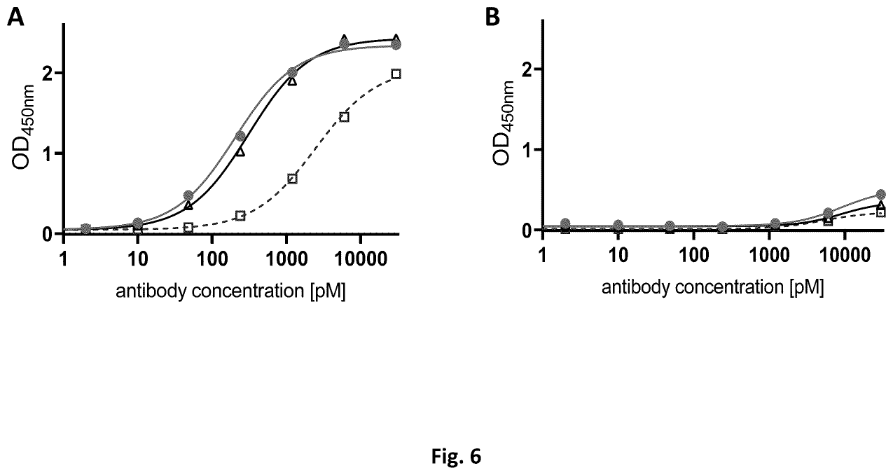

A large batch of DFO-AP-101 was prepared by optimizing the ⁸⁹Zr-chelator (DFO-SCN) conjugation on AP-101 lysine residues to obtain a DFO:AP-101 ratio of 1.1:1.0 (Table 1, n=7). The ^{nat}Zr-DFO-AP-101 conjugate maintained a high selectivity and affinity (EC₅₀=0.5 nM) for misfolded SOD1 and similar to non-modified AP-101 (FIG. 6). The purity of DFO-AP-101 was confirmed by HPLC analysis where a narrow peak was observed for the conjugate (FIG. 7 panel A). Absence of aggregates was confirmed by SDS-PAGE analysis (FIG. 7 panel B). The radiolabeling yield was greater than 97% with an apparent specific activity of 0.66 GBq/mg.

TABLE 1
Characterization of DFO-AP-101 and Degly-DFO-AP-
101 (see also FIG. 6 in regard of EC₅₀ values)

| | Ratio | Specific activity | EC₅₀ |
|---|---|---|---|
| Compound | DFO:AP-101 | (GBq/mg) | (nM) (mSOD1) |
| DFO-AP-101 | 1.1:1.0 | 0.66 ± 0.11 (n = 7) | 0.3 |
| Degly-DFO-AP-101 | 0.6:1.0 | 0.32 ± 0.05 (n = 2) | 2.4 |

To reduce the uptake of the radioimmunoconjugate in nontarget tissues such as the liver and spleen by immune cells expressing FcγR, the preparation of the deglycosylated analog of DFO-AP-101 was considered. The deglycosylation was performed on the DFO-AP-101 using EndoS2 kit (deGlycIT spin columns of the company Genovis AB, Sweden comprising an IgG-specific endoglycosidase acting on complex type N-glycans at the Fc-glycosylation site of IgG, wherein the endoglycosidase is covalently coupled to agarose beads). The loss of DFO chelator was observed during the deglycosylation: the resulting Degly-DFO-AP-101 has an average of 0.6 DFO per antibody as estimated by isotopic dilution techniques (Table 1). No trace of aggregate was observed on the purified Degly-DFO-AP-101 (FIG. 7 panel B). The removal of glycans was monitored using gel electrophoresis under reducing conditions and confirmed by a downward shift in the mobility of the immunoglobulin heavy chains at ~55 kD (FIG. 7 panel B). High radiolabeling yield and apparent specific activity were achieved. Specific activity of ⁸⁹Zr Degly-DFO-AP-101 (0.32±0.11 GBq/mg) was slightly lower than that of ⁸⁹Zr-DFO-AP-101. This can be explained by the lower DFO:AP-101 ratio. Degly-^{nat}Zr-DFO-AP-101 has a lower affinity (EC₅₀=2.4 nM) for misfolded SOD1 compared to non-modified AP-101, but its EC50 value is similar to that of ^{nat}Zr-DFO-AP-101

### Example 2: Stability of Degly ⁸⁹Zr-DFO-AP-101

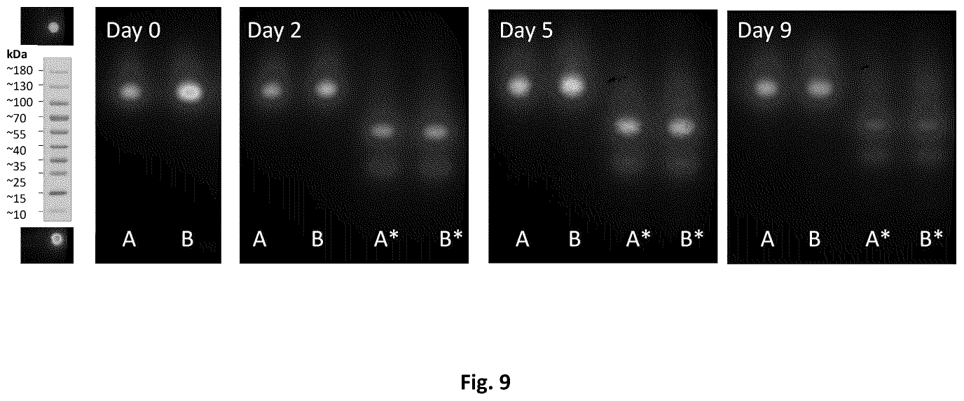

⁸⁹Zr-DFO-AP-101 is stable at 4° C. for over 10 days when formulated in PBS and for 7 days in mouse plasma. Only trace of free ⁸⁹Zr (<5%) was detected after 10 days incubation in plasma. Degly-⁸⁹Zr-DFO-AP-101 was also stable at 4° C. in PBS with and without the presence of GA, which was used for its antioxidant and radioprotective properties (Joshi et al. 2012) (FIG. 9). One should note that the presence of ⁸⁹Zr-DFO chelator was mainly found on the immunoglobulin heavy chains at ~55 kD of the Degly-⁸⁹Zr-DFO-AP-101.

### Example 3: Assessing the Feasibility of Using ⁸⁹Zr-DFO-AP-101 and Degly-⁸⁹Zr-DFO-AP-101 for PET Imaging of Spinal Cord mSOD1 Aggregates

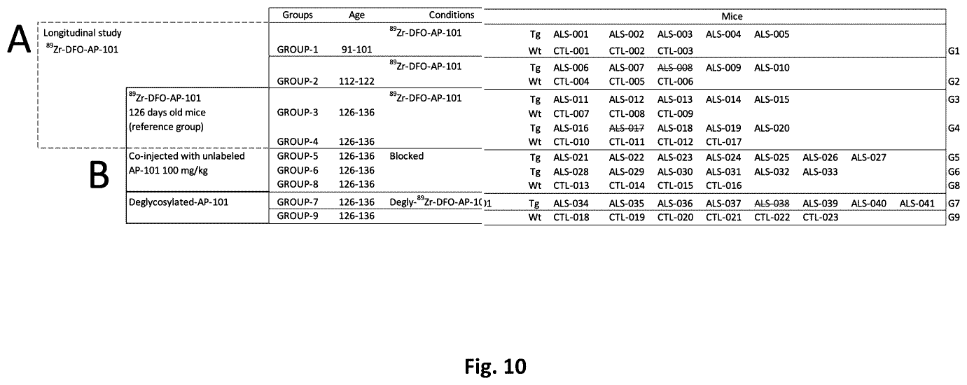

A total of 47 mice (30 Tg and 17 Wt) were used for this study. The distribution of mice in the different experimental groups is presented in FIG. 10 along with the study timeline. The study was completed over several months and used different radiotracer productions. The longitudinal study was set to assess the optimal age and time post administration of ⁸⁹Zr-DFO-AP-101 to perform PET imaging. Tg and Wt mice aged 91, 110, 126 days old, were images at days 1, 4, 7 and 10 post administration of ⁸⁹Zr-DFO-AP-101 (FIG. 10).

From the results obtained, it was decided to set the age for PET imaging to 126 days old and limit PET imaging at days 7 and 10 post administration of ⁸⁹Zr-DFO-AP-101. It is also noteworthy that spots observed at day 7 were for most instance still present at day 10. The method used to analyze PET images was revised to consider the presence of radioactivity spill in onto the spinal cord from the presence of ⁸⁹Zr-DFO-AP-101 within the ventral portion of the vertebra seen in both the Tg and Wt mice.

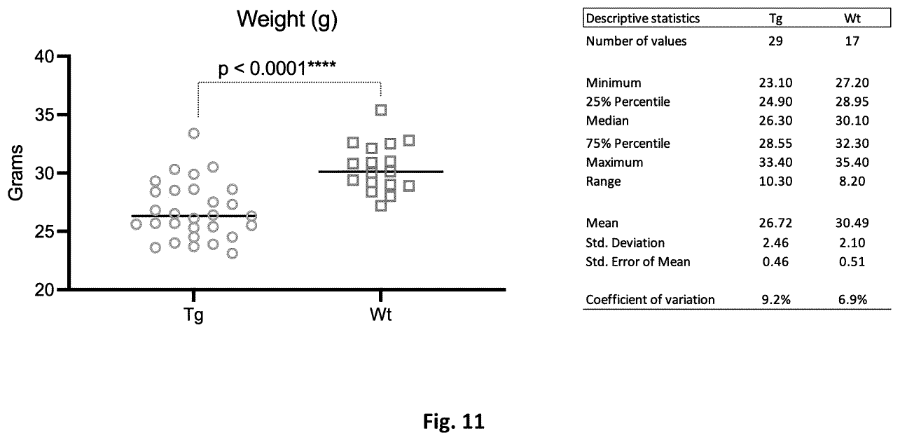

At the time of PET imaging, the Tg mice were smaller as compared to the reference Wt population, which may be explained by their ALS phenotype. Indeed, the weight of the 126 days old Tg mice (25±2 g) was significantly less than that of the Wt mice (30±2 g), FIG. 11.

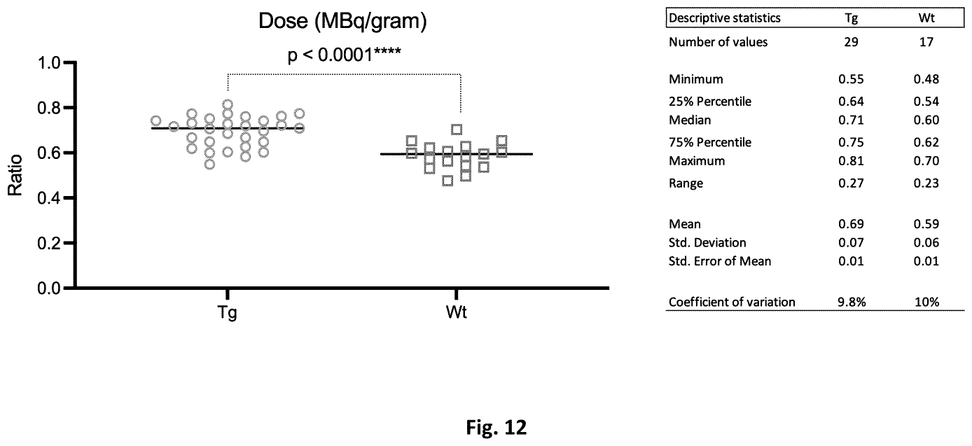

The doses of radiotracer normalized for mice weight (MBq/g) administrated to the Tg mice was significantly higher (0.69±0.07 MBq/g) as compared to that received by the Wt mice (0.59±0.06 MBq/g), FIG. 12. However, the specific activity of ⁸⁹Zr-DFO-AP-101 was high and highly reproducible and all administrated radiotracer doses were sufficient to obtain high quality PET images.

Although PET imaging was performed at day 7 and 10 post administration of the radiotracer the data analysis focused on the results from day 10 since this was the time where the highest uptake value of mSOD1 spots was observed (FIG. 12).

The number of detected spots including false positive spots was more important at day 10 as compared to day−7 for ⁸⁹Zr-DFO-AP-101 (6 spots at day−7 vs 18 spots at day−10,) and for Degly-⁸⁹Zr-DFO-AP-101 (6 spots at day−7 vs 12 spots at day−10). Among these, were some “false positive”, these were either spots occurring in control mice or blocked mice. Except for one mouse, all the false (+) spots observed at day−7 were not seen again at day 10. The distribution of the m-SOD1 spots at day 10 within the mice spinal cord is presented in FIG. 13. All the spots found between the T10 and L2 vertebra, most of them being localized between the thoracic vertebra T11-T13 (⁸⁹Zr-DFO-AP-101: 12/14, 85% vs Degly-⁸⁹Zr-DFO-AP-101: 9/11, 82%).

To assess the specificity of ⁸⁹Zr-DFO-AP-101, a group of mice (13 Tg, 4 Wt) were co-injected with 100 mg/kg of native AP-101 to block the binding of ⁸⁹Zr-DFO-AP-101 to mSOD1. The results are presented in FIGS. 13 and 19. Co-injection with native AP-101 (blocking) considerably reduces the amount of mSOD1 spot detected, which decreased from 67% (6/9) with ⁸⁹Zr-DFO-AP-101 to 15% (5/13) under blocked conditions. The intensity of the spots was also decreased from 8.05±1.30% ID/g to 5.73±0.28 with ⁸⁹Zr-DFO-AP-101 alone or in presence of AP-101 in excess. The ratio to internal reference remained similar for ⁸⁹Zr-DFO-AP-101 (4.62±1.06) and blocked ⁸⁹Zr-DFO-AP-101 (4.70±0.15).

⁸⁹Zr-DFO-AP-101 was found to accumulate in the vertebra ventral bone, which was problematic as the radiation from the vertebra at time would spill into the spinal cord masking potential mSOD1 spots. Degly-⁸⁹Zr-DFO-AP-101 was developed with the intent of reducing the non-specific uptake of the radiotracer in vertebra. The ability of ⁸⁹Zr-DFO-AP-101 and its deglycosylated analog for the detection of mSOD1 spots are compared in FIGS. 13, 14 and 19.

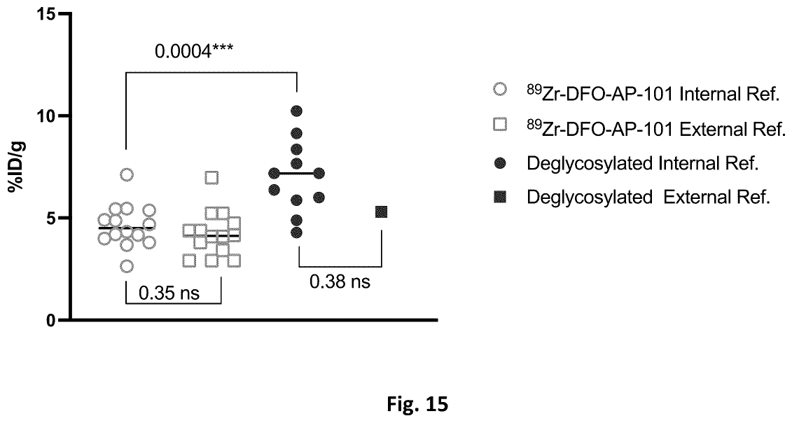

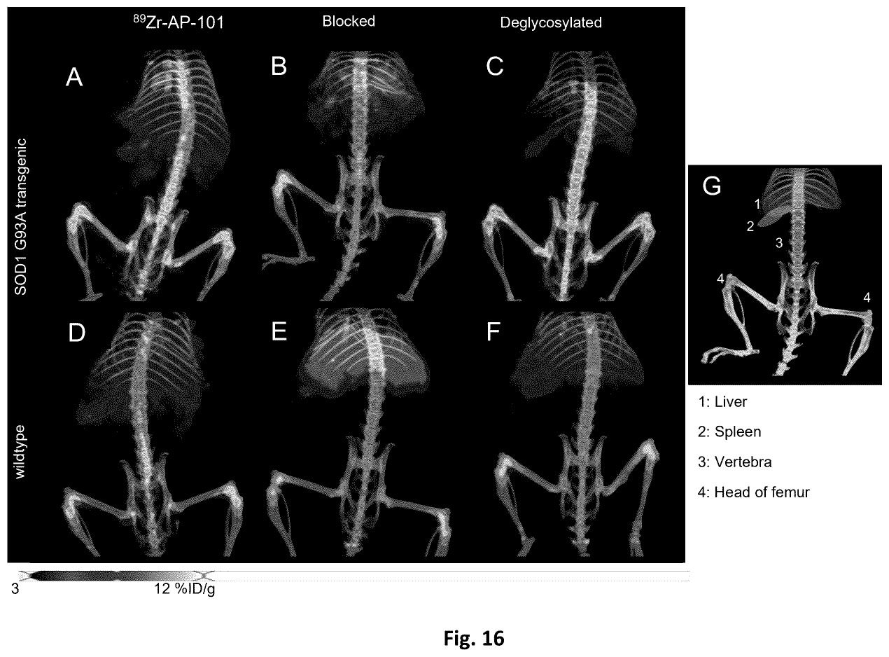

Assuming that all Tg mice had mSOD1 aggregates, deglycosylation increased the efficacy of the radiotracer at detecting mSOD1 aggregates from 67% (6 out of 9 mice) to 100% (7 out of 7 mice), (FIG. 14). Deglycosylation also significantly increased the average intensity of the detected spots from 8.05±1.30% ID/g for ⁸⁹Zr-DFO-AP-101 to 13.14±2.80% ID/g for Degly-⁸⁹Zr-DFO-AP-101 (Two-tails student-t test p=0.01*). However, the spot to internal reference ratio was only slightly higher for Degly-⁸⁹Zr-DFO-AP-101 (1.92±0.34) compared to ⁸⁹Zr-DFO-AP-101 (1.80±0.40) and the difference was not statistically significant (Two-tail Student-t test p=0.08 ns) (FIG. 15). The higher spinal cord background observed for Degly-⁸⁹Zr-DFO-AP-101 was likely due to its increased uptake into the vertebra (FIG. 16).

In summary, ⁸⁹Zr-DFO-AP-101 can specifically engage mSOD1 aggregate as demonstrated by co-injection of blocking quantities of native AP-101. The main limitation being that its accumulation in the ventral vertebra resulted in radioactivity spilling into the spinal cord potentially masking some mSOD1 spots. The presence of radioactivity in the vertebra was attributed to binding to FcγR, which prompted the development of deglycosylated ⁸⁹Zr-DFO-AP-101. Deglycosylation did not reduce the uptake of the radiotracer in the vertebra but resulted in a higher radiotracer uptake into mSOD1 aggregates.

### Example 4: Spinal Cord and Vertebra Uptake of ⁸⁹Zr-DFO-AP-101 and Degly-⁸⁹Zr-DFO-AP-101

The uptake of ⁸⁹Zr-DFO-AP-101 into the spinal cord and vertebra of the Wt an Tg mice was measured and compared for all experimental conditions. An overview of the uptake of ⁸⁹Zr-DFO-AP-101 in the vertebral column of the different groups of mice is given in FIG. 16.

It must be noted that the values measured in the spinal cord, even though a thin ROI was used, may for both the Tg and Wt mice be contaminated by radioactivity coming from the presence of radiotracer within the vertebra bone. Furthermore, for the Tg mice there is a possibility that the ROI might run through mSOD1 aggregates containing radiotracer thus increasing the average % ID/g value. To avoid these problems for some mice we also traced discrete ROI on area devoid of mSOD1 and spilled-in radioactivity.

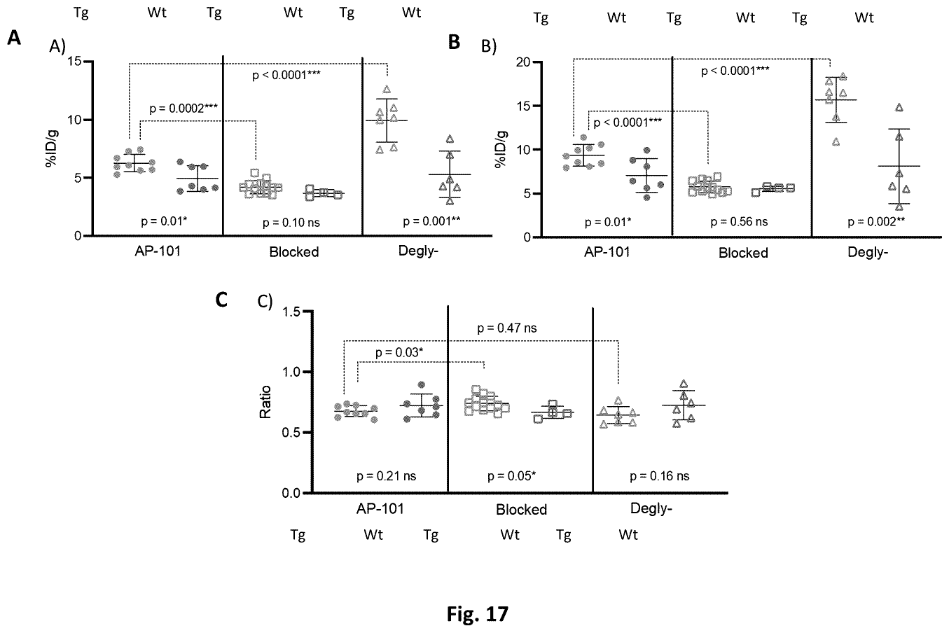

The uptakes of ⁸⁹Zr-DFO-AP-101 and Degly-⁸⁹Zr-DFO-AP-101 in the Tg and Wt mice at day 10 post administration of the radiotracer are compared in FIGS. 3 and 17. The concentration of ⁸⁹Zr-DFO-AP-101 in the spinal cord and vertebra of the Tg mice significantly exceeded that of the Wt mice (p=0.01*), FIG. 17 panels A and B and FIG. 3-2 panels E and F. However, both the Tg and Wt mice shared similar spinal cord to vertebra ventral bone ratio (p=0.21) (FIG. 17 panel C).

Blocking with 100 mg/kg of AP-101, significantly reduced the spinal cord (p=0.0002***) and vertebra (p<0.0001***) uptake of ⁸⁹Zr-DFO-AP-101 FIG. 17 panels A and B. The spinal cord to ventral vertebra bone ratio was also significantly improved by blocking (p=0.03*). In the Wt mice, blocking decreased the uptake of ⁸⁹Zr-DFO-AP-101 in the spinal cord, which was almost statistically significant (p=0.053). However, blocking had no significant effect on either the vertebra uptake of ⁸⁹Zr-DFO-AP-101 (p=0.16) or the spinal cord to vertebra ratio (p=0.31) of Wt mice. Liver uptake was increased in both Tg and Wt mice, but the increase is more important in Wt mice under blocked conditions (FIG. 16).

In the experiments described herein, an accumulation of ⁸⁹Zr-DFO-AP-101 was observed in the anterior portion of vertebra. The accumulation of the radiotracer in the vertebra was at first attributed to the presence of free ⁸⁹Zr, this was later refuted and rather attributed to the binding of the heavy chain glycans of ⁸⁹Zr-DFO-AP-101 to immune cells FcγR (Vivier et al., 2019). This prompted the development and in vivo validation of deglycosylated ⁸⁹Zr-DFO-AP-101 to decrease the in vivo off-target uptake (i.e., reduction of bone, liver and spleen uptakes), to improve target-to-healthy organ contrast and to get higher quality PET images.

In Tg mice, Degly-⁸⁹Zr-DFO-AP-101 showed a significant increase of the uptake of spinal cord (p<0.0001***) and vertebra (p<0.0001***) uptake but did not improve the spinal cord to vertebra ratio (p=47) (FIG. 17 and FIG. 3-2). Lower spinal cord and vertebra backgrounds were seen in Wt mice that received Degly-⁸⁹Zr-DFO-AP-101 resulting in better contrast between Wt and Tg mice. Our findings in Tg mice with Degly-⁸⁹Zr-DFO-AP-101 did little to support our initial hypothesis. Indeed, deglycosylation resulted in a sharp increase uptake of the radiotracer in the vertebra suggesting the presence of mSOD1 in bone or bone marrow and a specific uptake in these tissues.

### Example 5: Head of the Femur and Spleen Uptakes of ⁸⁹Zr-DFO-AP-101 and Degly-⁸⁹Zr-DFO-AP-101

The spleen and the head of the femur were readily visible on the PET images (FIG. 3) and their radiotracer content was found to vary with the experimental conditions. The average % ID/g values measured for ⁸⁹Zr-DFO-AP101 and Degly-⁸⁹Zr-DFO-AP101 within the spleen and the head of the femur are summarized in FIG. 3.

The head of the femur uptake mimicked what was observed for the vertebra bone uptake. Blocking in the Tg mice brought about a significant drop in the head of the femur uptake of ⁸⁹Zr-DFO-AP-101 (p<0.0001***), while a significant increase of this uptake (p=0.0004***) was observed when Degly-⁸⁹Zr-DFO-AP-101 was used for PET imaging (FIG. 3-2 panel G).

Both ⁸⁹Zr-DFO-AP-101 in presence of AP-101 in excess and Degly-⁸⁹Zr-DFO-AP-101 resulted in a statistically significant decrease of the spleen uptake as compared to the uptake of ⁸⁹Zr-DFO-AP-101 (blocking p=0.0003***; deglycosylation p<0.0001***), FIG. 3-2 panel H. One should note that the non-target tissue uptake of the tracers will contribute in increasing their radiation burden the impact of which will have to assess by computing dosimetry.

### Example 6: Biodistribution of ⁸⁹Zr-DFO-AP-101 and Degly-⁸⁹Zr-DFO-AP-101

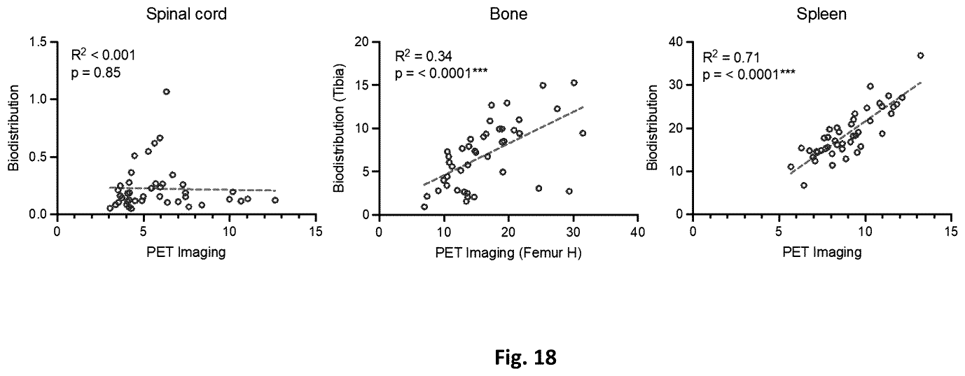

At day 10, after the last PET scan the mice were euthanized, and the tissues of interest removed after mouse perfusion, the residual activity (% ID/g) was measured using a gamma counter. The results for selected tissues are presented in FIG. 18, 19.

For the tissues visible on PET images, the % ID/g measured by PET was correlated with those measured by tissue sampling and counting. This was evaluated for the spinal cord, bone (head of the femur for PET vs tibia from biodistribution) and the spleen (FIG. 18). Except for the spinal cord where the tissue content in radiotracer was low, there was a significant correlation between the % ID/g values measured by PET imaging or biodistribution (bone: R²=0.34, p<0.0001**** and spleen: R²=0.71 p<0.0001****).

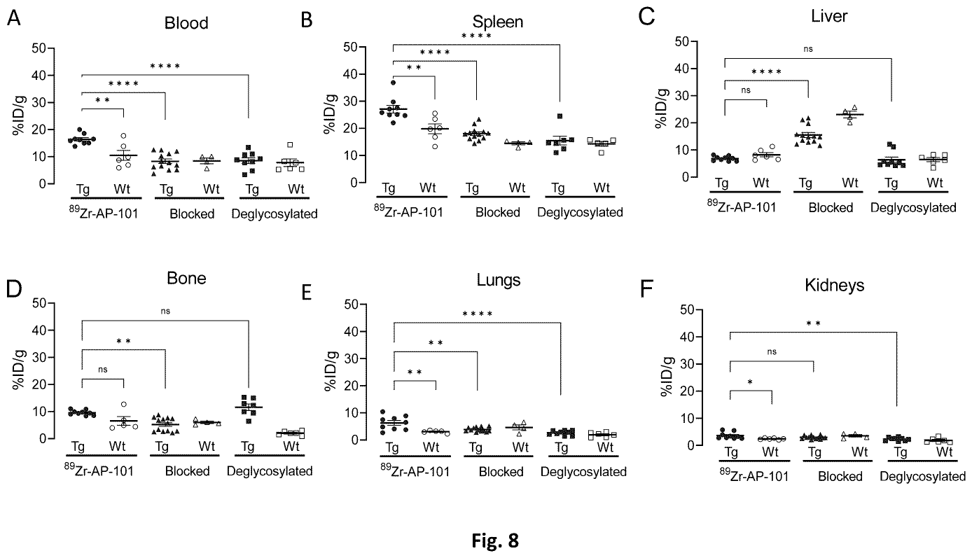

At day−10, following iv administration of ⁸⁹Zr-DFO-AP101 in the Tg mice, the radiotracer was mostly concentrated in the spleen, blood, bone, and liver (FIG. 8). A similar biodistribution pattern was observed for the Wt mice but with a lower radiotracer tissue concentration level. This difference between the Tg and Wt mice is statistically significant for the blood (p=0.05*), lungs (p=0.03*) and muscle (p=0.0007***).

Blocking with AP-101 significantly reduced the uptake of ⁸⁹Zr-DFO-AP-101 in most tissues, blood (p<0.0001***), bone (p=0.002**), spleen (p=0.002**), lungs (p=0.01*) and muscle (p=0.05*), FIG. 8. However, blocking increased the uptake of ⁸⁹Zr-DFO-AP-101 in the liver (p<0.0001***), this was more pronounced in the Wt mice (p=0.0008***). These results are in accordance with PET image analysis (FIG. 16).

Degly-⁸⁹Zr-DFO-AP-101 showed an increase of bone uptake (p=0.09 ns) and significantly decreases in most other tissues such as blood (p=0.0003***), spleen (p=0.0002***), heart (p=0.0008***), lungs (p=0.0001***), kidneys (p=0.01*) and muscle (p=0.002**) when compared to those of ⁸⁹Zr-DFO-AP-101. Deglycosylation of ⁸⁹Zr-DFO-AP-101 had no significant impact on the liver radiotracer concentration.

### Example 7: MisSOD1 Detection in Cartilage Tissue of Bone and Joints of SOD1 G93A Transgenic Mice

Sections of dorsal and ventral vertebrae and from the joint area of the head of femur from SOD1 G93A transgenic and wild type mice have been stained with hematoxylin and with AP-101 and with a commercially available misSOD1 antibody (B8H10 from MEDIMAPS, Gros-Louis et al., J. Neurochem., 113 (2010), 1188-1199) (See FIG. 22). Furthermore, a control staining was performed with 1 nM isotype control antibody. No immunoreactivity could be detected with the isotype control antibody or in wildtype mice. In stainings of the SOD1 G93 transgenic mice misfolded SOD1 was detected with the AP-101 and the B8H10 antibody in both, the bone and joint cross sections.

### Summary of the Experimental Results in Mice

One long-term objective of the experiments described in Examples 1-6 was to validate the feasibility of using an ⁸⁹Zr-based AP-101 radiotracer for PET imaging of mSOD1 aggregates in ALS patients as an example of a tracer for immunoimaging of mSOD1 in vivo in a subject. The preclinical evaluation in Tg and Wt mice showed that it was feasible to use ⁸⁹Zr-DFO-AP-101 to identify discrete mSOD1 aggregates present in Tg mice spinal cord. However, a fair amount of ⁸⁹Zr-DFO-AP-101 was found in vertebra anterior bone portion. Spleen and some vertebra bone uptake was attributed to the binding of the heavy chain glycans of AP-101 to bone marrow T and B lymphocytes FcγR, which prompted the development of a deglycosylated analog of ⁸⁹Zr-DFO-AP-101.

Deglycosylation increased the efficacy of the radiotracer to detect mSOD1 aggregates from 67% to 100%. Both radiotracers were found to be able to engage spinal cord mSOD1 aggregates, the highest uptake and number of detected mSOD1 aggregates being obtained with Degly-⁸⁹Zr-DFO-AP-101. However, deglycosylation also significantly increased the uptake of the radiotracer in vertebra bone and the head of the femur suggesting the presence of mSOD1 in bone and cartilage tissue and a specific uptake in these tissues. Moreover, lower spinal cord and vertebra background was seen in Wt mice that received Degly-⁸⁹Zr-DFO-AP-101 resulting in better contrast between Wt and Tg mice with this ⁸⁹Zr-based AP-101 radiotracer. For these reasons, both radiotracers are suitable, Degly-89Zr-DFO-AP-101 seemed less stable and prone to aggregation, while Degly-⁸⁹Zr-DFO-AP-101 seems to be the most suitable radiotracer for clinical application and has therefore been selected for the human clinical study. The presence of radioactivity in the vertebra will have less impact in humans as compared to mice, because of the differences in spine size and because of higher resolution and better capacity to discriminate lesions by clinical PET/CT.

Furthermore, detection of misfolded SOD1 in cartilage tissue of bones and, joints in two ALS animals models SOD1 G93A and SOD1 G37R transgenic mice indicates its potential role in the decrease of bone health observed in ALS patients and ALS model mice (Caplliure-Llopis et al., (2020) and Zhu et al. (2015) and, in combination with previous findings regarding the potential of antibodies targeting the mutant or misfolded SOD1 in treatment of ALS related symptoms, e.g., extension of the lifespan of treated SOD1 transgenic animals, significant delay in the loss of their body weight and an amelioration of the motor neuron loss (see WO 2012/080518 A1, in particular in Example 9), indicates the potential of treatment with antibodies against pathologically misfolded/aggregated SOD1 species also in the treatment of bone and/or joint related diseases, in particular of diseases showing impairment of bone and/or joint function in connection with presence of misfolded SOD1 in the affected structures.

### Example 8: Dosimetry Estimated from Mouse Biodistribution Data

Safety, tolerability, and pharmacokinetics of AP-101 has been evaluated in two clinical trials. A phase 1 multicenter, open label, single-ascending dose (SAD) study was completed in November 2020 (https://classic.clinicaltrials.gov/ct2/show/NCT03981536). In this study, doses of 100 mg, 500 mg and 2500 mg AP-101 were tested in humans (9 ALS patients initially enrolled). Even the highest of these doses (2500 mg single dose) has not shown events raising clinically significant safety or tolerability concerns, i.e., no serious adverse events (SAEs) and no infusion reactions related to the tested antibody. The observed AEs were related to the procedure of lumbar puncture as such, by which the antibody was administered. The second study is a phase 2a, multicenter, randomized, double-blind, placebo-controlled study (https://classic.clinicaltrials.gov/ct2/show/NCT05039099), initiated in September 2021 with fALS and sALS63 patients (63 participants) and an estimated study completion date of Jul. 30, 2023. So far, no AP-101 related SAEs or toxic effects have been reported from this study as well.

As described herein, the present inventors optimized the ⁸⁹Zr-chelator (DFO-SCN) conjugation on AP-101 lysine residues to obtain a DFO:AP-101 ratio of 1:1. The radiolabeling yield was greater than 97% with an apparent specific activity of 0.656 GBq/mg. ⁸⁹Zr-DFO-AP-101 was shown stable for over 10 days when formulated in PBS, saline or mouse plasma. The feasibility of following the spine uptake of ⁸⁹Zr-DFO-AP-101 by PET/CT imaging was assessed in a limited number of animals described in the foregoing Examples. Three important key steps critical for the overall project were achieved:
⁸⁹Zr-DFO-AP-101 was stable in vitro and in plasma;
the ^{Nat}Zr-DFO-conjugate retained its affinity and specificity for misfolded SOD1; and
⁸⁹Zr-DFO-AP-101 displayed a specific uptake into the spinal cord of murine ALS model (see, e.g., Example 4 and FIGS. 3, 17, 18).

Dosimetry studies performed in C57BL5 mice enabled the estimation of expected injected dose in humans for diagnostic use with PET/CT (see the Aim-3 section at the beginning of the Examples).

In particular, the human radiation dosimetry estimates of ⁸⁹Zr-DFO-AP-101 were calculated from animal biodistribution data obtained by standard method of organ dissection and using the standard MIRD methodology.

In order to estimate the dosimetry of ⁸⁹Zr-DFO-AP-101, a biodistribution experiment was conducted in 126 days old female and male C57BL6 mice. Each mouse was injected through the caudal vein with either ⁸⁹Zr-DFO-AP-101: Population-1 (females n=20, weight 21.6 sdm 1.8 g, dose 0.86 sdm 0.05 MBq, euthanized at 2 h and days 1,2,7,10); Population-2 (Males n=20, weight 33.3 sdm 1.6 g, dose 0.84 sdm 0.04 MBq, euthanized at 2 h and days 1, 2, 7, 10) euthanized at day 10. The data were not corrected for radioactive decay and correspond to the actual tissue radioactivity burden at each time point. The biodistribution data are presented in FIG. 23.

The dosimetry was calculated from radiotracer residence-time using the OLINDA software (OLINDA—Organ Level Internal Dose Assessment Code, Version 2.2.3—copyright Vanderbilt University—2012). The human equivalent organ % ID was estimated from the mice % ID/g values for the organs listed in OLINDA. The time activity curves were fitted to either a mono- or a bi-exponential model using PRISM software (GraphPad Prism version 9.25 for MacOS, GraphPad Software, San Diego, California USA, www.graphpad.com). Residence-times (in hours) were obtained from integration of the fitted time-activity-curves, scaled to the organ mass of the model (mouse or human) chosen for dosimetry calculations. The radiation doses include contribution from beta and gamma rays emitted from ⁸⁹Zr and include contribution from activity within one organ to itself, from neighboring organs and the remainder of the body.

The largest radiation doses were observed in the lungs (0.0535 versus 0.0473 mSv/MBq) and liver (0.0515 versus 0.0336 mSv/MBq) for females versus males, respectively. From the biodistribution results, a high uptake of ⁸⁹Zr-DFO-AP-101 was observed in lungs at 2 h post injection (p.i.), which rapidly decayed over time. However, the uptakes of ⁸⁹Zr-DFO-AP-101 were slightly higher than that in lungs and stable over time in liver, which may contribute to a spill-out activity in lungs form dosimetry calculation. The estimated ⁸⁹Zr-DFO-AP-101 is higher in female (0.237 mSv/MBq) than male (0.171 mSv/MBq) mice (Table 2). It was found that women's effective doses are about 20-40% higher than for men, and this is the case for many radiopharmaceuticals (Stabin MG, 1997). The values are acceptable when compared with other ⁸⁹Zr-based tracers in use in humans. Indeed, the effective dose from the animal data for ⁸⁹Zr-trastuzumab was calculated at 0.44 mSv/MBq in females and 0.39 mSv/MBq in males, which was found to be safe for a phase 0-1 study (Laforest et al. 2016). Based on a few human studies, the estimated radiation dose of ⁸⁹Zr-trastuzumab PET for patients is about 0.54 mSv/MBq. (https://www.ninds.nih.gov/news-events/directors-messages/all-directors-messages/fda-approval-amyotrophic-lateral-sclerosis-als-drug-represents-progress-were-not-done?search-term=amyotrophic%20lateral%20sclerosis%20als%20fact%20sheet.%20Accessed:.%20June%202023; and Laforest et al. 2016.

As can be seen from the preliminary results of the phase I clinical trial study described in Example 10 (design of the study is described in Example 8) these estimates have been found confirmed by the study data with an effective dose of 0.54 mSv/MBq for human female models and an effective dose of 0.48 mSv/MBq ICRP for human male models.

Although this value is higher than that of ¹⁸F-FDG PET, ⁸⁹Zr-based PET tracers are safe for clinical use (Heskamp et al. 2017, Stabin 1997, Laforest et al. 2016, Dijkers et al. 2010, Farr et al. 2021, Yoon et al 2020, Veldhuijzen et al. 2018, Oosting et al. 2016, Even et al 2017).

The toxicity of AP-101 was measured in monkeys. The biologic compound was safe and satisfied the requirements for the clinical trial application. Due to the matching chemical/biological nature, the same is expected from ^{Nat}Zr-DFO-AP-101 and ⁸⁹Zr-DFO-AP-101.

TABLE 2
Extrapolated human organ doses [mSv/MBq] estimates
for ⁸⁹Zr-DFO-AP-101 per unit of administered activity

| | Dose | Dose |
|---|---|---|
| | (mSv/MBq) | (mSv/MBq) |
| Target Organ | FEMALES | MALES |
| Adrenals | 8.27E−03 | 5.67E−03 |
| Brain | 3.32E−04 | 2.97E−04 |
| Breast | 8.46E−03 | |
| Esophagus | 9.89E−03 | 7.03E−03 |
| Eyes | 0.00E+00 | 0.00E+00 |
| Gallbladder Wall | 3.03E−03 | 3.35E−03 |
| Left colon | 5.49E−03 | 5.11E−03 |
| Small Intestine | 2.35E−03 | 1.87E−03 |
| Stomach Wall | 2.24E−02 | 2.02E−02 |
| Right colon | 6.00E−03 | 5.69E−03 |
| Rectum | 1.41E−03 | 5.58E−04 |
| Heart Wall | 3.44E−03 | 3.20E−03 |
| Kidneys | 6.14E−03 | 4.89E−03 |
| Liver | 5.15E−02 | 3.36E−02 |
| Lungs | 5.35E−02 | 4.73E−02 |
| Ovaries | 1.83E−02 | |
| Pancreas | 3.92E−03 | 3.00E−03 |
| Prostate | | 1.06E−04 |
| Salivary Glands | 1.86E−04 | 1.44E−04 |
| Red Marrow | 1.09E−02 | 8.39E−03 |
| Osteogenic Cells | 5.07E−04 | 3.80E−04 |
| Spleen | 7.27E−03 | 7.16E−03 |
| Testes | | 4.26E−03 |
| Thymus | 1.46E−03 | 1.13E−03 |
| Thyroid | 7.96E−03 | 6.93E−03 |
| Urinary Bladder Wall | 1.65E−03 | 5.36E−04 |
| Uterus | 2.32E−03 | |
| Total Body | 0.00E+00 | 0.00E+00 |
| Effective Dose | 2.37E−01 | 1.71E−01 |
| (mSv/MBq) | | |

### Example 9: Generation of ⁸⁹Zr-DFO-AP-101 for PET Imaging in the Clinical Phase 1 Study of Example 10

Antibodies labeled with ⁸⁹Zr are obtained by conjugating ⁸⁹Zr to DFO-AP-101. ⁸⁹Zr can be, e.g. obtained as ⁸⁹Zr-oxalate as described in in the 2. Materials and Methods section at pages 2-6 of Alnahwi et al. (2018) and in an abbreviated manner in Example 1, above. The so obtained ⁸⁹Zr-oxalate was trapped on a quaternary methyl ammonium (QMA) anion exchange column. To automate the process, the subsequent steps can be performed, e.g., on a miniAllinOne (MiniAIO) cassette-based module (Ans, Belgium), also described in Alnahwi et al. (2018), specifically in section 2.4. Automated Cassette-Based Module and Separation Chemistry for ⁸⁹Zr-oxalate.

The production method comprised the following steps. The ⁸⁹Zr-oxalate was transferred (trapped) onto a quaternary methyl ammonium (QMA) anion exchange column. 0.5 mL of a 500 mM NaCl-137 mM HCl solution were then added to the column. The eluate was received from the column in vial. The vial was then transferred to a reactor. The pH of the eluate was adjusted to obtain a pH in the range of 6.5-8.0 with a sodium bicarbonate solution and HEPES buffer solution. About 3 mg of DFO-AP-101 were also added to the pH adjusted eluate. The resulting solution was then warmed to 37° C. for 45 minutes. During the solution was warmed in the reactor PD-10 column (GE Healthcare) was conditioned by rinsing for 3 times with 10 mL 0.9% saline at 1.5 mL/min. After the radiolabeling reaction in the reactor was finished, the solution with the so generated ⁸⁹Zr-DFO-AP-101 was transferred to the conditioned PD-10 column and eluted therefrom with 4 mL of 0.9% saline. The eluate was collected in a new vial and the obtained eluate volume adjusted by adding saline to a final volume of about 10 mL. Thereafter the ⁸⁹Zr-DFO-AP-101 solution was filtered.

The so obtained ⁸⁹Zr-DFO-AP-101 has shown a high radiochemical purity of 90% and above with radiochemical yields greater than about 60%. Radiochemical impurities as ⁸⁸Zr and ⁸⁸Y were at very low level and below 2% within the 29 hours of shelf life after end-of-synthesis (EOS).

To formulate the solution for injections in patients, the solution obtained as described above is formulated in a vehicle comprising again 0.9% saline and 10 mg of gentisic acid. This addition helps preventing radiolysis. The so prepared injection is stable, as indicated above, for 29 hours from EOS. The calculated amount of DFO-AP-101 is about 1.5 mg per injection. The final injection volume will be selected to meet the specified amounts of HEPES or gentisic acid, the amount of which should not exceed 200 μg/injection and 5 mg/injection respectively.

The ⁸⁹Zr-DFO-AP-101 conjugate solution as produced by the method described above has shown the following specifications: appearance—clear and colorless; pH 4.5-8.0; up to 10 mg in the final product; radiochemical identity—R.T.±10% vs STD; radiochemical purity as measured by HPLC of 90% and more; radiochemical purity as measured by instant Thin Layer Chromatography (iTLC) also of 90% and above; radionuclide purity and identification of 98% and above; radionuclide impurities not exceeding 2%. The filter used to purify the product has passed the filter integrity test with above 50 psi. The amount of HEPES buffer was, as indicated above not exceeding 200 μg/injection, gentisic acid not exceeding 5 mg/injection. The obtained ⁸⁹Zr-DFO-AP-101 solution was sterile and its amount of pyrogens was not exceeding 175 EU/vol (17.5 EU/mL). The radionucleic identification half-live was about 4235-5175 minutes (i.e., about 70.58-86.25 hours). Before injection the [⁸⁹Zr]Zr-DFO-AP-101 conjugate solution is checked before injection for clarity

### Example 10: A Single Center, Phase I, Open-Label Study to Evaluate the Biodistribution, Pharmacokinetics and Safety of ⁸⁹Zr-DFO-AP-101 in Healthy Volunteers and ALS Patients

The herein described new clinical trial will be single center, open label study to evaluate biodistribution, pharmacokinetics and safety of ⁸⁹Zr-DFO-AP-101 PET radiotracer in healthy volunteers and patients with ALS.

The objectives and endpoints of the study are summarized in the Table 3 below:

TABLE 3
Objectives and Endpoints of the study

| | Objectives | Endpoints |
|---|---|---|
| Primary | (Part A) To evaluate, by PET | Incidence of adverse events (AEs) and |
| | imaging, the safety, biodistribution | serious adverse events (SAEs) |
| | and dosimetry of ⁸⁹Zr-DFO-AP- | Biodistribution of ⁸⁹Zr-DFO-AP-101 |
| | 101 in healthy men and women | by PET imaging |
| | (Part B) To evaluate, by PET | Dosimetry of ⁸⁹Zr-DFO-AP-101 in |
| | imaging, the safety, biodistribution | human |
| | and dosimetry of ⁸⁹Zr-DFO-AP- | |
| | 101 in ALS (+) patients | |
| Secondary | Evaluation of PK and excretion of | Cmax, AUC and t_{1/2} in plasma over |
| | ⁸⁹Zr-DFO-AP-101 over time | time after injection of ⁸⁹Zr]-DFO-AP- |
| | | 101 |
| | | Excretion measurements of ⁸⁹Zr after |
| | | IV administration |
| Exploratory | Evaluation of differential labeling | Target organ/ tissue ratio in ALS |
| | and uptake of misfolded SOD1 in | patients versus healthy volunteers |
| | motor cortex, brainstem and spinal | Study Design |
| | cord of ALS (+) patients | |

In detail, the following outcomes are to be measured:

### Primary Outcome Measures:

1. Incidence of adverse events (AEs) and serious adverse events (SAEs). The time frame is from day0 (post-injection) to day 14 (end of study). Number of adverse events (AEs) and serious adverse events following administration of ⁸⁹Zr-DFO-AP-101 that are new or worsened (compared to baseline/pre-dose) will be determined.
2. Biodistribution of ⁸⁹Zr-DFO-AP-101. The time frame is here from Pre-dose and at 2 hours, 1, 3, 7, 10 days after injection of the dose. The biodistribution will be assessed by whole-body PET imaging
3. Dosimetry of ⁸⁹Zr-DFO-AP-101 in human. Time frame as in point 2, above. Organ activity concentration (in liver, kidneys, blood, spleen and other organs) will be measured by drawing regions of interest on the PET images.

### Secondary Outcome Measures:

1. Cmax. Time frame is again pre-dose and at 2 hours, 1, 3, 7, 10 days post-dose. Maximal concentration of ⁸⁹Zr-DFO-AP-101 will be measured in plasma overtime after injection
2. Area under the curve (AUC). Time Frame: as in 1. AUC of ⁸⁹Zr-DFO-AP-101 is measured in plasma over time after injection.
3. Residence. Time Frame: as in 1 and 2 above. Time (½) of residence of ⁸⁹Zr-DFO-AP-101 is measured in plasma of the patients.
4. Excretion. Time Frame: as in-3 above. Concentration of ⁸⁹Zr-DFO-AP-101 will be measured in urine of the participants over time.

### Other Pre-Specified Outcome Measures:

1. Differential labeling and uptake. Time Frame again Pre-dose and at 2 hours, 1, 3, 7, 10 days post-dose. Assessment of target organ/tissue ratio in ALS patients will be performed versus healthy volunteers.

### Study Design

### Overall Design

The study is a phase I clinical trial and a 2-part, single center, open label study in healthy volunteers (Part A) and confirmed ALS patients (Part B). The primary goal is evaluating the safety and biodistribution of ⁸⁹Zr-DFO-AP-101 in healthy volunteers and ALS patients.

### Study Visits

Following a screening period of up to 28 days, eligible participants will come to the CRCHUS for all study assessments according to the schedule of events (Table 4). On Day 0, a single intravenous dose of ⁸⁹Zr-DFO-AP-101 40 MBq will be administrated and a 45 min whole body PET/CT acquisition will be performed before the injection and at two hours post injection of the radiotracer. Physical examination, ECG will be performed, vital signs checked and blood/urine samples collected, before and after the injection of the radiotracer (at day 0). Further PET acquisitions will be performed at days 1, 3, 7 and 10 after the injection of the radiotracer. The acquisition time may be adjusted if necessary, but it will not exceed 60 minutes. Participants will be contacted for a final follow-up visit approximately 14 days after study product administration. At each PET/CT scan, a blood and a urine sample (if available) will be collected and their radioactivity content will be measured. Patients with ALS may skip one or two visits if their condition limits multiple visits but should undergo at least 3 scans and the ultimate phone call at 14 days.

### Study Product

The ⁸⁹Zr-DFO-AP-101 is an imaging agent resulting from the combination of radiopharmaceutical and biological compound. The AP-101 is a biological compound acting as a monoclonal antibody specifically recognizing and binding to the human misfolded SOD1. DFO is conjugated to AP-101 and acts as a chelator of zirconium-89 (⁸⁹Zr), a positron emitter (half-life of 3.4 days) that allows uptake detection on PET/CT scan.

### Number of Participants

Twelve patients will be enrolled in total, with 8 healthy volunteers (4 female, 4 male) will be and 4 ALS confirmed patients (+). It is intended to enroll at least one patient with genetic SOD1 mutation and one patient with the sporadic mutation into the group of ALS confirmed patients.

Persons not completing the study will be replaced.

### End of Study Definition

The end of trial occurs following study completion and after the last patient has completed the final study visit and any applicable follow-up. “End of trial” refers to the date of the last visit or last scheduled procedure for the last patient.

### Justification for Dose

The administered dose range for the study (30-40 MBq) is in line with other clinical studies. The amount of protein administrated (less than 2 mg) is significantly lower than the AP-101 therapeutic dose.

### Study Population

### Inclusion Criteria

1. Aged of:
   For healthy participants: Male or female subjects aged 50 years or older
   For ALS patients: Male or female subjects aged 18 years and older
2. Able to remain in a lying position for up to 45 minutes without respiratory support.
3. A) For ALS patients, confirmed diagnostic of definitive ALS according to El-Escorial criteria (Brooks et al., 2000).
   B) for healthy participants: no neurologic condition (confirmed by physical exam)
4. Have venous access sufficient to allow for blood sampling
5. Are reliable and willing to make themselves available for the duration of the study and are willing to follow CRCHUS-specific study procedures.

### Exclusion Criteria

1. Are currently enrolled or were enrolled in the last 12 weeks in any other clinical trial involving a study drug or off-label use of a drug or device, or any other type of medical research judged not to be scientifically or medically compatible with this study.
2. Female participants who are pregnant or breast feeding; or women of childbearing potential (<50 years old) and men who are sexually active who are not willing to use an accepted effective contraceptive method. Nonpregnancy will be confirmed for all female participants<50 years old through a pregnancy test (serum hCG) at screening and at day 0 to the CRCHUS.
   Contraceptive requirements do not apply for participants who are exclusively in same-sex relationships or have nonchildbearing potential (either surgically sterilized [e.g., tubal occlusion, hysterectomy, bilateral salpingectomy] or physiologically incapable of becoming pregnant, or postmenopausal with amenorrhea for at least 12 consecutive months). On the contrary, sexually active participants (female<50 years old or any male with a female partner of child-bearing potential) must use at least one method of contraception, from screening up to 30 days post-injection: Hormonal contraceptive (oral, transdermal patches, vaginal or injectable); Intrauterine device with or without hormones; Condom or vasectomy >30 days prior screening (‘barrier’ method); Diaphragm or cervical cap; Sexual abstinence.
3. Plan to have surgery or other invasive procedure during the course of the study (up to 14 days post-injection)
4. Have a progressive medical illness including, but not limited to, any cardiovascular, hepatic, respiratory, hematological, endocrine, psychiatric or neurological disease, convulsions, or any clinically significant laboratory abnormality at screening and at first visit (DO) that, in the judgment of the medical doctor, indicate a medical problem that would preclude study participation.
5. Have one of these condition (for both patient groups):
   a) hepatic disorder such as hepatic encephalopathy, hepatic laboratory abnormalities (ALT or AST≥3×ULN or total bilirubin≥2×ULN) and hematology abnormalities at screening.
   b) severe chronic kidney disease (eg, an estimated glomerular filtration rate [eGFR]<30 mL/min/1.73m or requires chronic dialysis) at screening.
   c) Have severe active psychiatric illness.
   d) Have a diagnosis of another neurodegenerative disease (e.g. Parkinson disease, Alzheimer's disease, etc).
   e) Have a significant infection or known inflammatory process on screening or at DO.
   f) Alcohol or drug abuse based on patient auto-report
   g) Have a history of relevant atopy or drug hypersensitivity or allergy to antibodies;
   h) Have an abnormal blood pressure (supine) defined as a diastolic blood pressure>90 or <45 mmHg and/or a systolic blood pressure>160 or <90 mmHg. Re-testing may occur once during the screening visit within 2 hours of the initial abnormal blood pressure measurement at the discretion of the investigator.
6. For ALS patients:
   a) Have undergone a tracheostomy for ALS symptoms.
   b) Are on nasal intermittent positive pressure ventilation (NIPPV)>4 h during the day, while awake for the treatment of ALS related symptoms.
   c) Have other causes of neuromuscular weakness.
7. Have received treatment with biologic agents (such as monoclonal antibodies, including marketed drugs and AP-101) within 3 months or 5 half-lives (whichever is longer) prior to study drug injection.
8. Have received any blood or blood products within the 3 months prior to screening.
9. Cannot communicate reliably with the investigator.
10. Are unwilling or unable to give written informed consent.
11. In the opinion of the medical doctor or his/her delegate, are unsuitable for inclusion in the study.

### Screen Failures

Individuals who do not meet the criteria for participation in this study (screen failure) may be re-screened once at the discretion of the investigator with sponsor's approval. If a participant is re-screened, they would be assigned a new participant number and would need to sign a new informed consent form (ICF).

### Intervention

### Product Administration

All participants will receive a single intravenous dose of 40 MBq+10% of ⁸⁹Zr-DFO-AP-101.

The patients will be measured by an appropriate radioactivity calibration system immediately before the injection (see, sections “Study visits”, “Safety Assessment” and Table 4 for details).

### Method of Assignment, Blinding

This is a non-randomized study; eligible participants will be enrolled and receive study product sequentially. This is an open-label study, there is no blinding.

### Dose Modification

Dose levels, sampling schedule, timing of procedures (e.g., time of PK sample collection) may be adjusted in view of emerging safety or PK data during the study. The number of visits for the ALS patients will be scaled down after reviewing the first 3-5 healthy participants. However, the healthy participants will attend all planned visits.

### Concomitant Therapy

In general, concomitant medication will be avoided; however, over-the-counter (OTC) medications may be administered at the discretion of the investigator (e.g., acetaminophen for treatment of headaches, etc.). If the need for concomitant medication (other than OTC medications) arises, inclusion or continuation of the patient will be at the discretion of the investigator after consultation with the sponsor. Any medication used during the course of the study will be documented.

### Study Assessments and Procedures

### Efficacy Assessments

This is a phase I study, so no efficacy assessment will be performed.

### Safety Assessments

According to the CIMS-CRCHUS, the primary safety endpoints are the number of serious and nonserious adverse events. Safety assessment will be conducted at each study visit. No statistical comparisons of both groups (healthy and ALS) will be completed for safety endpoints.

All treatment, medications and study procedure associated to AEs will be listed. Summary statistics for AE and SAE will be provided. Safety assessments may include laboratory tests, vital signs, ECG, physical examination and any auto-reported events. The parameters will be listed and summarized using standard descriptive statistics. Additional analysis will be performed as required.

A safety committee will be constituted in order to assess the causality and management of all AEs, although the medical director might previously have assessed each AE in a timely manner. The Committee will decide whether the event is possibly related to the study, unexpected and serious. and whether the SAE should be reported to the regulatory agencies. If so, the sponsor will submit a full SAE report to the central REB within 7 days, if it is life-threatening or associated with death, or within 15 calendar days for other case scenarios. Consequently, the sponsor will report this possibly related and unexpected SAE to Health Canada and to the Supplier. Development Safety Update Report (DSUR) will also be submitted to the Supplier annually.

The CRCHUS has facilities that will help to ensure participants' safety. It is next to the hospital and to the CHUS emergency, which makes the CRCHUS an excellent place to conduct this phase I. Two neurologists are involved in the study and will be on the safety committee. At least one of them or the medical doctor will do the physical exam for screening and will be on site during the Day 0 (day of injection) of each patient in case of any problem. Medical staff will be at the patient bedside for the duration of the Day 0 (from the injection until 2 hours post-injection). There will be a safety cart near the consultation room in case of any problem. If a patient develops a health condition after his/her participation to the study (up to the last visit), the medical doctor will refer him to a specialized physician to take care of it.

### Dosimetry

Dosimetry will be measured as Laforest et al (2016), previously described. In order to obtain organ activity concentration regions of interest (ROIs) will be drawn on the PET images for the following organs/tissues: liver, kidneys, blood (left ventricular chamber), spleen, muscle, subcutaneous fat and red-marrow. To assess the red marrow activity concentrations, the average activity concentration measured in the lumbar vertebrae will be used. The total activity in each organ will be calculated as the product of the activity concentration and the standard adult organ weight. The decay-corrected data from all patients will be collated to obtain organ time-activity curves. The organ residence times will be derived from the analytical integration of the fit. Using the method of Lemmens et al. (2006), the total patient blood volume will be measured. To assess the remainder of the body residence time, the sum of the residence times observed in the organs will be subtracted from the maximum theoretical residence time. OLINDA/EXM version 2.2.3 software will be used to obtain human dosimetry estimates from the residence times.

### Pharmacokinetics

Blood and urine will be sampled at each PET scan, if available. Blood and urine radioactivity will be measured; remaining sample material will be destroyed after analysis. Pharmacokinetic parameters will be summarized by dose level using descriptive statistics. Mean and individual ⁸⁹Zr-DFO-AP-101 serum concentration-time activity curves will be presented.

### Incidental Findings

Being either healthy or with ALS condition, participants for whom incidental findings are found will receive proper medical follow-up by being referred to their treating physician or a specialist according to the condition. It is possible to observe incidental findings during the PET/CT imaging, through physical examination or through blood/urine laboratory assessment.

### Sample Size Determination

The sample size is not powered on the basis of primary endpoints.

### Statistical Analyses

Statistical analysis of this study will be conducted by the sponsor or its designee. Safety analyses will be conducted for all enrolled patients, whether or not they completed all protocol requirements. For continuous variables, summary statistics will include number of patients, mean, median, standard deviation, minimum, and maximum. Categorical endpoints will be summarized using number of patients, frequency, and percentages.

Preliminary Results from the Phase I Clinical Trial

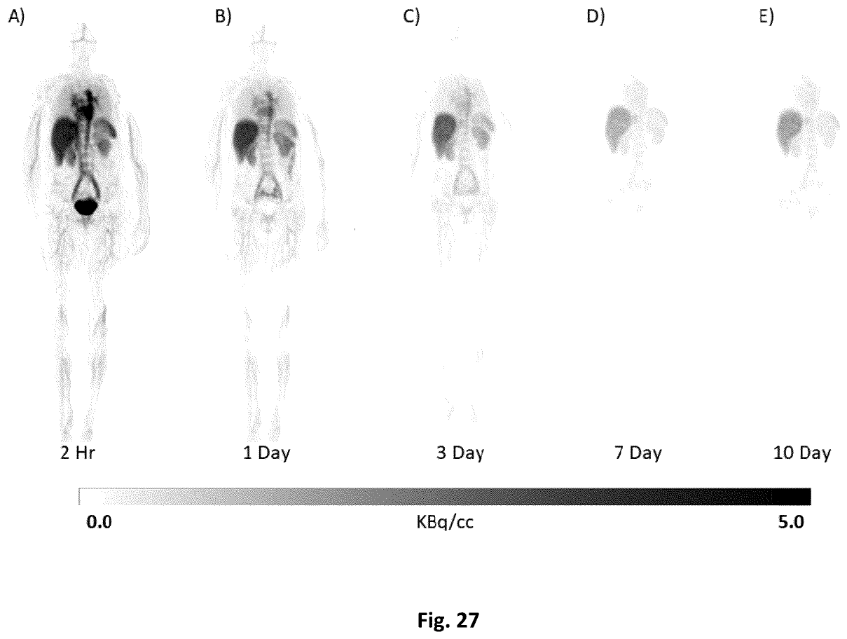

Dosimetry was measured in five healthy controls (3 females and 2 males) as Laforest et al., previously described (Laforest R et al Mol Imaging Biol. 2016; 18(6):952-959). In order to obtain organ activity concentration, regions of interest (ROIs) were drawn on the PET images (see FIG. 27 and its description in the Figures section above) for the following organs/tissues: liver, kidneys, hearth (left ventricular chamber), spleen, bone, bladder.

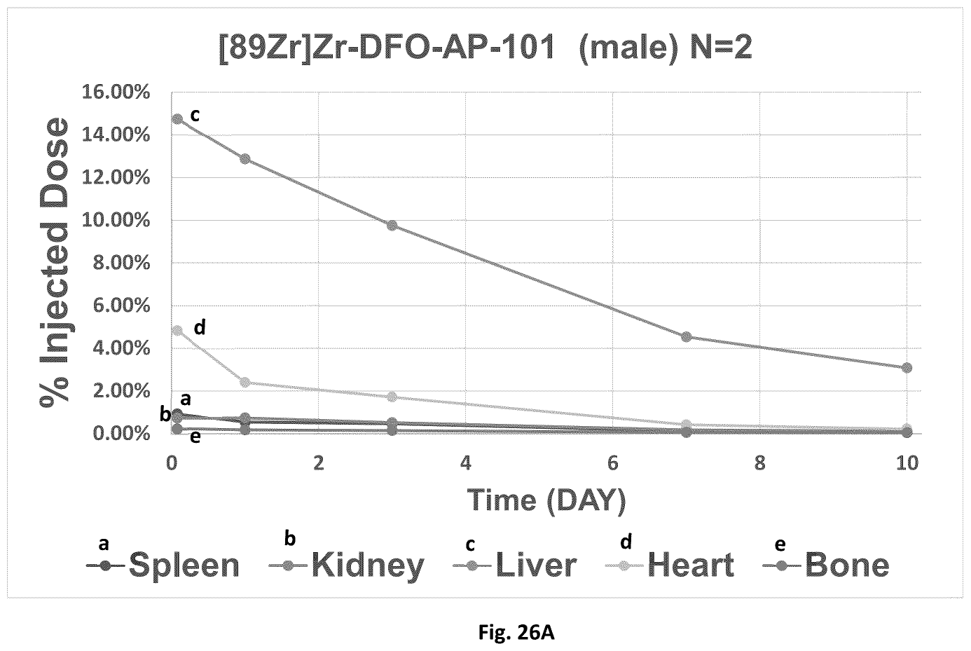

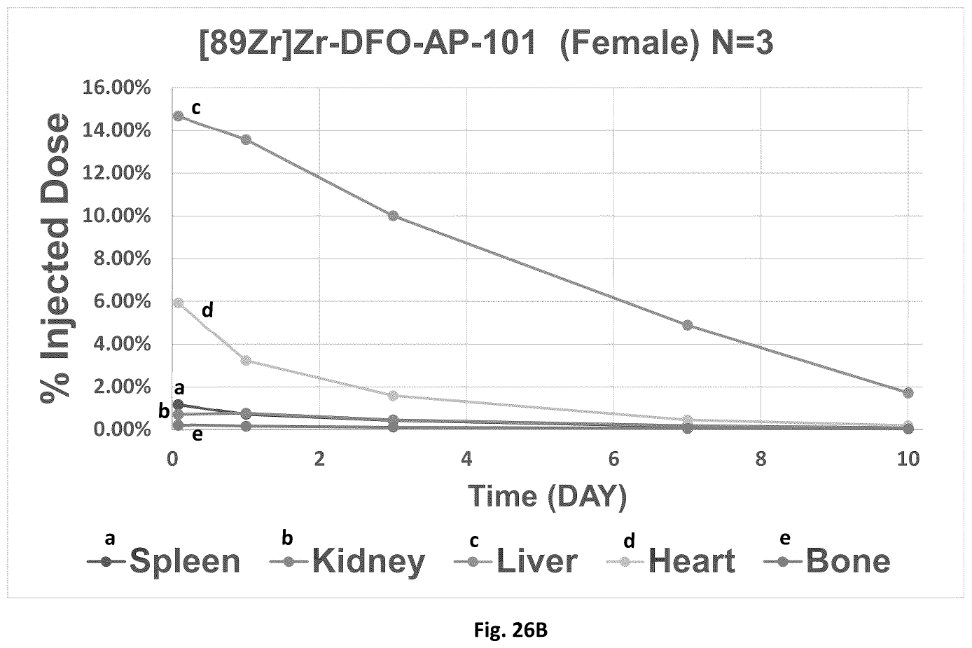

The total activity in each organ was calculated as the product of the activity concentration and the standard adult organ weight. The decay-corrected data was collated from all patients to obtain organ time-activity curves, see FIGS. 26A and 26B. The organ residence times was derived from the analytical integration of the fit. To assess the remainder of the body residence time, the sum of the residence times observed in the organs was subtracted from the maximum theoretical residence time. OLINDA/EXM version 2.2.3 software was used to obtain human dosimetry estimates from the residence times and effective dose to the patient, See Table 6 below.

The dosimetry has been performed as described. Briefly, regions of interest (ROIs) where draw to obtain organ activity concentration on the PET images for different organs/tissues: e.g., liver, kidneys, blood (heart, in particular left ventricular chamber), spleen, bone, bladder. Time percent injected dose curves have been assessed to follow the beavering of the injection of the ⁸⁹Zr-DFO-AP-101. The total activity in each organ is calculated as the product of the activity concentration and the organ weight. The organ residence times is derived from the analytical integration of a fit. To assess the remainder of the body residence time, the sum of the residence times observed in the organs was subtracted from the maximum theoretical residence time.

TABLE 6
Organ radiation doses [mSv/MBq] for ⁸⁹Zr-DFO-AP-101

| | Dose | Dose |
|---|---|---|
| | (mSv/MBq) | (mSv/MBq) |
| Target Organ | FEMALES | MALES |
| Adrenals | 1.05 | 0.96 |
| Brain | 0.27 | 0.25 |
| Breasts | 0.34 | — |
| Esophagus | 0.66 | 0.56 |
| Eyes | 0.27 | 0.25 |
| Gallbladder Wall | 0.86 | 1.16 |
| Left colon | 0.53 | 0.47 |
| Small Intestine | 0.46 | 0.46 |
| Stomach Wall | 0.58 | 0.57 |
| Right colon | 0.54 | 0.57 |
| Rectum | 0.40 | 0.38 |
| Heart Wall | 1.12 | 1.07 |
| Kidneys | 0.92 | 0.89 |
| Liver | 2.65 | 2.24 |
| Lungs | 0.55 | 0.49 |
| Ovaries | 0.41 | |
| Pancreas | 0.84 | 0.64 |
| Prostate | — | 0.38 |
| Salivary Glands | 0.30 | 0.31 |
| Red Marrow | 0.45 | 0.52 |
| Osteogenic Cells | 0.40 | 0.45 |
| Spleen | 1.05 | 0.94 |
| Testes | — | 028 |
| Thymus | 0.51 | 0.49 |
| Thyroid | 0.34 | 0.34 |
| Urinary Bladder Wall | 0.40 | 0.44 |
| Uterus | 0.41 | — |
| Total Body | 0.43 | 0.35 |
| Effective Dose | 0.54 | 0.48 |

Total urinary excretion was 4%, and hepatobiliary excretion was 2%. At 2 h post-injection, 44±5% of the radiotracer remained in the blood pool, with an effective half-life of ±53 h. According to sex-specific dosimetry, the liver is the dose-limiting organ with 2.65 mSv/MBq (female) and 2.24 mSv/MBq (male). The effective dose was 0.54 mSv/MBq (female) and 0.48 mSv/MBq (male).

In summary, as also shown in the above Examples, the labeled-antibody conjugate of the present can be safely used, its clearance is predominantly physical, with an early urinary excretion. Therefore, this tracer can be safely used in patients. Upcoming data in ALS patients will validate its capacity to measure mSOD1 levels in vivo for diagnosis and treatment monitoring.

### REFERENCES

Abou D S, Ku T, Smith-Jones P M. In vivo biodistribution and accumulation of 89Zr in mice. Nucl Med Biol. 2011; 38 (5): 675-681. doi: 10.1016/j.nucmedbio.2010.12.011
Alnahwi A H, Tremblay S, Guérin B. Comparative Study with 89Y-foil and 89Y-pressed Targets for the Production of ⁸⁹Zr. Appl. Sci., 2018, 8, 1579.
Alnahwi A H, Ait-Mohand S, Dumulon-Perreault V, Dory Y L, Guérin B. Promising Performance of 4HMS, a New Zirconium-89 Octadendate Chelator. ACS Omega. 2020; 5 (19): 10731-10739. Published 2020 May 5. doi: 10.1021/acsomega.0c00207
Alnahwi A H, Tremblay S, Ait-Mohand S, Beaudoin J F, Guérin B. Automated radiosynthesis of 68Ga for large-scale routine production using 68Zn pressed target. Appl Radiat Isot. 2020; 156:109014. doi: 10.1016/j.apradiso.2019.109014
[ALSA] ALS Association resource page. Available at: http://www.alsa.org/about-als/facts-you-should-know.html.
Ayers J I, Fromholt S E, O'Neal V M, Diamond J H, Borchelt D R. Prion-like propagation of mutant SOD1 misfolding and motor neuron disease spread along neuroanatomical pathways. Acta Neuropathol. 2016; 131 (1): 103-114. doi: 10.1007/s00401-015-1514-0
Bensch F, Lamberts L E, Smeenk M M, et al. ⁸⁹Zr-Lumretuzumab PET Imaging before and during HER3 Antibody Lumretuzumab Treatment in Patients with Solid Tumors. Clin Cancer Res. 2017; 23 (20): 6128-6137. doi: 10.1158/1078-0432.CCR-17-0311
Bidhendi E E, Bergh J, Zetterström P, Andersen P M, Marklund S L, Brännström T. Two superoxide dismutase prion strains transmit amyotrophic lateral sclerosis-like disease. J Clin Invest. 2016; 126 (6): 2249-2253. doi: 10.1172/JCI84360
Bosco D A, Landers J E. Genetic determinants of amyotrophic lateral sclerosis as therapeutic targets. CNS Neurol Disord Drug Targets. 2010; 9 (6): 779-790. doi: 10.2174/187152710793237494
Brooks B R, Miller R G, Swash M, Munsat T L, World Federation of Neurology Research Group on Motor Neuron D. El Escorial revisited: revised criteria for the diagnosis of amyotrophic lateral sclerosis. Amyotroph Lateral Scler Other Motor Neuron Disord. 2000; 1 (5): 293-299.
Bruijnen, S.; Tsang, A. S. M.; Raterman, H.; Ramwadhdoebe, T.; Vugts, D.; van Dongen, G.; Huisman, M.; Hoekstra, O.; Tak, P. P.; Voskuyl, A.; et al. B-cell imaging with zirconium-89 labelled rituximab PET-C T at baseline is associated with therapeutic response 24 weeks after initiation of rituximab treatment in rheumatoid arthritis patients. Arthritis Res. Ther. 18, 266 (2016).
Caplliure-Llopis J, Escrivá D, Benlloch M, de la Rubia Ortí J E, Estrela J M, Barrios C., Poor Bone Quality in Patients With Amyotrophic Lateral Sclerosis., Front. Neurol. 11:599216. doi: 10.3389/fneur.2020.599216 Eckelman W C, Kilbourn M R, Joyal J L, Labiris R, Valliant J F. Justifying the number of animals for each experiment. Nucl Med Biol. 2007; 34 (3): 229-232. doi: 10.1016/j.nucmedbio.2007.01.005
Dijkers, E. C.; Oude Munnink, T. H.; Kosterink, J. G.; Brouwers, A. H.; Jager, P. L.; de Jong, J. R.; van Dongen, G. A.; Schroder, C. P.; Lub-de Hooge, M. N.; de Vries, E. G. Biodistribution of ⁸⁹Zr-trastuzumab and PET imaging of HER2-positive lesions in patients with metastatic breast cancer. Clin. Pharmacol. Ther. 87, 586-592 (2010).
Eckelman W C. Further discussions on choosing the number of animals for an experiment. Nucl Med Biol. 2008; 35 (1): 1-2. doi: 10.1016/j.nucmedbio.2007.10.002
Forsberg K, Jonsson P A, Andersen P M, et al. Novel antibodies reveal inclusions containing non-native SOD1 in sporadic ALS patients. PLoS One. 2010; 5 (7): e11552. Published 2010 Jul. 14. doi: 10.1371/journal.pone.0011552
Farr A C, Xiong M P. Challenges and Opportunities of Deferoxamine Delivery for Treatment of Alzheimer's Disease, Parkinson's Disease, and Intracerebral Hemorrhage. Mol Pharm. 1; 18 (2): 593-609 (2021). doi: 10.1021/acs.molpharmaceut.0c00474. Epub 2020 Oct. 9. PMID: 32926630; PMCID: PMC8819678.
Even, A. J.; Hamming-Vrieze, O.; van Elmpt, W.; Winnepenninckx, V. J.; Heukelom, J.; Tesselaar, M. E.; Vogel, W. V.; Hoeben, A.; Zegers, C. M.; Vugts, D. J.; et al. Quantitative assessment of Zirconium-89 labeled cetuximab using PET/C T imaging in patients with advanced head and neck cancer: A theragnostic approach. Oncotarget 8, 3870-3880 (2017).
Forsberg K, Andersen P M, Marklund S L, Brännström T. Glial nuclear aggregates of superoxide dismutase-1 are regularly present in patients with amyotrophic lateral sclerosis. Acta Neuropathol. 2011; 121 (5): 623-634. doi: 10.1007/s00401-011-0805-3
Gaudin E, Thibaudeau C, Arpin L, Leroux J D, Toussaint M, Beaudoin J F, Cadorette J, Paillé M, Pepin C M, Koua K, Bouchard J, Viscogliosi N, Paulin C, Fontaine R, Lecomte R. Performance evaluation of the mouse version of the LabPET II PET scanner. Phys. Med. Biol. 2021; 66:065019.
Grad L I, Cashman N R. Prion-like activity of Cu/Zn superoxide dismutase: implications for amyotrophic lateral sclerosis. Prion. 2014; 8 (1): 33-41. doi: 10.4161/pri.27602
Graffmo K S, Forsberg K, Bergh J, et al. Expression of wild-type human superoxide dismutase-1 in mice causes amyotrophic lateral sclerosis. Hum Mol Genet. 2013; 22 (1): 51-60. doi: 10.1093/hmg/dds399
https://www.ninds.nih.gov/news-events/directors-messages/all-directors-messages/fda-approval-amyotrophic-lateral-sclerosis-als-drug-represents-progress-were-not-done?search-term=amyotrophic%20lateral%20sclerosis%20als%20fact%20sheet.%20Accessed:%20June%202023
He, F. (2011). BCA (Bicinchoninic Acid) Protein Assay. Bio-101: e44. DOI: 10.21769/BioProtoc.44.
Heskamp S, Raavé R, Boerman O, Rijpkema M, Goncalves V, Denat F. ⁸⁹Zr-Immuno-Positron Emission Tomography in Oncology: State-of-the-Art ⁸⁹Zr Radiochemistry. Bioconjug Chem. 2017; 28 (9): 2211-2223. doi: 10.1021/acs.bioconjchem.7b00325
Holland J P, Divilov V, Bander N H, Smith-Jones P M, Larson S M, Lewis J S. ⁸⁹Zr-DFO-J591 for immunoPET of prostate-specific membrane antigen expression in vivo. J Nucl Med. 2010; 51 (8): 1293-1300. doi: 10.2967/jnumed.110.076174
Joshi R, Gangabhagirathi R, Venu S, Adhikari S, Mukherjee T. Antioxidant activity and free radical scavenging reactions of gentisic acid: in-vitro and pulse radiolysis studies. Free Radic Res. 2012; 46 (1): 11-20. doi: 10.3109/10715762.2011.633518.
Jauw, Y. W.; Zijlstra, J. M.; de Jong, D.; Vugts, D. J.; Zweegman, S.; Hoekstra, O. S.; van Dongen, G. A.; Huisman, M. C. Performance of ⁸⁹Zr-Labeled-Rituximab-PET as an Imaging Biomarker to Assess CD20 Targeting: A Pilot Study in Patients with Relapsed/Refractory Di_use Large B Cell Lymphoma. PLoS ONE 12, e0169828 (2017).
   Laforest R, Lapi S E, Oyama R, Bose R, Tabchy A, Marquez-Nostra B V, Burkemper J, Wright B D, Frye J, Frye S, Siegel B A, Dehdashti F. [⁸⁹Zr] Trastuzumab: Evaluation of Radiation Dosimetry, Safety, and Optimal Imaging Parameters in Women with HER2-Positive Breast Cancer. Mol Imaging Biol. 2016 December; 18 (6): 952-959. doi: 10.1007/s11307-016-0951-z. PMID: 27146421; PMCID: PMC5096950.
Lemmens H J, Bernstein D P, Brodsky J B. Estimating blood volumein obese and morbidly obese patients. Obes Surg 2006; 16:773-6.
Maier M, Welt T, Wirth F, et al. A human-derived antibody targets misfolded SOD1 and ameliorates motor symptoms in mouse models of amyotrophic lateral sclerosis. Sci Transl Med. 2018; 10 (470): eaah3924. doi: 10.1126/scitranslmed.aah3924
Makris, N. E.; Boellaard, R.; van Lingen, A.; Lammertsma, A. A.; van Dongen, G. A.; Verheul, H. M.; Menke, C. W.; Huisman, M. C. PET/C T-derived whole-body and bone marrow dosimetry of ⁸⁹Zr-cetuximab. J. Nucl. Med. 56, 249-254 (2015).
Menke-van der Houven van Oordt C W, McGeoch A, Bergstrom M, et al. Immuno-PET Imaging to Assess Target Engagement: Experience from ⁸⁹Zr-Anti-HER3 mAb (GSK2849330) in Patients with Solid Tumors. J Nucl Med. 2019; 60 (7): 902-909. doi: 10.2967/jnumed.118.214726
Oosting, S. F.; van Asselt, S. J.; Brouwers, A. H.; Bongaerts, A. H.; Steinberg, J. D.; de Jong, J. R.; Lub-deHooge, M. N.; van derHorst-Schrivers, A. N.; Walenkamp, A. M.; Hoving, E. W.; et al. ⁸⁹Zr-Bevacizumab PET Visualizes Disease Manifestations in Patients with von Hippel-Lindau Disease. J. Nucl. Med. 57, 1244-1250 (2016).
Pandya D N, Bhatt N, Yuan H, Day C S, Ehrmann B M, Wright M, Bierbach U, Wadas T J. Zirconium tetraazamacrocycle complexes display extraordinary stability and provide a new strategy for zirconium-89-based radiopharmaceutical development. Chem. Sci. 2017; 8:2309-2314.
Paré B, Lehmann M, Beaudin M, et al. Misfolded SOD1 pathology in sporadic Amyotrophic Lateral Sclerosis. Sci Rep. 2018; 8 (1): 14223. Published 2018 Sep. 21. doi: 10.1038/s41598-018-31773-z
Stabin M G. Health concerns related to radiation exposure of the female nuclear medicine patient. Environ Health Perspect. 105 (Suppl 6): 1403-1409 (1997).
Tateno F, Sakakibara R, Kawashima K, et al. Amyotrophic lateral sclerosis presenting respiratory failure as the sole initial manifestation. Case Rep Neurol. 2014; 6 (2): 213-216. Published 2014 Aug. 14. doi: 10.1159/000366191
Tokuda E, Takei Y I, Ohara S, Fujiwara N, Hozumi I, Furukawa Y. Wild-type Cu/Zn-superoxide dismutase is misfolded in cerebrospinal fluid of sporadic amyotrophic lateral sclerosis. Mol Neurodegener. 2019; 14 (1): 42. doi: 10.1186/s13024-019-0341-5
Torres M L, Wanionok N E, McCarthy A D, Morel G R, Fernandez J M, Systemic oxidative stress in old rats is associated with both osteoporosis and cognitive impairment, Experimental Gerontology, 2021; 156 https://doi.org/10.1016/j.exger.2021.111596
Veldhuijzen van Zanten, S. E. M.; Sewing, A. C. P.; van Lingen, A.; Hoekstra, O. S.; Wesseling, P.; Meel, M. H.; van Vuurden, D. G.; Kaspers, G. J. L.; Hulleman, E.; Bugiani, M. Multiregional Tumor Drug-Uptake Imaging by PET and Microvascular Morphology in End-Stage Di_use Intrinsic Pontine Glioma. J. Nucl. Med. 59, 612-615 (2018).
Vivier D, Sharma S K, Adumeau P, Rodriguez C, Fung K, Zeglis B M. The Impact of FcγRI Binding on Immuno-PET. J Nucl Med. 2019; 60:1174-1182. doi: 10.2967/jnumed. 118.223636
Vu L T, Bowser R. Fluid-Based Biomarkers for Amyotrophic Lateral Sclerosis. Neurotherapeutics. 2017; 14 (1): 119-134. doi: 10.1007/s13311-016-0503-x
Wang, Z., Zhu, J. & Lu, H. Antibody glycosylation: impact on antibody drug characteristics and quality control. Appl Microbiol Biotechnol 104, 1905-1914 (2020). https://doi.org/10.1007/s00253-020-10368-7
Wang H, Yi J, Li X, Xiao Y, Dhakal K, Zhou J. 2018. ALS-associated mutation SOD1 (G93A) leads to abnormal mitochondrial dynamics in osteocytes. Bone (2017); 106:126-138. doi: 10.1016/j.bone.2017.10.010.
Yoon J K, Park B N, Ryu E K, An Y S, Lee S J. Current Perspectives on ⁸⁹Zr-PET Imaging. Int. J. Mol. Sci. 21, 4309 (2020); doi: 10.3390/ijms21124309
Zhu K, Yi J, Xiao Y, Lai Y, Song P, Zheng W, Jiao H, Fan J, Wu C, Chen D, Zhou J, Xiao G., Impaired Bone Homeostasis in Amyotrophic Lateral Sclerosis Mice with Muscle Atrophy, J Biol Chem. 2015 Mar. 27; 290 (13): 8081-94. doi: 10.1074/jbc.M114.603985. Epub 2015 Feb. 3.

1. A labeled-antibody conjugate for use in a method of diagnosing amyotrophic lateral sclerosis (ALS) or monitoring drug therapy and/or progression of ALS in a subject, wherein the method comprises in vivo immunoimaging of misfolded SOD1 in a subject and the labeled-antibody conjugate comprises:
(a) an antibody that specifically binds to misfolded SOD1; and
(b) a detection label conjugated to the antibody.

2. The labeled-antibody conjugate for use according to claim 1, wherein the detection label is a radionuclide.

3. The labeled-antibody conjugate for use according to claim 2, wherein the radionuclide is ⁸⁹Zr.

4. The labeled-antibody conjugate for use according to any one of the preceding claims, wherein the detection label is conjugated to the antibody with a bifunctional chelator or prosthetic group, preferably wherein the bifunctional chelator or prosthetic group is conjugated randomly on lysine residues of the antibody.

5. The labeled-antibody conjugate for use according to claim 4, wherein the bifunctional chelator comprises p-SCN-Bn desferoxamine (SCN-Bn-DFO).

6. The labeled-antibody conjugate for use according to any one of the preceding claims, wherein the antibody is characterized by comprising in its variable region, i.e., binding domain the six CDRs of the variable heavy (VH) and variable light (VL) chain, wherein:
(a) VH-CDR1 comprises the amino acid sequence of SEQ ID NO: 3 or a variant thereof, wherein the variant comprises one or two amino acid substitutions,
(b) VH-CDR2 comprises the amino acid sequence of SEQ ID NO: 4 or a variant thereof, wherein the variant comprises one or two amino acid substitutions,
(c) VH-CDR3 comprises the amino acid sequence of SEQ ID NO: 5 or a variant thereof, wherein the variant comprises one or two amino acid substitutions,
(d) VL-CDR1 comprises the amino acid sequence of SEQ ID NO: 8 or a variant thereof, wherein the variant comprises one or two amino acid substitutions,
(e) VL-CDR2 comprises the amino acid sequence of SEQ ID NO: 9 or a variant thereof, wherein the variant comprises one or two amino acid substitutions, and
(f) VL-CDR3 comprises the amino acid sequence of SEQ ID NO: 10 or a variant thereof, wherein the variant comprises one or two amino acid substitutions.

7. The labeled-antibody conjugate for use according to any one of the preceding claims, wherein the antibody is characterized by comprising in its variable region, i.e., binding domain a VH chain and a VL chain, wherein
(a) the VH chain comprises the amino acid sequence depicted in SEQ ID NO: 1 or 2 or a variant thereof, wherein the variant comprises one or more amino acid substitutions; and
(b) the VL chain comprises the amino acid sequence depicted in SEQ ID NO: 6 or 7, or a variant thereof, wherein the variant comprises one or more amino acid substitutions;
preferably wherein the VH and VL chain amino acid sequence is at least 90% identical to SEQ ID NO: 1 or 2 and 6 or 7, respectively.

8. The labeled antibody conjugate for use according to any of the preceding claims, wherein the antibody comprises
two heavy chains, wherein each heavy chain comprises the amino acid sequence depicted in SEQ ID NO: 14; and
two light chains, wherein each light chain each light chain comprises the amino acid sequence depicted in SEQ ID NO: 15.

9. The labeled-antibody conjugate for use according to any one of the preceding claims, wherein the antibody is AP-101.

10. The labeled-antibody conjugate for use according to any one of the preceding claims, wherein the antibody is glycosylated or deglycosylated.

11. The labeled-antibody conjugate for use according to any one of the preceding claims, wherein the imaging comprises positron emission tomography (PET) imaging, single photon emission computed tomography (SPECT) imaging, or combinations thereof with computed tomography (CT).

12. A drug for use in the treatment of ALS in a subject which (i) has been diagnosed for suffering or developing ALS and/or (ii) is monitored for efficacy of drug therapy and/or disease progression of ALS by a method as defined in any one of the preceding claims, preferably wherein the drug is selected from Riluzole (Rilutek®), Edaravone (Radicava®), Tofersen (BIIB067), AP-101, dextromethorphan HBr and quinidine sulfate (Nuedexta®), Reldesemtiv (CY5031), Arimoclomol, Levosimendan, Fasudil, pyrimethamine, (Daraprim™), rapamycin, baclofen (Gablofen®, Kemstro®, Lioresal®), diazepam (Diastat®, Valium®), amitriptyline (Elavil®), trihexyphenidyl, Scopoderm® (scopolamine patch), glycopyrrolate (Robinul®), Ravulizumab (BNJ441, ALXN1210, or Ultomiris®) and Eculizumab (Soliris®).

13. A labeled-antibody conjugate as defined in any one of the preceding claims.

14. The labeled-antibody conjugate of claim 13, wherein the labeled-antibody conjugate is suitable for in vivo immunoimaging of misfolded SOD1 in a subject.

15. The labeled-antibody conjugate of claim 13 or 14, wherein the antibody conjugate is ⁸⁹Zr-DFO-AP-101 and comprises
(a) AP-101 as the antibody that specifically binds to misfolded SOD1;
(b) p-SCN-Bn desferoxamine (SCN-Bn-DFO) as the bifunctional chelator, and
(c)⁸⁹Zr as the radionuclide.

16. An antibody that specifically binds to misfolded SOD1, which comprises a bifunctional chelator, preferably wherein the bifunctional chelator is p-SCN-Bn desferoxamine (SCN-Bn-DFO).

17. An imaging composition comprising the labeled-antibody conjugate of any one of claims 13 to 15, preferably further comprising NaCl and gentisic acid, at a pH 4.5-8.0.

18. The imaging composition of claim 17, which is a 10 mL solution constituted of NaCl (0.9%) and gentisic acid (10 mg) in a sealed vial, containing about 10 mg labeled-antibody conjugate and between about 300 and 500 MBq, preferably wherein the amount of labeled-antibody conjugate vs. unlabeled antibody is greater than 97% and/or wherein the labeled-antibody conjugate has an apparent specific activity of about 0.656 GBq/mg, optionally wherein the injected solution can be diluted in NaCl prior injection.

19. A diagnostic composition or kit comprising
the labeled antibody conjugate of any one of claims 13 to 15;
the imaging composition of claim 17 or 18; or
the antibody of claim 16, optionally further comprising a detection label, preferably comprising a radionuclide, preferably wherein the radionuclide is ⁸⁹Zr;
and optionally instructions for use in a method as defined in any one of claims 1 to 11.

20. A method of in vivo immunoimaging of superoxide dismutase (SOD1) useful as a marker of amyotrophic lateral sclerosis (ALS) or as a marker for efficacy of drug therapy of ALS in subject, comprising
(a) administering a labeled-antibody conjugate of any one of claims 13 to 15 to a subject; and
(b) detecting the presence of the labeled-antibody conjugate in the subject in vivo by imaging.

21. The method of claim 20, comprising conducting a radiographic imaging method on the subject after administration of the labeled-antibody conjugate; and making a radiographic image of the subject for detecting the labeled-antibody conjugate; wherein the radiographic image is diagnostic for the presence of misfolded and/or aggregated SOD1 in the subject.

22. A method of generating an image of a human body, preferably the human body of an ALS patient comprising administering a labeled-antibody conjugate of any one of claims 13 to 15 to the human body and generating and image of at least a part of the human body to which the labeled-antibody conjugate has distributed.

23. The method of any one of claims 20 to 22, wherein the labeled-antibody conjugate is present in or provided by the imaging composition of claim 17 or 18.

24. The method of any one of claims 20 to 23 comprising contacting cells, tissue or an organ with the labeled antibody conjugate, wherein the labeled antibody conjugate is administered with a target dose in the range of 10-100 MBq; and making a radiographic image for detecting the labeled antibody conjugate.

25. The method of claim 24, wherein the labeled antibody conjugate is administered with a target dose in the range of about 40 MBq±10%.

26. The method of any one of claims 20 to 25, wherein the labeled antibody conjugate is administered with a target dose which is significantly lower than the therapeutic dose of the antibody.

27. The method of claim 26, wherein the labeled antibody conjugate is administered with a target dose, wherein the antibody protein amount is less than 2 mg.

28. The method of any one of claims 20 to 27, wherein the labeled-antibody conjugate is administered via intravenous injection.

29. The method of claim 28, wherein the final volume for injection will be based on the HEPES or gentisic acid contents for which the specifications are ≤200 μg/injection and ≤5 mg/injection respectively.

30. The method of any one of claims 20 to 29, wherein the labeled-antibody conjugate is administered at a single dose.

31. The method of any one of claims 20 to 30, wherein the imaging method is performed at a time after administration of the labeled-antibody conjugate, wherein the time is selected from the group consisting of 1 hour, 2 hours, 3 hours, and 24 hours.

32. The method of claim 31, wherein the imaging method is performed 2 hours post injection.

33. The method of claim 31 or 32, wherein the imaging method is further performed 1, 3, 7 and/or 10 days post injection.

34. The method of any one of claims 20 to 33, wherein the imaging method is selected from the group consisting of planar imaging, positron emission tomography (PET), single photon computed tomography (SPECT) and combinations thereof with computed tomography (CT).

35. The method of any one of claims 20 to 34, wherein the imaging is performed for a region of interest (ROI) selected from the group of organs/tissues consisting of liver, kidney, blood (left ventricular chamber), spleen, muscle, subcutaneous fat, red-marrow, bone, joint, cartilage tissue.

36. The method of any one of claims 20 to 35, wherein the labeled-antibody conjugate is administered to predict effectiveness of a therapy drug for treating a disease caused by and/or associated with the presence of misfolded and/or aggregated SOD1.

37. The method of claim 36, wherein the therapy drug is selected from a drug as defined in claim 9.

38. The method of any one of claims 20 to 37, wherein the labeled-antibody conjugate is administered to diagnose ALS or monitor the effect of the therapeutic treatment of ALS, the method comprising conducting a radiographic imaging method on the subject after administration of the labeled-antibody conjugate; and making a radiographic image of the subject for detecting the labeled-antibody conjugate; wherein the radiographic image is diagnostic for ALS and the effectiveness of the therapeutic treatment of ALS, respectively.

39. An anti-SOD1 antibody or equivalent SOD1 binding molecule for use in targeting misfolded and/or aggregated SOD1 in bone and/or joints of a subject, preferably wherein the antibody is the AP-101 antibody.

40. The anti-SOD1 antibody or equivalent SOD1 binding molecule for use according to claim 39, wherein the anti-SOD1 antibody or equivalent SOD1 binding molecule is a labeled antibody conjugate as defined in any one of the preceding claims.

41. ⁸⁹Zr or ⁸⁹Zr-DFO for use in immunoimaging of misfolded and/or aggregated SOD1 in a subject, preferably for use in the labeled antibody conjugate or method of any one of the preceding claims and/or for use in early diagnostic of ALS and/or in the monitoring of therapeutic interventions of ALS.

42. Use of the antibody or the labeled-antibody conjugate of any one of claims 13 to 16 for the manufacture of an imaging composition for use in the method of any one of claims 20 to 38.

43. Method for preparation of a solution comprising the ⁸⁹Zr-DFO-AP-101 antibody conjugate of claim 15 comprising the steps of:
(a) combining an ⁸⁹Zr-chloride solution with DFO-AP-101 to obtain a reaction solution;
(b) warming the reaction solution to a temperature of about 37° C. for a period of about 45 minutes;
(c) recovering ⁸⁹Zr-DFO-AP-101 into a recovery solution from the reaction solution; and preferably
(d) adjustment of the ⁸⁹Zr-DFO-AP-101 recovery solution to a final volume by adding saline, preferably wherein the final volume is about 10 mL; and preferably
(e) filtering of the ⁸⁹Zr-DFO-AP-101 solution.

44. The method of claim 43, wherein the ⁸⁹Zr-chlorid solution of step (a) is provided by the following steps:
(a1) providing a solution of ⁸⁹Zr-oxalate;
(a2) adding a solution comprising HCl to the solution of (a1); and
(a3) collecting a solution comprising ⁸⁹Zr-chloride from the solution of (a2).

45. The method of claim 43 or 44 further comprising the step of:
(f) formulation of the filtered ⁸⁹Zr-DFO-AP-101 solution obtained from step (e) into a composition by addition of 0.9% saline and 10 mg of gentisic acid, preferably wherein the final volume of the composition is about 10 mL.
